SEQUENCE LISTING (484 sequences)

SEQ ID NO: 1 - protein, 510 aa, Artificial Sequence
  synthetic sequence
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 2 - skipped

SEQ ID NO: 3 - protein, 290 aa, Artificial Sequence
  synthetic sequence
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 313 aa, Artificial Sequence
  synthetic sequence
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVA

SEQ ID NO: 5 - skipped

SEQ ID NO: 6 - protein, 133 aa, Artificial Sequence [sequence omitted]

SEQ ID NO: 7 - protein, 369 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 356 aa, Artificial Sequence
  synthetic sequence
APTSSSTKKTQLQLEHLLLHLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 359 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 364 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 369 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNYHTQKSLSLSPG

SEQ ID NO: 13 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 374 aa, Artificial Sequence
  synthetic sequence
APTSSSTKKTQLQLEHLLLHLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYPVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 153 aa, Artificial Sequence
  synthetic sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATEIKHLQCLEEELKPLEEALRLAPSKNFHLRPRDLISDINVIVLELKGSETTFMCEYADETATIVEFLNRWITFSQSIISTLT

SEQ ID NO: 17 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHIQCLEEELKPLEEALRLAPSKNFHLRPRDLISDINVIVLELKGSETTFMCEYADETATIVEFLNRWITFSQSIISTLT

SEQ ID NO: 18 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHIRPRDLISDINVIVLELKGSETTFMCEYADETATIVEFLNRWITFSQSIISTLT

SEQ ID NO: 19 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHLRPRDLISDINVIVLELKGSETTFMCEYADETATIVEFINRWITFSQSIISTLT

SEQ ID NO: 20 - protein, 20 aa, Artificial Sequence
  synthetic sequence
MYRMQLLSCIALSLALVTNS

SEQ ID NO: 21 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 20 aa, Artificial Sequence
  synthetic sequence
GGGGSGGGGSGGGGSGGGGS

SEQ ID NO: 23 - protein, 5 aa, Artificial Sequence
  synthetic sequence
GGGGS

SEQ ID NO: 24 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATEIKHLQCLEEELKPLEEALRLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHIQCLEEELKPLEEALRLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHIRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHLRPRDLISDINVIVLELKGSETTFMCEYADETATIVEFINRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 226 aa, Artificial Sequence
  synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 5 aa, Artificial Sequence
  synthetic sequence
GGGGA

SEQ ID NO: 30 - protein, 15 aa, Artificial Sequence
  synthetic sequence
GGGGSGGGGSGGGGS

SEQ ID NO: 31 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 32 - protein, 133 aa, Artificial Sequence
  synthetic sequence
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 33 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 34 - protein, 133 aa, Artificial Sequence [sequence omitted]

SEQ ID NO: 35 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLT

SEQ ID NO: 36 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISRINVIVLELKGSETTFMCEYADETATIVEFLNRWITFSQSIISTLT

SEQ ID NO: 37 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 38 - protein, 133 aa, Artificial Sequence
  synthetic sequence
APTSSSTKKTQLQLEHLLLDLQMILNGINNYKNPKLTRMLTFKFYMPKKATEIKHLQCLEEELKPLEEALNLAPSKNFHLRPRDLISDINVIVLELKGSETTFMCEYADETATIVEFLNRWITFSQSIISTLT

SEQ ID NO: 39 - protein, 133 aa, Artificial Sequence
  synthetic sequence
APTSSSTKKTQLQLEHLLLDLQMILNGINNYKNPKLTRMLTFKFYMPKKATELKHIQCLEEELKPLEEALNLAPSKNFHLRPRDLISDINVIVLELKGSETTFMCEYADETATIVEFLNRWITFSQSIISTLT

SEQ ID NO: 40 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHIRPRDLISDINVIVLELKGSETTFMCEYADETATIVEFLNRWITFSQSIISTLT

SEQ ID NO: 41 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFINRWITFSQSIISTLT

SEQ ID NO: 42 - protein, 226 aa, Artificial Sequence; identical to SEQ ID NO: 21
  synthetic sequence

SEQ ID NO: 43 - protein, 226 aa, Artificial Sequence
  synthetic sequence
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 107 aa, Artificial Sequence [sequence omitted]

SEQ ID NO: 46 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 374 aa, Artificial Sequence
  synthetic sequence
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISRINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 379 aa, Artificial Sequence
  synthetic sequence
APTSSSTKKTQLQLEHLLLDLQMILNGINNYKNPKLTRMLTFKFYMPKKATEIKHLQCLEEELKPLEEALNLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 379 aa, Artificial Sequence
  synthetic sequence
APTSSSTKKTQLQLEHLLLDLQMILNGINNYKNPKLTRMLTFKFYMPKKATELKHIQCLEEELKPLEEALNLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHIRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFINRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 364 aa, Artificial Sequence
  synthetic sequence
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFAQSIISTLT

SEQ ID NO: 59 - protein, 379 aa, Artificial Sequence
  synthetic sequence
  MISC_FEATURE (53)..(53): At least one of X1, X2, X3, and X4 is I and the remainder are L or I.
  MISC_FEATURE (56)..(56): At least one of X1, X2, X3, and X4 is I and the remainder are L or I.
  MISC_FEATURE (80)..(80): At least one of X1, X2, X3, and X4 is I and the remainder are L or I.
  MISC_FEATURE (118)..(118): At least one of X1, X2, X3, and X4 is I and the remainder are L or I.
APTSSSTKKTQLQLEHLLLDLQMILNGINNYKNPKLTRMLTFKFYMPKKATEXKHXQCLEEELKPLEEALNLAPSKNFHXRPRDLISDINVIVLELKGSETTFMCEYADETATIVEFXNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 133 aa, Artificial Sequence
  synthetic sequence
  MISC_FEATURE (53)..(53): At least one of X1, X2, X3, and X4 is I and the remainder are L or I.
  MISC_FEATURE (56)..(56): At least one of X1, X2, X3, and X4 is I and the remainder are L or I.
  MISC_FEATURE (80)..(80): At least one of X1, X2, X3, and X4 is I and the remainder are L or I.
  MISC_FEATURE (118)..(118): At least one of X1, X2, X3, and X4 is I and the remainder are L or I.
APTSSSTKKTQLQLEHLLLDLQMILNGINNYKNPKLTRMLTFKFYMPKKATEXKHXQCLEEELKPLEEALNLAPSKNFHXRPRDLISDINVIVLELKGSETTFMCEYADETATIVEFXNRWITFSQSIISTLT

SEQ ID NO: 61-70 - skipped (10 entries)

SEQ ID NO: 71 - protein, 21 aa, Artificial Sequence
  synthetic sequence
GGGGSEGGGSEGGGSEGGGSE

SEQ ID NO: 72 - protein, 20 aa, Artificial Sequence
  synthetic sequence
GGGSEGGGSEGGGSEGGGSE

SEQ ID NO: 73 - protein, 15 aa, Artificial Sequence
  synthetic sequence
GGGSEGGGSEGGGSE

SEQ ID NO: 74 - protein, 20 aa, Artificial Sequence
  synthetic sequence
AEEEKAEEEKAEEEKAEEEK

SEQ ID NO: 75 - protein, 15 aa, Artificial Sequence
  synthetic sequence
GGGSKGGGSKGGGSK

SEQ ID NO: 76 - protein, 15 aa, Artificial Sequence
  synthetic sequence
GSAGKGSAGKGSAGK

SEQ ID NO: 77 - protein, 15 aa, Artificial Sequence
  synthetic sequence
GGGSKGGGSKGGGSK

SEQ ID NO: 78 - protein, 5 aa, Artificial Sequence
  synthetic sequence
GSAGK

SEQ ID NO: 79 - protein, 17 aa, Artificial Sequence
  synthetic sequence
GAGGGSKGGGSKGGGSK

SEQ ID NO: 80 - protein, 15 aa, Artificial Sequence
  synthetic sequence
GSAGKGSAGKGSAGK

SEQ ID NO: 81 - protein, 5 aa, Artificial Sequence
  synthetic sequence
GGGSK

SEQ ID NO: 82 - protein, 5 aa, Artificial Sequence
  synthetic sequence
AEEEK

SEQ ID NO: 83 - protein, 60 aa, Artificial Sequence
  synthetic sequence
GGSSGSGSGSTGTSSSGTGTSAGTTGTSASTSGSGSGGGGGSGGGGSAGGTATAGASSGS

SEQ ID NO: 84 - protein, 24 aa, Artificial Sequence
  synthetic sequence
MKWVTFISLLFLFSSAYSRGVFRR

SEQ ID NO: 85 - protein, 585 aa, Artificial Sequence [sequence omitted]

SEQ ID NO: 86-136 - skipped (51 entries)

SEQ ID NO: 137 - protein, 118 aa, Artificial Sequence
  synthetic sequence
QVQLVQSGAEVKKPGASVKVSCKASGGSFTGYYMHWVRQAPGQGLEWMGWINPNDGAIHYAQNFQGRVTMTRDTSTSTVYMELSSLRSEDTAVYYCARDTVTGDFDYWGQGTLVTVSS

SEQ ID NO: 138 - protein, 107 aa, Artificial Sequence
  synthetic sequence
DIQMTQSPSSLSASVGDRVTITCQASHDIDKYLNWYQQKPGKAPKLLIYAASSLQSGVPSRFSGSGSGTDFTLTISSLQPEDFATYYCQQANSLPLTFGGGTKVEIK

SEQ ID NO: 139 - protein, 9 aa, Artificial Sequence
  synthetic sequence
GSFTGYYMH

SEQ ID NO: 140 - protein, 13 aa, Artificial Sequence
  synthetic sequence
GWINPNDGAIHYA

SEQ ID NO: 141 - protein, 13 aa, Artificial Sequence
  synthetic sequence
CARDTVTGDFDYW

SEQ ID NO: 142 - protein, 11 aa, Artificial Sequence
  synthetic sequence
QASHDIDKYLN

SEQ ID NO: 143 - protein, 5 aa, Artificial Sequence
  synthetic sequence
SSLQS

SEQ ID NO: 144 - protein, 9 aa, Artificial Sequence
  synthetic sequence
QQANSLPLT

SEQ ID NO: 145 - protein, 121 aa, Artificial Sequence
  synthetic sequence
QVQLVQSGAEVKKPGASVKVSCKASGGTFSRYAVSWVRQAPGQGLEWMGWINPNSGGTSYAQRFQGRVTMTRDTSTSTVYMELSSLRSEDTAVYYCAKQGDYGGGYYFDYWGQGTLVTVSS

SEQ ID NO: 146 - protein, 107 aa, Artificial Sequence
  synthetic sequence
DIQMTQSPSSLSASVGDRVTITCRASQSISSWLAWYQQKPGKAPKLLIYKTSTLESGVPSRFSGSGSGTDFTLTISSLQPEDFATYYCQQSYSTPFTFGQGTKVEIK

SEQ ID NO: 147 - protein, 9 aa, Artificial Sequence
  synthetic sequence
GTFSRYAVS

SEQ ID NO: 148 - protein, 13 aa, Artificial Sequence
  synthetic sequence
GWINPNSGGTSYA

SEQ ID NO: 149 - protein, 16 aa, Artificial Sequence
  synthetic sequence
CAKQGDYGGGYYFDYW

SEQ ID NO: 150 - protein, 11 aa, Artificial Sequence
  synthetic sequence
RASQSISSWLA

SEQ ID NO: 151 - protein, 5 aa, Artificial Sequence
  synthetic sequence
STLES

SEQ ID NO: 152 - protein, 9 aa, Artificial Sequence
  synthetic sequence
QQSYSTPFT

SEQ ID NO: 153 - protein, 120 aa, Artificial Sequence
  synthetic sequence
QVQLVQSGAEVKKPGASVKVSCKASGGTFSSYAISWVRQAPGQGLEWMGWMNPNSGNTGYAQKFQGRVTMTRDTSTSTVYMELSSLRSEDTAVYYCARVGYSYGYGMDVWGQGTTVTVSS

SEQ ID NO: 154 - protein, 107 aa, Artificial Sequence
  synthetic sequence
DIQMTQSPSSLSASVGDRVTITCRASQSINNWLAWYQQKPGKAPKLLIYAASSLQSGVPSRFSGSGSGTDFTLTISSLQPEDFATYYCQQSYSTPFTFGPGTKVDIK

SEQ ID NO: 155 - protein, 9 aa, Artificial Sequence
  synthetic sequence
GTFSSYAIS

SEQ ID NO: 156 - protein, 13 aa, Artificial Sequence
  synthetic sequence
GWMNPNSGNTGYA

SEQ ID NO: 157 - protein, 15 aa, Artificial Sequence
  synthetic sequence
CARVGYSYGYGMDVW

SEQ ID NO: 158 - protein, 11 aa, Artificial Sequence
  synthetic sequence
RASQSINNWLA

SEQ ID NO: 159 - protein, 113 aa, Artificial Sequence
  synthetic sequence
QVQLVQSGAEVKKPGASVKVSCKASGYSFTTYYMHWVRQAPGQGLEWMGIINPSGGSTSYAQKFQGRVTMTRDTSTSTVYMELSSLRSEDTAVYYCASGWVYWGQGTLVTVSS

SEQ ID NO: 160 - protein, 107 aa, Artificial Sequence
  synthetic sequence
DIQMTQSPSSLSASVGDRVTITCQASRDIKNYLAWYQQKPGKAPKLLIYAASSLQSGVPSRFSGSGSGTDFTLTISSLQPEDFATYYCQQSYSTPPTFGPGTKVDIK

SEQ ID NO: 161 - protein, 9 aa, Artificial Sequence
  synthetic sequence
YSFTTYYMH

SEQ ID NO: 162 - protein, 13 aa, Artificial Sequence
  synthetic sequence
GIINPSGGSTSYA

SEQ ID NO: 163 - protein, 8 aa, Artificial Sequence
  synthetic sequence
CASGWVYW

SEQ ID NO: 164 - protein, 11 aa, Artificial Sequence
  synthetic sequence
QASRDIKNYLA

SEQ ID NO: 165 - protein, 9 aa, Artificial Sequence
  synthetic sequence
QQSYSTPPT

SEQ ID NO: 166 - protein, 119 aa, Artificial Sequence
  synthetic sequence
EVQLLESGGGLVQPGGSLRLSCAASGFTFSSYAMSWVRQAPGKGLEWVAAIWSDGSHQYYADSVKGRFTISRDNSKNTLYLQMNSLRAEDTAVYYCARGLGVERGLDYWGQGTLVTVSS

SEQ ID NO: 167 - protein, 107 aa, Artificial Sequence
  synthetic sequence
DIQMTQSPSSLSASVGDRVTITCRASQSISSWLAWYQQKPGKAPKLLIYAASTLQSGVPSRFSGSGSGTDFTLTISSLQPEDFATYYCQQSYSTPLTFGQGTKVEIK

SEQ ID NO: 168 - protein, 9 aa, Artificial Sequence
  synthetic sequence
FTFSSYAMS

SEQ ID NO: 169 - protein, 13 aa, Artificial Sequence
  synthetic sequence
AAIWSDGSHQYYA

SEQ ID NO: 170 - protein, 14 aa, Artificial Sequence
  synthetic sequence
CARGLGVERGLDYW

SEQ ID NO: 171 - protein, 5 aa, Artificial Sequence
  synthetic sequence
STLQS

SEQ ID NO: 172 - protein, 9 aa, Artificial Sequence
  synthetic sequence
QQSYSTPLT

SEQ ID NO: 173 - protein, 124 aa, Artificial Sequence
  synthetic sequence
EVQLLESGGGLVQPGGSLRLSCAASGFTFSNYPMHWVRQAPGKGLEWVALISDDGTNEHYADSVKGRFTISRDNSKNTLYLQMNSLRAEDTAVYYCARDSKFANYYYYYDMDVWGQGTTVTVSS

SEQ ID NO: 174 - protein, 107 aa, Artificial Sequence
  synthetic sequence
DIQMTQSPSSLSASVGDRVTITCRASQSINNYLSWYQQKPGKAPKLLIYDASNLETGVPSRFSGSGSGTDFTLTISSLQPEDFATYYCQQSYSTPLTFGPGTKVDIK

SEQ ID NO: 175 - protein, 9 aa, Artificial Sequence
  synthetic sequence
FTFSNYPMH

SEQ ID NO: 176 - protein, 13 aa, Artificial Sequence
  synthetic sequence
ALISDDGTNEHYA

SEQ ID NO: 177 - protein, 19 aa, Artificial Sequence
  synthetic sequence
CARDSKFANYYYYYDMDVW

SEQ ID NO: 178 - protein, 11 aa, Artificial Sequence
  synthetic sequence
RASQSINNYLS

SEQ ID NO: 179 - protein, 5 aa, Artificial Sequence
  synthetic sequence
SNLET

SEQ ID NO: 180 - protein, 124 aa, Artificial Sequence
  synthetic sequence
QVQLVQSGAEVKKPGASVKVSCKASGYSFTGHYIHWVRQAPGQGLEWMGIINPNGGSTTYAQKLQGRVTMTRDTSTSTVYMELSSLRSEDTAVYYCARGKFDFYGDYVTAFDIWGQGTMVTVSS

SEQ ID NO: 181 - protein, 113 aa, Artificial Sequence
  synthetic sequence
DIVMTQSPDSLAVSLGERATINCRSSQSILYSSNNRDYLAWYQQKPGQPPKLLIYWASTRQSGVPDRFSGSGSGTDFTLTISSLQAEDVAVYYCQQYYSIPVTFGGGTKVEIK

SEQ ID NO: 182 - protein, 9 aa, Artificial Sequence
  synthetic sequence
YSFTGHYIH

SEQ ID NO: 183 - protein, 13 aa, Artificial Sequence
  synthetic sequence
GIINPNGGSTTYA

SEQ ID NO: 184 - protein, 19 aa, Artificial Sequence
  synthetic sequence
CARGKFDFYGDYVTAFDIW

SEQ ID NO: 185 - protein, 17 aa, Artificial Sequence
  synthetic sequence
RSSQSILYSSNNRDYLA

SEQ ID NO: 186 - protein, 5 aa, Artificial Sequence
  synthetic sequence
STRQS

SEQ ID NO: 187 - protein, 9 aa, Artificial Sequence
  synthetic sequence
QQYYSIPVT

SEQ ID NO: 188 - protein, 128 aa, Artificial Sequence
  synthetic sequence
QVQLVQSGAEVKKPGASVKVSCKASGYTFSNYDMNWVRQAPGQGLEWMGWMNPNSGHTGSAPKFQGRVTMTRDTSTSTVYMELSSLRSEDTAVYYCARGAFGLHLGELSLHYYGMDVWGQGTTVTVSS

SEQ ID NO: 189 - protein, 107 aa, Artificial Sequence
  synthetic sequence
DIQMTQSPSSLSASVGDRVTITCRASQSINNWLAWYQQKPGKAPKLLIYAASSLQGGVPSRFSGSGSGTDFTLTISSLQPEDFATYYCQQSYSFPYTFGQGTKLEIK

SEQ ID NO: 190 - protein, 9 aa, Artificial Sequence
  synthetic sequence
YTFSNYDMN

SEQ ID NO: 191 - protein, 13 aa, Artificial Sequence
  synthetic sequence
GWMNPNSGHTGSA

SEQ ID NO: 192 - protein, 23 aa, Artificial Sequence
  synthetic sequence
CARGAFGLHLGELSLHYYGMDVW

SEQ ID NO: 193 - protein, 5 aa, Artificial Sequence
  synthetic sequence
SSLQG

SEQ ID NO: 194 - protein, 9 aa, Artificial Sequence
  synthetic sequence
QQSYSFPYT

SEQ ID NO: 195 - protein, 117 aa, Artificial Sequence
  synthetic sequence
QVQLVQSGAEVKKPGSSVKVSCKASGYTFTGYYMHWVRQAPGQGLEWMGKIVPMFDAANYAPKFQGRVTITADESTSTAYMELSSLRSEDTAVYYCARGPKWELDTWGQGTLVTVSS

SEQ ID NO: 196 - protein, 107 aa, Artificial Sequence
  synthetic sequence
DIQMTQSPSSLSASVGDRVTITCRASQSISRWLAWYQQKPGKAPKLLIYGASSLQSGVPSRFSGSGSGTDFTLTISSLQPEDFATYYCQQANSFPVTFGGGTKVEIK

SEQ ID NO: 197 - protein, 9 aa, Artificial Sequence
  synthetic sequence
YTFTGYYMH

SEQ ID NO: 198 - protein, 13 aa, Artificial Sequence
  synthetic sequence
GKIVPMFDAANYA

SEQ ID NO: 199 - protein, 12 aa, Artificial Sequence
  synthetic sequence
CARGPKWELDTW

SEQ ID NO: 200 - protein, 11 aa, Artificial Sequence
  synthetic sequence
RASQSISRWLA

SEQ ID NO: 201 - protein, 9 aa, Artificial Sequence
  synthetic sequence
QQANSFPVT

SEQ ID NO: 202 - protein, 124 aa, Artificial Sequence
  synthetic sequence
QVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCAKTAGYDWLPSGLGMDVWGQGTTVTVSS

SEQ ID NO: 203 - protein, 107 aa, Artificial Sequence
  synthetic sequence
DIQMTQSPSSLSASVGDRVTITCRASQSINSWLAWYQQKPGKAPKLLIYYASSLQSGVPSRFSGSGSGTDFTLTISSLQPEDFATYYCQQGYSVPLSFGQGTKLEIK

SEQ ID NO: 204 - protein, 19 aa, Artificial Sequence
  synthetic sequence
CAKTAGYDWLPSGLGMDVW

SEQ ID NO: 205 - protein, 11 aa, Artificial Sequence
  synthetic sequence
RASQSINSWLA

SEQ ID NO: 206 - protein, 9 aa, Artificial Sequence
  synthetic sequence
QQGYSVPLS

SEQ ID NO: 207 - protein, 118 aa, Artificial Sequence
  synthetic sequence
QVQLVQSGAEVKKPGSSVKVSCKASGYTFSNYGITWVRQAPGQGLEWMGGIIPIFGSTASYAQKFQGRVTITADESTSTAYMELSSLRSEDTAVYYCARWRSDAFDIWGQGTMVTVSS

SEQ ID NO: 208 - protein, 107 aa, Artificial Sequence
  synthetic sequence
DIQMTQSPSSLSASVGDRVTITCRASQGISNWLAWYQQKPGKAPKLLIYDASNLETGVPSRFSGSGSGTDFTLTISSLQPEDFATYYCQQSYSTPLTFGGGTKVEIK

SEQ ID NO: 209 - protein, 9 aa, Artificial Sequence
  synthetic sequence
YTFSNYGIT

SEQ ID NO: 210 - protein, 14 aa, Artificial Sequence
  synthetic sequence
GGIIPIFGSTASYA

SEQ ID NO: 211 - protein, 12 aa, Artificial Sequence
  synthetic sequence
CARWRSDAFDIW

SEQ ID NO: 212 - protein, 11 aa, Artificial Sequence
  synthetic sequence
RASQGISNWLA

SEQ ID NO: 213 - protein, 120 aa, Artificial Sequence
  synthetic sequence
QVQLVQSGAEVKKPGASVKVSCKASGGTFSTYAISWVRQAPGQGLEWMGWINPNSGGTNYAQKFQGRVTMTRDTSTSIVYMELSSLRSEDTAVYYCARVNYDFYYGMDVWGQGTTVTVSS

SEQ ID NO: 214 - protein, 107 aa, Artificial Sequence
  synthetic sequence
DIQMTQSPSSLSASVGDRVTITCRASQGIRNDLGWYQQKPGKAPKLLIYRASTLNSGVPSRFSGSGSGTDFTLTISSLQPEDFATYYCQQSYSTPFTFGPGTKVDIK

SEQ ID NO: 215 - protein, 9 aa, Artificial Sequence
  synthetic sequence
GTFSTYAIS

SEQ ID NO: 216 - protein, 13 aa, Artificial Sequence
  synthetic sequence
GWINPNSGGTNYA

SEQ ID NO: 217 - protein, 15 aa, Artificial Sequence
  synthetic sequence
CARVNYDFYYGMDVW

SEQ ID NO: 218 - protein, 11 aa, Artificial Sequence
  synthetic sequence
RASQGIRNDLG

SEQ ID NO: 219 - protein, 5 aa, Artificial Sequence
  synthetic sequence
STLNS

SEQ ID NO: 220 - protein, 120 aa, Artificial Sequence
  synthetic sequence
QVQLVQSGAEVKKPGASVKVSCKASGGTFSTYAISWVRQAPGQGLEWMGWINPNSGGTNYAQKFQGRVTMTRDTSTSTVYMELSSLRSEDTAVYYCARVNYDFYYGMDVWGQGTTVTVSS

SEQ ID NO: 221 - protein, 118 aa, Artificial Sequence
  synthetic sequence
EVQLLESGGGLVQPGGSLRLSCAASGFSFSSYDMSWVRQAPGKGLEWVSGISGSGSSTYYADSVKGRFTISRDNSKNTLYLQMNSLRAEDTAVYYCASPYGMGYMDVWGKGTTVTVSS

SEQ ID NO: 222 - protein, 107 aa, Artificial Sequence
  synthetic sequence
DIQMTQSPSSLSASVGDRVTITCRASQDIANYLAWYQQKPGKAPKLLIYGASSVQTGVPSRFSGSGSGTDFTLTISSLQPEDFATYYCQQSYTTPYTFGQGTRLEIK

SEQ ID NO: 223 - protein, 9 aa, Artificial Sequence
  synthetic sequence
FSFSSYDMS

SEQ ID NO: 224 - protein, 13 aa, Artificial Sequence
  synthetic sequence
SGISGSGSSTYYA

SEQ ID NO: 225 - protein, 13 aa, Artificial Sequence
  synthetic sequence
CASPYGMGYMDVW

SEQ ID NO: 226 - protein, 11 aa, Artificial Sequence
  synthetic sequence
RASQDIANYLA

SEQ ID NO: 227 - protein, 5 aa, Artificial Sequence
  synthetic sequence
SSVQT

SEQ ID NO: 228 - protein, 9 aa, Artificial Sequence
  synthetic sequence
QQSYTTPYT

SEQ ID NO: 229 - protein, 120 aa, Artificial Sequence
  synthetic sequence
QVQLVQSGAEVKKPGASVKVSCKASGGSFNNYAISWVRQAPGQGLEWMGWINPNTGGTSYAQKFQGRVTMTRDTSTSTVYMELSSLRSEDTAVYYCARVSYGVGYYMDVWGKGTTVTVSS

SEQ ID NO: 230 - protein, 107 aa, Artificial Sequence
  synthetic sequence
DIQMTQSPSSLSASVGDRVTITCQASQDISRYLNWYQQKPGKAPKLLIYAASNLQSGVPSRFSGSGSGTDFTLTISSLQPEDFATYYCQQSYSTPFTFGPGTKVDIK

SEQ ID NO: 231 - protein, 9 aa, Artificial Sequence
  synthetic sequence
GSFNNYAIS

SEQ ID NO: 232 - protein, 13 aa, Artificial Sequence
  synthetic sequence
GWINPNTGGTSYA

SEQ ID NO: 233 - protein, 15 aa, Artificial Sequence
  synthetic sequence
CARVSYGVGYYMDVW

SEQ ID NO: 234 - protein, 11 aa, Artificial Sequence
  synthetic sequence
QASQDISRYLN

SEQ ID NO: 235 - protein, 5 aa, Artificial Sequence
  synthetic sequence
SNLQS

SEQ ID NO: 236 - protein, 107 aa, Artificial Sequence
  synthetic sequence
DIQMTQSPSSLSASVGDRVTITCQASQDISRYLNWYQQKPGKAPKLLIYAASNLQSGVPSRFSGSGSGTDSTLTISSLQPEDFATYYCQQSYSTPFTFGPGTKVDIK

SEQ ID NO: 237 - protein, 129 aa, Artificial Sequence
  synthetic sequence
QVQLVQSGAEVKKPGASVKVSCKASGYTFTDDYIHWVRQAPGQGLEWMGWMNTNSGNTGYAQKFQGRVTMTRDTSTSTVYMELSSLRSEDTAVYYCARGGSYSSGWYGRLDYYYGMDVWGQGTTVTVSS

SEQ ID NO: 238 - protein, 107 aa, Artificial Sequence
  synthetic sequence
DIQMTQSPSSLSASVGDRVTITCRASQGVGNALGWYQQKPGKAPKLLIYAASSLQSGVPSRFSGSGSGTDFTLTISSLQPEDFATYYCQQAYSFPWTFGQGTKLEIK

SEQ ID NO: 239 - protein, 9 aa, Artificial Sequence
  synthetic sequence
YTFTDDYIH

SEQ ID NO: 240 - protein, 13 aa, Artificial Sequence
  synthetic sequence
GWMNTNSGNTGYA

SEQ ID NO: 241 - protein, 24 aa, Artificial Sequence
  synthetic sequence
CARGGSYSSGWYGRLDYYYGMDVW

SEQ ID NO: 242 - protein, 11 aa, Artificial Sequence
  synthetic sequence
RASQGVGNALG

SEQ ID NO: 243 - protein, 9 aa, Artificial Sequence
  synthetic sequence
QQAYSFPWT

SEQ ID NO: 244 - protein, 113 aa, Artificial Sequence
  synthetic sequence
QVQLVQSGAEVKKPGASVKVSCKASGYTFTDYAMHWVRQAPGQGLEWMGWLNPNSGNTGYAPKFQGRVTMTRDTSTSTVYMELSSLRSEDTAVYYCAAGLFIWGQGTMVTVSS

SEQ ID NO: 245 - protein, 107 aa, Artificial Sequence
  synthetic sequence
DIQMTQSPSSLSASVGDRVTITCRASQSINRWLAWYQQKPGKAPKLLIYDASSLESGVPSRFSGSGSGTDFTLTISSLQPEDFATYYCQQSYSIPITFGQGTRLEIK

SEQ ID NO: 246 - protein, 9 aa, Artificial Sequence
  synthetic sequence
YTFTDYAMH

SEQ ID NO: 247 - protein, 13 aa, Artificial Sequence
  synthetic sequence
GWLNPNSGNTGYA

SEQ ID NO: 248 - protein, 8 aa, Artificial Sequence
  synthetic sequence
CAAGLFIW

SEQ ID NO: 249 - protein, 11 aa, Artificial Sequence
  synthetic sequence
RASQSINRWLA

SEQ ID NO: 250 - protein, 5 aa, Artificial Sequence
  synthetic sequence
SSLES

SEQ ID NO: 251 - protein, 9 aa, Artificial Sequence
  synthetic sequence
QQSYSIPIT

SEQ ID NO: 252 - protein, 120 aa, Artificial Sequence
  synthetic sequence
QVQLVQSGAEVKKPGSSVKVSCKASGGTFSSYAISWVRQAPGQGLEWMGGIIPGFGSPNYAPNFQGRVTITADESTSTAYMELSSLRSEDTAVYYCTTEYCSSTSCSDYWGQGTLVTVSS

SEQ ID NO: 253 - protein, 112 aa, Artificial Sequence
  synthetic sequence
DIVMTQSPLSLPVTPGEPASISCRSSQSLLHSNGYNYLDWYLQKPGQSPQLLIYQGSNRAPGVPDRFSGSGSGTDFTLKISRVEAEDVGVYYCMQALQTPLTFGQGTKVEIK

SEQ ID NO: 254 - protein, 13 aa, Artificial Sequence
  synthetic sequence
GGIIPGFGSPNYA

SEQ ID NO: 255 - protein, 15 aa, Artificial Sequence
  synthetic sequence
CTTEYCSSTSCSDYW

SEQ ID NO: 256 - protein, 16 aa, Artificial Sequence
  synthetic sequence
RSSQSLLHSNGYNYLD

SEQ ID NO: 257 - protein, 5 aa, Artificial Sequence
  synthetic sequence
SNRAP

SEQ ID NO: 258 - protein, 9 aa, Artificial Sequence
  synthetic sequence
MQALQTPLT

SEQ ID NO: 259 - protein, 125 aa, Artificial Sequence
  synthetic sequence
QVQLVQSGAEVKKPGASVKVSCKASGYTFSDHYMHWVRQAPGQGLEWMGTINPSGGRTSYAQKFQGRVTMTRDTSTSTVYMELSSLRSEDTAVYYCAADNGHASGWLYYYGMDVWGQGTTVTVSS

SEQ ID NO: 260 - protein, 107 aa, Artificial Sequence
  synthetic sequence
DIQMTQSPSSLSASVGDRVTITCRASQSISNWVAWYQQKPGKAPKLLIYRASTLQSGVPSRFSGSGSGTDFTLTISSLQPEDFATYYCQQSHSLPLTFGPGTKVDIK

SEQ ID NO: 261 - protein, 9 aa, Artificial Sequence
  synthetic sequence
YTFSDHYMH

SEQ ID NO: 262 - protein, 13 aa, Artificial Sequence
  synthetic sequence
GTINPSGGRTSYA

SEQ ID NO: 263 - protein, 20 aa, Artificial Sequence
  synthetic sequence
CAADNGHASGWLYYYGMDVW

SEQ ID NO: 264 - protein, 11 aa, Artificial Sequence
  synthetic sequence
RASQSISNWVA

SEQ ID NO: 265 - protein, 9 aa, Artificial Sequence
  synthetic sequence
QQSHSLPLT

SEQ ID NO: 266 - protein, 116 aa, Artificial Sequence
  synthetic sequence
EVQLLESGGGLVQPGGSLRLSCAASGFTFSSYAMSWVRQAPGKGLEWVSGISGGGGTTYYADSVKGRFTISRDNSKNTLYLQMNSLRAEDTAVYYCASEYYGMDVWGQGTTVTVSS

SEQ ID NO: 267 - protein, 107 aa, Artificial Sequence
  synthetic sequence
DIQMTQSPSSLSASVGDRVTITCRASQSISGWLAWYQQKPGKAPKLLIYAASSLQSGVPSRFSGSGSGTDFTLTISSLQPEDFATYYCQQYRNFPYTFGQGTKLEIK

SEQ ID NO: 268 - protein, 13 aa, Artificial Sequence
  synthetic sequence
SGISGGGGTTYYA

SEQ ID NO: 269 - protein, 11 aa, Artificial Sequence
  synthetic sequence
CASEYYGMDVW

SEQ ID NO: 270 - protein, 11 aa, Artificial Sequence
  synthetic sequence
RASQSISGWLA

SEQ ID NO: 271 - protein, 9 aa, Artificial Sequence
  synthetic sequence
QQYRNFPYT

SEQ ID NO: 272 - protein, 113 aa, Artificial Sequence
  synthetic sequence
QVQLVQSGAEVKKPGASVKVSCKASGYTFSGYYMHWVRQAPGQGLEWMGVINPSGGSTSYAQKFQGRVTMTRDTSTSTVYMELSSLRSEDTAVYYCAEGFDYWGQGTLVTVSS

SEQ ID NO: 273 - protein, 107 aa, Artificial Sequence
  synthetic sequence
EIVMTQSPATLSVSPGERATLSCRASQGVGRSLAWYQQKPGQAPRLLIYGASTRATGIPARFSGSGSGTEFTLTISSLQSEDFAVYYCQQYYTTPITFGQGTRLEIK

SEQ ID NO: 274 - protein, 9 aa, Artificial Sequence
  synthetic sequence
YTFSGYYMH

SEQ ID NO: 275 - protein, 13 aa, Artificial Sequence
  synthetic sequence
GVINPSGGSTSYA

SEQ ID NO: 276 - protein, 8 aa, Artificial Sequence
  synthetic sequence
CAEGFDYW

SEQ ID NO: 277 - protein, 11 aa, Artificial Sequence
  synthetic sequence
RASQGVGRSLA

SEQ ID NO: 278 - protein, 5 aa, Artificial Sequence
  synthetic sequence
STRAT

SEQ ID NO: 279 - protein, 9 aa, Artificial Sequence
  synthetic sequence
QQYYTTPIT

SEQ ID NO: 280 - protein, 120 aa, Artificial Sequence
  synthetic sequence
QVQLVQSGAEVKKPGASVKVSCKASGGTFSNYAISWVRQAPGQGLEWMGWMNPNSGNTGYAQKFQGRVTMTRDTSTSTVYMELSSLRSEDTAVYYCARVNYYYYYGMDVWGQGTTVTVSS

SEQ ID NO: 281 - protein, 107 aa, Artificial Sequence
  synthetic sequence
DIQMTQSPSSLSASVGDRVTITCQASQDISNYLNWYQQKPGKAPKLLIYKASTLKSGVPSRFSGSGSGTDFTLTISSLQPEDFATYYCQQADNLPFTFGPGTKVDIK

SEQ ID NO: 282 - protein, 9 aa, Artificial Sequence
  synthetic sequence
GTFSNYAIS

SEQ ID NO: 283 - protein, 15 aa, Artificial Sequence
  synthetic sequence
CARVNYYYYYGMDVW

SEQ ID NO: 284 - protein, 11 aa, Artificial Sequence
  synthetic sequence
QASQDISNYLN

SEQ ID NO: 285 - protein, 5 aa, Artificial Sequence
  synthetic sequence
STLKS

SEQ ID NO: 286 - protein, 9 aa, Artificial Sequence
  synthetic sequence
QQADNLPFT

SEQ ID NO: 287 - protein, 122 aa, Artificial Sequence
  synthetic sequence
QVQLVQSGAEVKKPGASVKVSCKASGYTFTNYYMHWVRQAPGQGLEWMGIINPSGGSTSYAQRFQGRVTMTRDTSTSTVYMELSSLRSEDTAVYYCARDWGWDYYYYGMDVWGQGTTVTVSS

SEQ ID NO: 288 - protein, 107 aa, Artificial Sequence
  synthetic sequence
DIQMTQSPSSLSASVGDRVTITCQASRDISNYLNWYQQKPGKAPKLLIYAASSLQSGVPSRFSGSGSGTDFTLTISSLQPEDFATYYCQQANSFPPTFGQGTKLEIK

SEQ ID NO: 289 - protein, 9 aa, Artificial Sequence
  synthetic sequence
YTFTNYYMH

SEQ ID NO: 290 - protein, 17 aa, Artificial Sequence
  synthetic sequence
CARDWGWDYYYYGMDVW

SEQ ID NO: 291 - protein, 11 aa, Artificial Sequence
  synthetic sequence
QASRDISNYLN

SEQ ID NO: 292 - protein, 9 aa, Artificial Sequence
  synthetic sequence
QQANSFPPT

SEQ ID NO: 293 - protein, 119 aa, Artificial Sequence
  synthetic sequence
EVQLLESGGGLVQPGGSLRLSCAASGFTFSNSDMSWVRQAPGKGLEWVSGITISGGSTYYADSVRGRFTISRDNSKNTLYLQMSSLRAEDTAVYYCARGRGGSGWLDYWGQGTLVTVSS

SEQ ID NO: 294 - protein, 113 aa, Artificial Sequence
  synthetic sequence
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYTTPPTFGQGTRLEIK

SEQ ID NO: 295 - protein, 9 aa, Artificial Sequence
  synthetic sequence
FTFSNSDMS

SEQ ID NO: 296 - protein, 13 aa, Artificial Sequence
  synthetic sequence
SGITISGGSTYYA

SEQ ID NO: 297 - protein, 14 aa, Artificial Sequence
  synthetic sequence
CARGRGGSGWLDYW

SEQ ID NO: 298 - protein, 17 aa, Artificial Sequence
  synthetic sequence
KSSQSVLYSPNNKNYLA

SEQ ID NO: 299 - protein, 5 aa, Artificial Sequence
  synthetic sequence
STRES

SEQ ID NO: 300 - protein, 9 aa, Artificial Sequence
  synthetic sequence
QQYYTTPPT

SEQ ID NO: 301 - protein, 107 aa, Artificial Sequence
  synthetic sequence
DIQMTQSPSSLSASVGDRVTITCRASQSISRWLAWYQQKPGKAPKLLIYGASSLQSGVPSRFSGSGSGTDFTLTISSLQPEDFATYYCQQANSFPVTFGGGTKVDIK

SEQ ID NO: 302 - protein, 107 aa, Artificial Sequence
  synthetic sequence
DIQMTQSPSSLSASVGDRVTITCRASQSISRWLAWYQQKPGKAPKLLIYGASSLQSGVPSRFSGSGSGTDFTFTISSLQPEDFATYYCQQANSFPVTFGGGTKVDIK

SEQ ID NO: 303 - protein, 120 aa, Artificial Sequence
  synthetic sequence
QVQLVQSGAEVKKPGSSVKVSCKASGGDFSNYFVSWVRQAPGQGLEWMGWINPHNGDTMYAQKFQGRVTITADESTSTAYMELSSLRSEDTAVYYCARGGYSYGYTFDIWGQGTMVTVSS

SEQ ID NO: 304 - protein, 107 aa, Artificial Sequence
  synthetic sequence
DIQMTQSPSSLSASVGDRVTITCRASQSISTWLAWYQQKPGKAPKLLIYAASTLQSGVPSRFSGSGSGTDFTLTISSLQPEDFATYYCQQSYSTPFTFGGGTKVEIK

SEQ ID NO: 305 - protein, 9 aa, Artificial Sequence
  synthetic sequence
GDFSNYFVS

SEQ ID NO: 306 - protein, 13 aa, Artificial Sequence
  synthetic sequence
GWINPHNGDTMYA

SEQ ID NO: 307 - protein, 15 aa, Artificial Sequence
  synthetic sequence
CARGGYSYGYTFDIW

SEQ ID NO: 308 - protein, 11 aa, Artificial Sequence
  synthetic sequence
RASQSISTWLA

SEQ ID NO: 309 - protein, 119 aa, Artificial Sequence
  synthetic sequence
EVQLLESGGGLVQPGGSLRLSCAASGFTFSNSDMSWVRQAPGKGLEWVSGITISGGSTYYADSVKGRFTISRDNSKNTLYLQMNSLRAEDTAVYYCARGRGGSGWLDYWGQGTLVTVSS

SEQ ID NO: 310 - protein, 113 aa, Artificial Sequence
  synthetic sequence
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYITPPTFGQGTRLEIK

SEQ ID NO: 311 - protein, 9 aa, Artificial Sequence
  synthetic sequence
QQYYITPPT

SEQ ID NO: 312 - protein, 113 aa, Artificial Sequence
  synthetic sequence
QVQLVQSGAEVKKPGSSVKVSCKASGHTFTDYYMHWVRQAPGQGLEWMGIINPSGGSTSYAQKFQGRVTITADESTSTAYMELSSLRSEDTAVYYCASGWTDWGQGTLVTVSS

SEQ ID NO: 313 - protein, 108 aa, Artificial Sequence
  synthetic sequence
EIVMTQSPATLSVSPGERATLSCRASQSVSSYLAWYQQKPGQAPRLLIYGTSSRATGIPARFSGSGSGTEFTLTISSLQSEDFAVYYCQQYTTSPITFGQGTRLEIKR

SEQ ID NO: 314 - protein, 9 aa, Artificial Sequence
  synthetic sequence
HTFTDYYMH

SEQ ID NO: 315 - protein, 8 aa, Artificial Sequence
  synthetic sequence
CASGWTDW

SEQ ID NO: 316 - protein, 11 aa, Artificial Sequence
  synthetic sequence
RASQSVSSYLA

SEQ ID NO: 317 - protein, 5 aa, Artificial Sequence
  synthetic sequence
SSRAT

SEQ ID NO: 318 - protein, 9 aa, Artificial Sequence
  synthetic sequence
QQYTTSPIT

SEQ ID NO: 319 - protein, 117 aa, Artificial Sequence
  synthetic sequence
QVQLVQSGAEVKKPGASVKVSCKASGYTFTDYYMHWVRQAPGQGLEWMGGIFPVFGSSTYAQKFQGRVTMTRDTSTSTVYMELSSLRSEDTAVYYCARDHGSGLDVWGQGTTVTVSS

SEQ ID NO: 320 - protein, 107 aa, Artificial Sequence
  synthetic sequence
DIQMTQSPSSLSASVGDRVTITCQASQDISNYLNWYQQKPGKAPKLLIYDAKDLHPGVPSRFSGSGSGTDFTLTISSLQPEDFATYYCQESFSTLTFGQGTKVEIKR

SEQ ID NO: 321 - protein, 9 aa, Artificial Sequence
  synthetic sequence
YTFTDYYMH

SEQ ID NO: 322 - protein, 13 aa, Artificial Sequence
  synthetic sequence
GGIFPVFGSSTYA

SEQ ID NO: 323 - protein, 12 aa, Artificial Sequence
  synthetic sequence
CARDHGSGLDVW

SEQ ID NO: 324 - protein, 5 aa, Artificial Sequence
  synthetic sequence
KDLHP

SEQ ID NO: 325 - protein, 8 aa, Artificial Sequence
  synthetic sequence
QESFSTLT

SEQ ID NO: 326 - protein, 113 aa, Artificial Sequence
  synthetic sequence
QVQLVQSGAEVKKPGASVKVSCKASGYSFTTYYMHWVRQAPGQGLEWMGIIAPSGGSTSYAQKFQGRVTMTRDTSTSTVYMELSSLRSEDTAVYYCASGWVYWGQGTLVTVSS

SEQ ID NO: 327 - protein, 13 aa, Artificial Sequence
  synthetic sequence
GIIAPSGGSTSYA

SEQ ID NO: 328 - protein, 113 aa, Artificial Sequence
  synthetic sequence
QVQLVQSGAEVKKPGASVKVSCKASGYSFTTYYMHWVRQAPGQGLEWMGIIGPSGGSTSYAQKFQGRVTMTRDTSTSTVYMELSSLRSEDTAVYYCASGWVYWGQGTLVTVSS

SEQ ID NO: 329 - protein, 125 aa, Artificial Sequence
  synthetic sequence
QVQLVQSGAEVKKPGASVKVSCKASGYTFSDHYMHWVRQAPGQGLEWMGTIAPSGGRTSYAQKFQGRVTMTRDTSTSTVYMELSSLRSEDTAVYYCAADNGHASGWLYYYGMDVWGQGTTVTVSS

SEQ ID NO: 330 - protein, 13 aa, Artificial Sequence
  synthetic sequence
GTIAPSGGRTSYA

SEQ ID NO: 331 - protein, 125 aa, Artificial Sequence
  synthetic sequence
QVQLVQSGAEVKKPGASVKVSCKASGYTFSDHYMHWVRQAPGQGLEWMGTIGPSGGRTSYAQKFQGRVTMTRDTSTSTVYMELSSLRSEDTAVYYCAADNGHASGWLYYYGMDVWGQGTTVTVSS

SEQ ID NO: 332 - protein, 5 aa, Artificial Sequence
  synthetic sequence
TYYMH

SEQ ID NO: 333 - protein, 17 aa, Artificial Sequence
  synthetic sequence
IINPSGGSTSYAQKFQG

SEQ ID NO: 334 - protein, 6 aa, Artificial Sequence
  synthetic sequence
ASGWVY

SEQ ID NO: 335 - protein, 7 aa, Artificial Sequence
  synthetic sequence
AASSLQS

SEQ ID NO: 336 - protein, 6 aa, Artificial Sequence
  synthetic sequence
SNSDMS

SEQ ID NO: 337 - protein, 16 aa, Artificial Sequence
  synthetic sequence
GITISGGSTYYADSVK

SEQ ID NO: 338 - protein, 12 aa, Artificial Sequence
  synthetic sequence
ARGRGGSGWLDY

SEQ ID NO: 339 - protein, 7 aa, Artificial Sequence
  synthetic sequence
WASTRES

SEQ ID NO: 340-444 - skipped (105 entries)

SEQ ID NO: 445 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 446 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRSEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 447 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTEPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 448 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQTEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 449 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCRSGTYILSLLLQGYHFTADSWEHIHFIGKIQGSDAGWTLGYMLNLTNMIPAEQPLSTPLSHSTYV

SEQ ID NO: 450 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYISSLLLQGYHFTADSWEHIHFIGKIQGSDAGWTLGYMLNLTNMIPAEQPLSTPLSHSTYV

SEQ ID NO: 451 - protein, 643 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSNVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLSQGYHFTADSWEHIHFIGKIQGSDAGWTLGYMLNLTNMIPAEQPLSTPLSHSTYV

SEQ ID NO: 452 - protein, 658 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 453 - protein, 658 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVGGGSEGGGSEGGGSEGGGSEDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 454 - protein, 658 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVAEEEKAEEEKAEEEKAEEEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 455 - protein, 658 aa, Artificial Sequence; identical to SEQ ID NO: 452
  synthetic sequence

SEQ ID NO: 456 - protein, 658 aa, Artificial Sequence; identical to SEQ ID NO: 453
  synthetic sequence

SEQ ID NO: 457 - protein, 658 aa, Artificial Sequence; identical to SEQ ID NO: 454
  synthetic sequence

SEQ ID NO: 458 - protein, 658 aa, Artificial Sequence; identical to SEQ ID NO: 452
  synthetic sequence

SEQ ID NO: 459 - protein, 658 aa, Artificial Sequence; identical to SEQ ID NO: 453
  synthetic sequence

SEQ ID NO: 460 - protein, 658 aa, Artificial Sequence; identical to SEQ ID NO: 454
  synthetic sequence

SEQ ID NO: 461 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 462 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 463 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 464 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 465 - protein, 21 aa, Artificial Sequence
  synthetic sequence
EPKSCCVECPPCPAPPAAAGA

SEQ ID NO: 466 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 467 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 468 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 469 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 470 - protein, 441 aa, Artificial Sequence
  synthetic sequence
TQNKPLPENVKYGIVLDAGSSHTNLYIYKWPAEKENDTGVVQQLEECQVKGPGISKYAQKTDEIGAYLAECMELSTELIPTSKHHQTPVYLGATAGMRLLRMESEQSADEVLAAVSTSLKGYPFDFQGAKIITGQEEGAYGWITINYLLGRFTQEQSWLSLISDSQKQETFGALDLGGASTQITFVPQNSTIESPENSLQFRLYGEDYTVYTHSFLCYGKDQALWQKLAKDIQVSSGGVLKDPCFNPGYEKVVNVSELYGTPCTERFEKKLPFDQFRIQGTGDYEQCHQSILELFNNSHCPYSQCAFNGVFLPPLHGSFGAFSAFYFVMDFFKKVAKNSVISQEKMTEITKNFCSKSWEETKTSYPSVKEKYLSEYCFSGAYILSLLQGYNFTDSSWEQIHFMGKIKDSNAGWTLGYMLNLTNMIPAEQPLSPPLPHSTYI

SEQ ID NO: 471 - protein, 441 aa, Artificial Sequence
  synthetic sequence
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 472 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 473 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEAGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 474 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRSEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 475 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTEPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 476 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQTEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 477 - skipped

SEQ ID NO: 478 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCRSGTYILSLLLQGYHFTADSWEHIHFIGKIQGSDAGWTLGYMLNLTNMIPAEQPLSTPLSHSTYV

SEQ ID NO: 479 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYISSLLLQGYHFTADSWEHIHFIGKIQGSDAGWTLGYMLNLTNMIPAEQPLSTPLSHSTYV

SEQ ID NO: 480 - protein, 412 aa, Artificial Sequence
  synthetic sequence
NVKYGIVLDAGSSHTSLYIYKWPAEKENDTGVVHQVECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLSQGYHFTADSWEHIHFIGKIQGSDAGWTLGYMLNLTNMIPAEQPLSTPLSHSTYV

SEQ ID NO: 481 - protein, 434 aa, Artificial Sequence
  synthetic sequence
TRDVREPPALKYGIVLDAGSSHTSMFIYKWPADKENDTGIVGQHSSCDVPGGGISSYADNPSGASQSLVGCLEQALQDVPKERHAGTPLYLGATAGMRLLNLTNPEASTSVLMAVTHTLTQYPFDFRGARILSGQEEGVFGWVTANYLLENFIKYGWVGRWFRPRKGTLGAMDLGGASTQITFETTSPAEDRASEVQLHLYGQHYRVYTHSFLCYGRDQVLQRLLASALQTHGFHPCWPRGFSTQVLLGDVYQSPCTMAQRPQNFNSSARVSLSGSSDPHLCRDLVSGLFSFSSCPFSRCSFNGVFQPPVAGNFVAFSAFFYTVDFLRTSMGLPVATLQQLEAAAVNVCNQTWAQLQARVPGQRARLADYCAGAMFVQQLLSRGYGFDERAFGGVIFQKKAADTAVGWALGYMLNLTNLIPADPPGLRKGTDFS

SEQ ID NO: 482 - protein, 442 aa, Artificial Sequence
  synthetic sequence
QIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPP

SEQ ID NO: 483 - protein, 443 aa, Artificial Sequence
  synthetic sequence
KVEATSVLLPTDIKFGIVFDAGSSHTSLFLYQWLANKENGTGVVSQALACQVEGPGISSYTSNAAQAGESLQGCLEEALVLIPEAQHRKTPTFLGATAGMRLLSRKNSSQARDIFAAVTQVLGRSPVDFWGAELLAGQAEGAFGWITVNYGLGTLVKYSFTGEWIQPPEEMLVGALDMGGASTQITFVPGGPILDKSTQADFRLYGSDYSVYTHSYLCFGRDQMLSRLLVGLVQSRPAALLRHPCYLSGYQTTLALGPLYESPCVHATPPLSLPQNLTVEGTGNPGACVSAIRELFNFSSCQGQEDCAFDGVYQPPLRGQFYAFSNFYYTFHFLNLTSRQPLSTVNATIWEFCQRPWKLVEASYPGQDRWLRDYCASGLYILTLLHEGYGFSEETWPSLEFRKQAGGVDIGWTLGYMLNLTGMIPADAPAQWRAESYGVWVAK

SEQ ID NO: 484 - protein, 10 aa, Artificial Sequence
  synthetic sequence
GGGGSGGGGS

(19) United States
(12) Patent Application Publication
(10) Pub. No.: US 2025/0019431 A1
(43) Pub. Date: Jan. 16, 2025

(54) KIDNEY TARGETED IMMUNOTOLERANCE

(71) Applicant: PANDION OPERATIONS, INC., Watertown, MA (US)

(72) Inventors: Bridget LARKIN, Watertown, MA (US); Bilian LI, Watertown, MA (US); Nathan HIGGINSON-SCOTT, Hineham, MA (US); Kevin Lewis OTIPOBY, Ashland, MA (US); Joanne L. VINEY, Belmont, MA (US)

(73) Assignee: PANDION OPERATIONS, INC., Watertown, MA (US)

(21) Appl. No.: 18/248,398

(22) PCT Filed: Oct. 15, 2021
(86) PCT No.: PCT/US2021/055245

§ 371 Date: Apr. 10, 2023

Related U.S. Application Data
(60) Provisional application No. 63/175,857, filed on Apr. 16, 2021.
(60) Provisional application No. 63/156,053, filed on Mar. 3, 2021.
(60) Provisional application No. 63/092,793, filed on Oct. 16, 2020.

(51) Int. Cl. C07K 16/28; C07K 14/55; C07K 14/705; C07K 16/24

(52) U.S. Cl. CPC C07K 16/28; C07K 14/55; C07K 14/70596; C07K 16/24; C07K 2317/31; C07K 2317/622; C07K 2319/03; C07K 2319/30; C07K 2319/33

(57) ABSTRACT

Methods and compounds for conferring kidney-specific immune privilege.

### CROSS-REFERENCE TO RELATED APPLICATIONS

This application claims priority to U.S. Provisional Application No. 63/175,857, filed Apr. 16, 2021, U.S. Provisional Application No. 63/156,053, filed Mar. 3, 2021, U.S. Provisional Application No. 63/092,793, filed Oct. 16, 2020, each of which is hereby incorporated by reference in its entirety.

### FIELD

The embodiments provided herein relate to, for example, methods and compositions for kidney-targeted immune privilege.

### BACKGROUND

Instances of unwanted immune responses, e.g., as in the rejection of transplanted tissue or in autoimmune disorders, constitute a major health problem for millions of people across the world. Long-term outcomes for organ transplantation are frequently characterized by chronic rejection, and eventual failure of the transplanted organ. More than twenty autoimmune disorders are known, affecting essentially every organ of the body, and affecting over fifty million people in North America alone. The broadly active immunosuppressive medications used to combat the pathogenic immune response in both scenarios have serious side effects.

### SUMMARY

In some embodiments, polypeptides comprising a kidney targeting moiety that binds to a target kidney cell and an effector binding/modulating moiety, wherein the effector binding/modulating moiety can be, but is not limited to, a PD-1 agonist, CD39 Effector Domain, or an IL-2 mutein polypeptide (IL-2 mutein).

In some embodiments, methods of treating a subject with a kidney disorder, such as, but not limited to, Goodpasture's Syndrome (anti-GBM disease), inflammatory renal disease, glomerulonephritis, nephritis, lupus, lupus nephritis, IgA nephritis, membranous nephropathy, membranoproliferative glomerulonephritis, acute kidney injury, and chronic kidney disease, focal segmented glomerular sclerosis (FSGS), lupus nephritis, systemic scleroderma, membranous glomerular nephropathy (MGN), membranous nephropathy (MN), minimal change disease (MCD), IgA nephropathy, ANCA-associated vasculitis (AAV), Sjogren's syndrome, and Scleroderma, systemic sclerosis (SSc), graft versus host disease (GVHD) of a kidney transplant, and the like are provided. In some embodiments, the methods comprise administering a polypeptide, compound, or composition as provided herein to the subject to treat the disorder.

In some embodiments, methods of treating GVHD are provided, wherein the method comprises administering polypeptide, compound, or composition as provided herein to a subject to treat the GVHD. In some embodiments, the GVHD is GVHD of the kidney.

In some embodiments, methods of treating a subject who has had a transplant, such as a kidney transplant, subject are provided. In some embodiments, the methods comprise comprising administering a therapeutically effective amount of a polypeptide, compound, or composition as provided herein to the subject, thereby treating the transplant (recipient) subject.

In some embodiments, methods of treating GVHD in a subject having a transplanted a donor tissue (e.g. kidney tissue) are provided. In some embodiments, the methods of treating GVHD in a subject having a transplanted a donor tissue (e.g. kidney tissue) comprise administering a therapeutically effective amount of a polypeptide, compound, or composition as provided herein to the subject.

In some embodiments, pharmaceutical composition comprising polypeptide, compound, or composition as provided herein are provided.

Also disclosed herein are methods and therapeutic compounds that provide site-specific immune privilege, such as at the kidney. Embodiments disclosed herein are incorporated by reference into this section.

### BRIEF DESCRIPTION OF THE DRAWINGS

FIG. 1 depicts staining of bi-functional molecules comprising an anti-CDH16 antibody and IL-2 mutein staining in the tubules.

FIG. 2 depicts staining of bi-functional molecules comprising an anti-CDH16 antibody and IL-2 mutein staining in the kidney ex-vivo.

FIG. 3 depicts a non-limiting illustration of the therapeutic compounds provided herein.

FIG. 3A depicts a non-limiting illustration of the therapeutic compounds provided herein.

FIG. 4 depicts a non-limiting illustration of the therapeutic compounds provided herein.

FIG. 5 depicts a non-limiting illustration of the therapeutic compounds provided herein.

FIG. 6 depicts a non-limiting illustration of the therapeutic compounds provided herein.

FIG. 7 depicts a non-limiting illustration of the therapeutic compounds provided herein.

FIG. 8 depicts a non-limiting illustration of the therapeutic compounds provided herein.

FIG. 9 depicts a non-limiting illustration of the therapeutic compounds provided herein.

FIG. 10 depicts a non-limiting illustration of the therapeutic compounds provided herein.

FIG. 11 depicts a non-limiting illustration of the therapeutic compounds provided herein.

FIG. 12 depicts a non-limiting illustration of the therapeutic compounds provided herein.

FIG. 13 depicts a non-limiting illustration of the therapeutic compounds provided herein.

FIG. 14 depicts a non-limiting illustration of the therapeutic compounds provided herein.

FIG. 15 depicts a non-limiting illustration of the therapeutic compounds provided herein.

FIG. 16 depicts a non-limiting illustration of the therapeutic compounds provided herein.

FIG. 17 depicts a non-limiting illustration of the therapeutic compounds provided herein.

FIG. 18 depicts a non-limiting illustration of the therapeutic compounds provided herein.

FIG. 19 depicts a non-limiting illustration of the therapeutic compounds provided herein.

FIG. 20 depicts a non-limiting illustration of the therapeutic compounds provided herein.

### DETAILED DESCRIPTION

This application incorporates by reference each of the following in its entirety: U.S. application Ser. No. 15/922,592 filed Mar. 15, 2018 and PCT Application No. PCT/US2018/022675, filed Mar. 15, 2018. This application also incorporate by reference, each of the following in their entirety: U.S. Provisional Application No. 63/029,793, filed Oct. 16, 2020, U.S. Provisional Application No. 62/888,694, filed Aug. 19, 2020, U.S. Provisional Application No. 62/850,172, filed May 20, 2019, U.S. Provisional Application No. 62/721,644, filed Aug. 23, 2018, U.S. provisional Application No. 62/675,972 filed May 24, 2018, U.S. provisional Application No. 62/595,357 filed Dec. 6, 2017, U.S. Provisional Application No. 62/595,348, filed Dec. 6, 2017, U.S. Non-Provisional application Ser. No. 16/109,875, filed Aug. 23, 2018, U.S. Non-Provisional application Ser. No. 16/109,897, filed Aug. 23, 2018, U.S. Non-Provisional application Ser. No. 15/988,311, filed May 24, 2018, PCT Application No. PCT/US2018/034334, filed May 24, 2018, and, PCT/US2018/062780, filed Nov. 28, 2018.

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±5% and remain within the scope of the disclosed embodiments.

As used herein, the term “animal” includes, but is not limited to, humans and non-human vertebrates such as wild, domestic, and farm animals. In some embodiments, the animal is a mammal. The term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or via a linker sequence, such as the glycine/serine sequences described herein to link the two domains together.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. A result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

In some embodiments, therapeutic compounds are provided herein. In some embodiments, the therapeutic compound is a protein or a polypeptide, that has multiple peptide chains that interact with one another. The polypeptides can interact with one another through non-covalent interactions or covalent interactions, such as through disulfide bonds or other covalent bonds. Therefore, if an embodiment refers to a therapeutic compound it can also be said to refer to a protein or polypeptide as provided for herein and vice versa as the context dictates.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10^{-4M}, at least about 10⁻⁵M, at least about 10⁻⁶M, at least about 10⁻⁷M, at least about 10⁻⁸M, at least about 10⁻⁹M, alternatively at least about 10⁻¹⁰ M, at least about 10⁻¹¹M, at least about 10⁻¹²M, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

As provided herein, the therapeutic compounds and compositions can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of an autoimmune disease/disorder” means an activity that alleviates or ameliorates any of the primary phenomena or secondary symptoms associated with the autoimmune disease/disorder or other condition described herein. The various disease or conditions are provided herein. The therapeutic treatment can also be administered prophylactically to preventing or reduce the disease or condition before the onset.

### PD-1 Agonists

Provided herein are therapeutic compounds, e.g., therapeutic polypeptide molecules, such as fusion proteins, including a targeting moiety and an effector binding/modulating moiety, typically as separate domains. In some embodiments, the therapeutic compound comprises a polypeptide as provided herein. In some embodiments, the therapeutic compound comprises a polypeptide complex as provided herein. Also provided are methods of using and making the therapeutic compounds. The targeting moiety serves to localize the therapeutic compound, and thus the effector binding/modulating moiety, to a site at which immune privilege is desired. As used herein, “immune privilege” means lack of, or suppression of an inflammatory response. As a non-limiting example, immune privilege includes situations where a tissue or site in the body is able to tolerate the introduction of antigens without eliciting an inflammatory immune response (Forester J. V., Lambe H. Xu, Cornall R. Immune Previlege or privileged immunity? Mucosal Immunology, 1, 372-381 (2008)).

The present disclosure provides, for example, effector molecules that can act as PD-1 agonists. In some embodiments, the agonist is an antibody that binds to and agonizes PD-1. Without wishing to be bound to any particular theory, agonism of PD-1 inhibits T cell activation/signaling and can be accomplished by different mechanisms. For example crosslinking of bead-bound functional PD-1 agonists can lead to agonism. Functional PD-1 agonists have been described (Akkaya. Ph.D. Thesis: Modulation of the PD-1 pathway by inhibitory antibody superagonists. Christ Church College, Oxford, UK, 2012), which is hereby incorporated by reference. Crosslinking of PD-1 with two mAbs that bind non-overlapping epitopes induces PD-1 signaling (Davis, US 2011/0171220), which is hereby incorporated by reference. Another example is illustrated through the use of a goat anti-PD-1 antiserum (e.g. AF1086, R&D Systems) which is hereby incorporated by reference, which acts as an agonist when soluble (Said et al., 2010, Nat Med) which is hereby incorporated by reference. Non-limiting examples of PD-1 agonists that can be used in the present embodiments include, but are not limited to, UCB clone 19 or clone 10, PD1AB-1, PD1AB-2, PD1AB-3, PD1AB-4 and PD1AB-5, PD1AB-6 (Anaptys/Celgene), PD1-17, PD1-28, PD1-33 and PD1-35 (Collins et al, US 2008/0311117 A1), antibodies against PD-1 and uses therefor, which is hereby incorporated by reference, or can be a bispecific, monovalent anti-PD-1/anti-CD3 (Ono), and the like. In some embodiments, the PD-1 agonist antibodies can be antibodies that block binding of PD-L1 to PD-1. In some embodiments, the PD-1 agonist antibodies can be antibodies that do not block binding of PD-L1 to PD-1. In some embodiments, the antibody does not act as an antagonist of PD-1.

PD-1 agonism can be measured by any method, such as the methods described in the examples. For example, cells can be constructed that express, including stably express, constructs that include a human PD-1 polypeptide fused to a beta-galactosidase “Enzyme donor” and 2) a SHP-2 polypeptide fused to a beta-galactosidase “Enzyme acceptor.” Without being bound by any theory, when PD-1 is engaged, SHP-2 is recruited to PD-1. The enzyme acceptor and enzyme donor form a fully active beta-galactosidase enzyme that can be assayed. Although, the assay does not directly show PD-1 agonism, but shows activation of PD-1 signaling. PD-1 agonism can also be measured by measuring inhibition of T cell activation because, without being bound to any theory, PD-1 agonism inhibits anti-CD3-induced T cell activation. For example, PD-1 agonism can be measured by preactivating T cells with PHA (for human T cells) or ConA (for mouse T cells) so that they express PD-1. The cells can then be reactivated with anti-CD3 in the presence of anti-PD-1 (or PD-L1) for the PD-1 agonism assay. T cells that receive a PD-1 agonist signal in the presence of anti-CD3 will show decreased activation, relative to anti-CD3 stimulation alone. Activation can be readout by proliferation or cytokine production (IL-2, IFNg, IL-17) or other markers, such as CD69 activation marker. Thus, PD-1 agonism can be measured by either cytokine production or cell proliferation. Other methods can also be used to measure PD-1 agonism.

PD-1 is an Ig superfamily member expressed on activated T cells and other immune cells. The natural ligands for PD-1 appear to be PD-L1 and PD-L2. Without being bound to any particular theory, when PD-L1 or PD-L2 bind to PD-1 on an activated T cell, an inhibitory signaling cascade is initiated, resulting in attenuation of the activated T effector cell function. Thus, blocking the interaction between PD-1 on a T cell, and PD-L1/2 on another cell (e.g., tumor cell) with a PD-1 antagonist is known as checkpoint inhibition, and releases the T cells from inhibition. In contrast, PD-1 agonist antibodies can bind to PD-1 and send an inhibitory signal and attenuate the function of a T cell. Thus, PD-1 agonist antibodies can be incorporated into various embodiments described herein as an effector molecule binding/modulating moiety (sometimes also referred to herein as an effector molecule), which can accomplish localized tissue-specific immunomodulation when paired with a targeting moiety.

The effector molecules, including the PD-1 agonist, can be linked to a targeting, moiety, such as one that binds to CDH16. As used herein, the term “CDH16” refers to the protein Cadherin 16, which can also be referred to as KSP-Cadherin, Kidney-Specific Cadherin, or even CDH16. In some embodiments, the effector molecules can be linked to a targeting moiety such as one that binds to OCT2. As used herein, the rem “OCT2” refers to the protein organic cation transporter-2, which can also be referred to as SLC22A2, or solute carrier family 22 member 2.

In some embodiments, the targeting moiety (e.g., that binds to CDH16 or to OCT2) and effector binding/modulating moiety (e.g. PD-1 agonist, CD39 effector domain, and/or IL-2 mutein) are physically tethered, covalently or non-covalently, directly or through a linker entity, to one another, e.g., as a member of the same protein molecule in a therapeutic protein molecule. In some embodiments, the targeting and effector moieties are provided in a therapeutic protein molecule, e.g., a fusion protein, typically as separate domains. In some embodiments, the targeting moiety, the effector binding/modulating moiety, or both each comprises a single domain antibody molecule, e.g., a camelid antibody VHH molecule or human soluble VH domain. It may also contain a single-chain fragment variable (scFv) or a Fab domain. In some embodiments, the therapeutic protein molecule, or a nucleic acid, e.g., an mRNA or DNA, encoding the therapeutic protein molecule, can be administered to a subject. In some embodiments, the targeting and effector molecule binding/modulating moieties are linked to a third entity, e.g., a carrier, e.g., a polymeric carrier, a dendrimer, or a particle, e.g., a nanoparticle. The therapeutic compounds can be used to down regulate an immune response at or in a tissue at a selected target or site while having no or substantially less immunosuppressive function systemically. The target or site can comprise donor tissue or autologous tissue.

Provided herein are methods of providing site-specific immune privilege for a transplanted donor tissue, e.g., an allograft tissue, e.g., a tissue described herein, e.g., an allograft kidney, with the therapeutic compounds (e.g. polypeptides) provided for herein. In some embodiments, the treatment minimizes rejection of, minimizes immune effector cell mediated damage to, prolongs acceptance of, or prolongs the functional life of, donor transplant tissue. In some embodiments, the methods comprise administering effective amount of a polypeptide as provided for herein to the subject to inhibit rejection of an allograft tissue, such as a kidney that is transplanted into the subject.

In some embodiments, the subject that is treated with the polypeptides provided for herein are characterized as having end renal stage disease or are in need of a kidney transplant.

Also provided herein are methods of inhibiting GVHD by minimizing the ability of donor immune cells, e.g., donor T cells, to mediate immune attack of recipient tissue, with therapeutic compounds disclosed herein. In some embodiments, the methods comprise administering an effective amount of a polypeptide as provided for herein to the subject to inhibit GVHD in a subject who has had a kidney transplant.

Also provided herein are methods of treating, e.g., therapeutically treating or prophylactically treating (or preventing), an autoimmune disorder or autoimmune response in a subject by administration of an effective amount of a therapeutic compound disclosed herein, e.g., to provide site or tissue specific modulation of the immune system. Examples of autoimmune diseases or inflammation that impact the kidney that can be treated include, but are not limited to Goodpasture's Syndrome (anti-GBM disease), inflammatory renal disease, glomerulonephritis, nephritis, lupus, lupus nephritis, IgA nephritis, membranous nephropathy, membranoproliferative glomerulonephritis, acute kidney injury, and chronic kidney disease as well as any other autoimmune or inflammation disorders that can affect the kidneys.

In some embodiments, administration of the therapeutic compound, which can also be referred to as a polypeptide throughout the present specification, begins after the disorder is apparent. In some embodiments, administration of the therapeutic compound begins prior to onset, or full onset, of the disorder, e.g., in a subject having the disorder, a high-risk subject, a subject having a biomarker for risk or presence of the disorder, a subject having a family history of the disorder, or other indicator of risk of, or asymptomatic presence of, the disorder. For example, in some embodiments, a subject having islet cell damage but which is not yet diabetic, is treated.

While not wishing to be bound by theory, it is believed that the targeting moiety functions to bind and accumulate the therapeutic compound to a target selectively or preferentially expressed at the anatomical site where immune privilege is desired. In some embodiments, e.g., in the context of donor tissue transplantation, the target moiety binds to a target, e.g., an allelic product, present in the donor tissue but not the recipient. For treatment of autoimmune disorders, the targeting moiety binds a target preferentially expressed at the anatomical site where immune privilege is desired, e.g., in the kidney. For treatment of GVHD, the targeting moiety targets the host tissue, and protects the host against attack from transplanted immune effector cells derived from transplanted tissue.

Again, while not wishing to be bound by theory, it is believed that the effector binding/modulating moiety serves to deliver an immunosuppressive signal or otherwise create an immune privileged environment.

As used herein, effector, or effector moiety, refers to an entity, e.g., a cell or molecule, e.g., a soluble or cell surface molecule, which mediates an immune response. Non-limiting examples of effector molecules are PD-1 agonists, IL-2 muteins, and the CD39 domains and polypeptides, such as those provided for herein.

Effector ligand binding molecule, or effector ligand binding moiety, as used herein, refers to a polypeptide that has sufficient sequence from a naturally occurring counter ligand of an effector, that it can bind the effector with sufficient specificity that it can serve as an effector binding/modulating molecule. In some embodiments, an effector ligand binding molecule binds to effector with at least 10, 20, 30, 40, 50, 60, 70, 80, 90, or 95% of the affinity of the naturally occurring counter ligand. In some embodiments, an effector ligand binding molecule has at least 60, 70, 80, 90, 95, 99, or 100% sequence identity, or substantial sequence identity, with a naturally occurring counter ligand for the effector.

Effector specific binding polypeptide, or effector specific binding moiety, as used herein, refers to a polypeptide that can bind with sufficient specificity that it can serve as an effector binding/modulating moiety. In some embodiments, a specific binding polypeptide comprises a effector ligand binding molecule.

Unless otherwise defined, all technical and scientific terms used herein have the same meaning as commonly understood by one of ordinary skill in the art to which these embodiments belong. Although methods and materials similar or equivalent to those described herein can be used in the practice or testing of the present embodiments, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the embodiments will be apparent from the description and drawings, and from the claims.

### Additional Definitions

Unless defined otherwise, all technical and scientific terms used herein have the same meaning as commonly understood by one of ordinary skill in the art to which the embodiments pertain. In describing and claiming the present embodiments, the following terminology and terminology otherwise referenced throughout the present application will be used according to how it is defined, where a definition is provided.

It is also to be understood that the terminology used herein is for the purpose of describing particular embodiments only, and is not intended to be limiting.

Antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen binding portion of an immunoglobulin molecule, such as an antibody fragment. An antibody fragment, e.g., functional fragment, comprises a portion of an antibody, e.g., Fab, Fab′, F(ab′)2, F (ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full-length antibodies and antibody fragments, e.g., dAb (domain antibody), single chain, Fab, Fab′, and F(ab′)2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, or hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

Examples of formats for multispecific therapeutic compounds, e.g., bispecific antibody molecules are shown in the following non-limiting examples. Although illustrated with antibody molecules, they can be used as platforms for therapeutic molecules that include other non-antibody moieties as specific binding or effector moieties. In some embodiments, these non-limiting examples are based upon either a symmetrical or asymmetrical Fc formats.

For example, the figures illustrate non-limiting and varied symmetric homodimer approach. In some embodiments, the dimerization interface centers around human IgG1 CH2-CH3 domains, which dimerize via a contact interface spanning both CH2/CH2 and CH3/CH3. The resulting bispecific antibodies shown have a total valence comprised of four binding units with two identical binding units at the N-terminus on each side of the dimer and two identical units at the C-terminus on each side of the dimer. In each case the binding units at the N-terminus of the homodimer are different from those at the C-terminus of the homodimer. Using this type of bivalency for both an inhibitory T cell receptor at either terminus of the molecule and bivalency for a tissue tethering antigen can be achieved at either end of the molecule.

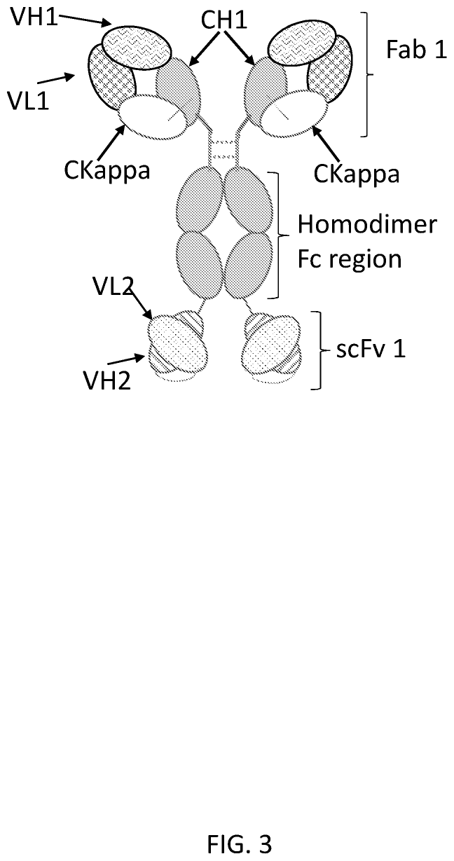

For example, in FIG. 3, a non-limiting embodiment is illustrated. The N-terminus of the homodimer contains two identical Fab domains comprised of two identical light chains, which are separate polypeptides, interfaced with the n-terminal VH-CH1 domains of each heavy chain via the VH/VL interaction and Ckappa or Clambda interaction with CH1. The native disulfide bond between the Ckappa or Clambda with CH1 provides a covalent anchor between the light and heavy chains. At the C-terminus of this design are two identical scFv units where by (in this example) the C-terminus of the CH3 domain of the Fc, is followed by a flexible, hydrophilic linker typically comprised of (but not limited to) serine, glycine, alanine, and/or threonine residues, which is followed by the VH domain of each scFv unit, which is followed by a glycine/serine rich linker, followed by a VL domain. These tandem VH and VL domains associate to form a single chain fragment variable (scFv) appended at the C-terminus of the Fc. Two such units exist at the C-terminus of this molecule owing to the homodimeric nature centered at the Fc. The domain order of scFvs may be configured to be from N- to C-terminus either VH-Linker-VL or VL-Linker-VH.

A non-limiting example of a molecule that has different binding regions on the different ends is where, one end is a PD-1 agonist and the antibody that provides target specificity is an anti-CDH16 antibody. This can be illustrated as shown, for example, in FIG. 3A, which illustrates the molecules in different orientations.

In some embodiments, the PD-1 agonist is replaced with an IL-2 mutein, such as, but not limited to, the ones described herein.

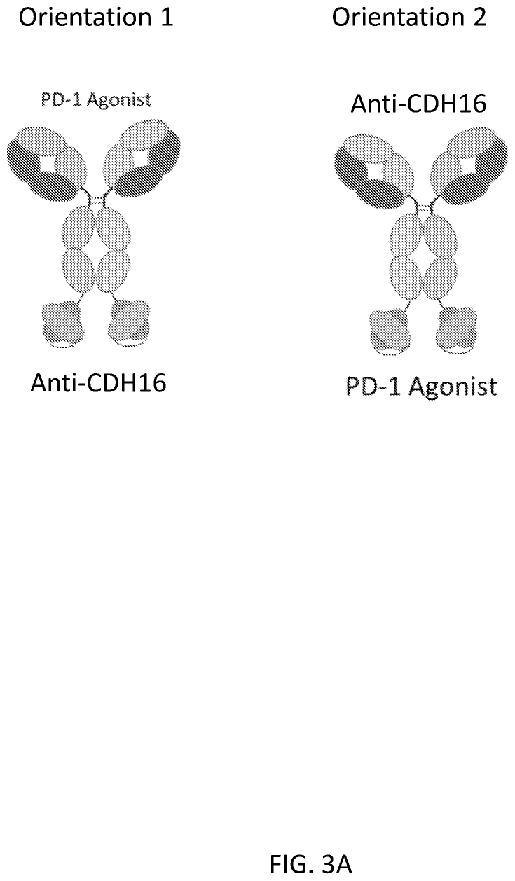

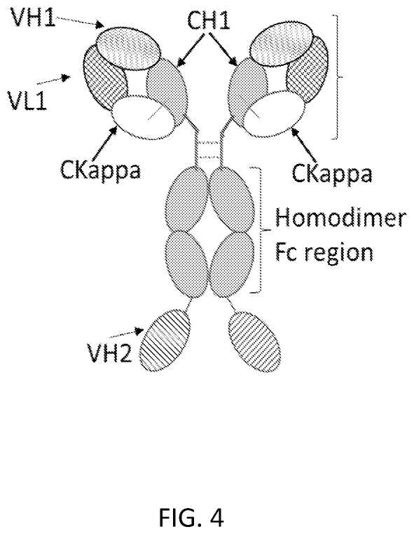

In another example, and as depicted in FIG. 4, the N-terminus of the homodimer contains two identical Fab domains comprised of two identical light chains, which are separate polypeptides, interfaced with the N-terminal VH-CH1 domains of each heavy chain via the VH/VL interaction and Ckappa or Clambda interaction with CH1. The native disulfide bond between the Ckappa or Clambda with CH1 provides a covalent anchor between the light and heavy chains. At the C-terminus of this design are two identical VH units (though non-antibody moieties could also be substituted here or at any of the four terminal attachment/fusion points) where by (in this example) the C-terminus of the CH3 domain of the Fc, is followed by a flexible, hydrophilic linker typically comprised of (but not limited to) serine, glycine, alanine, and/or threonine residues, which is followed by a soluble independent VH3 germline family based VH domain. Two such units exist at the C-terminus of this molecule owing to the homodimeric nature centered at the Fc.

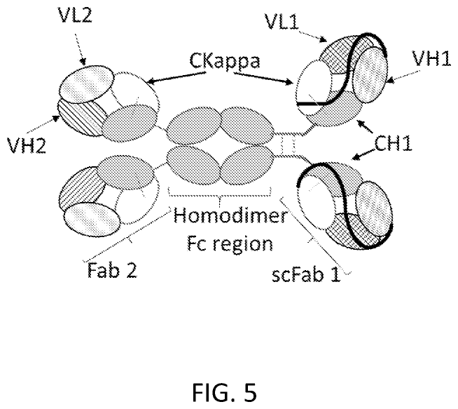

In another non-limiting example, as depicted in FIG. 5, the N-terminus of the homodimer contains two identical Fab domains comprised of two identical light chains, which, unlike FIG. 3 and FIG. 4, are physically conjoined with the heavy chain at the N-terminus via a linker between the C-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 provides additional stability between the light and heavy chains. At the C-terminus of this design are two identical Fab units where by (in this example) the C-terminus of the CH3 domain of the Fc, is followed by a flexible, hydrophilic linker typically comprised of (but not limited to) serine, glycine, alanine, and/or threonine residues, which is followed by a CH1 domain, followed by a VH domain at the C-terminus. The light chain that is designed to pair with the C-terminal CH1/VH domains is expressed as a separate polypeptide, unlike the N-terminal light chain which is conjoined to the N-terminal VH/CH1 domains as described. The C-terminal light chains form an interface at between VH/VL and Ckappa or Clambda with CH1. The native disulfide anchors this light chain to the heavy chain. Again, any of the antibody moieties at any of the four attachment/fusion points can be substituted with a non-antibody moiety, e.g., an effector binding/modulating moiety that does not comprise an antibody molecule.

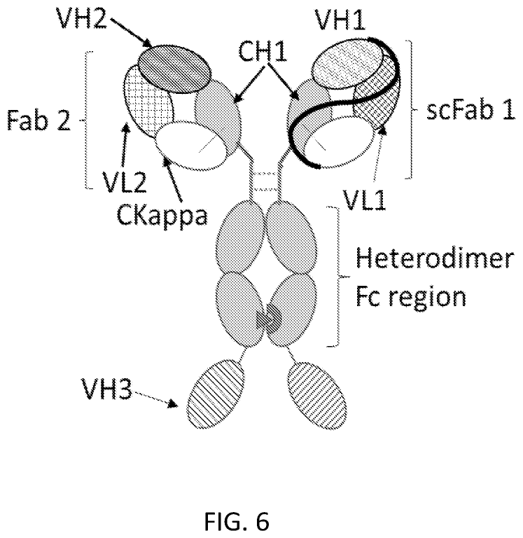

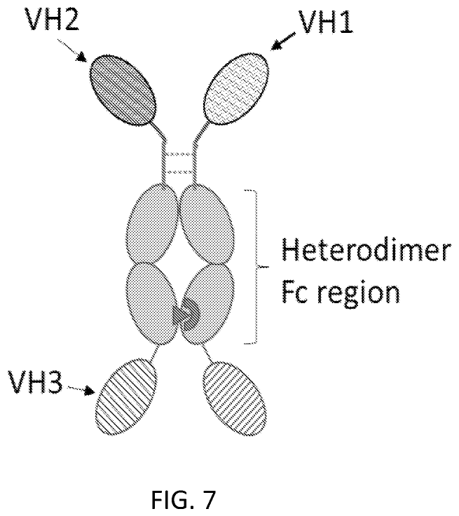

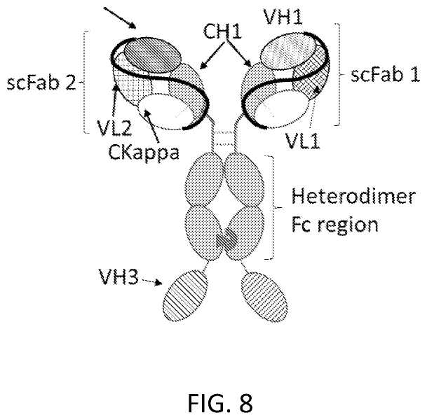

The bispecific antibodies can also be asymmetric as shown in the following non-limiting examples. Non-limiting example are also depicted in FIG. 6, FIG. 7, and FIG. 8, which illustrate an asymmetric/heterodimer approach. Again, in any of these formats, any of the antibody moieties at any of the four attachment/fusion points can be substituted with a non-antibody moiety, e.g., a effector binding/modulating moiety that does not comprise an antibody molecule. In some embodiments, the dimerization interface centers around the human IgG1 CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (Kabat) in one CH3 domain and T366S, L368A, and Y407V (Kabat) mutations in the other CH3 domain. The heterodimerizing interface may be further stabilized with de novo disulfide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4. In the illustrative examples below, the C-terminus comprises two identical binding domains which could, for example, provide bivalent monospecificity for a tissue tethering target. At the N-terminus of all three of the illustrative examples, both binding domains comprise different recognition elements/paratopes, which could achieve recognition of two different epitopes on the same effector moiety target, or could recognize for example a T cell inhibitory receptor and CD3. In some embodiments, the N-terminal binding moieties may be interchanged with other single polypeptide formats such as scFv, single chain Fab, tandem scFv, VH or VHH domain antibody configurations for example. Other types of recognition element may be used also, such as linear or cyclic peptides.

An example of an asymmetric molecule is depicted in FIG. 6. Referring to FIG. 6, the N-terminus of the molecule is comprised of a first light chain paired with a first heavy chain via VH/VL and Ckappa or Clambda/CH1 interactions and a covalent tether comprised of the native heavy/light chain disulfide bond. On the opposite side of this heterodimeric molecule at the N-terminus is a second light chain and a second heavy chain which are physically conjoined via a linker between the C-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 provides additional stability between the light and heavy chains. At the C-terminus of the molecule are two identical soluble VH3 germline family VH domains joined via an N-terminal glycine/serine/alanine/threonine based linker to the C-terminus of the CH3 domain of both heavy chain 1 and heavy chain 2.

In some embodiments, an asymmetric molecule can be as illustrated as depicted in FIG. 7. For example, the N-terminus of the molecule is comprised of two different VH3 germlined based soluble VH domains linked to the human IgG1 hinge region via a glycine/serine/alanine/threonine based linker. The VH domain connected to the first heavy chain is different to the VH domain connected to the second heavy chain. At the C-terminus of each heavy chain is an additional soluble VH3 germline based VH domain, which is identical on each of the two heavy chains. The heavy chain heterodimerizes via the previously described knobs into holes mutations present at the CH3 interface of the Fc module.

In some embodiments, an asymmetric molecule can be as illustrated in FIG. 8. This example is similar to the molecule shown in FIG. 7, except both N-terminal Fab units are configured in a way that light chain 1 and light chain 2 are physically conjoined with heavy chain 1 and heavy chain 2 via a linker between the C-terminus of Ckappa or Clambda and the N-terminus of each respective VH. The linker in each case may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 provides additional stability between the light and heavy chains.

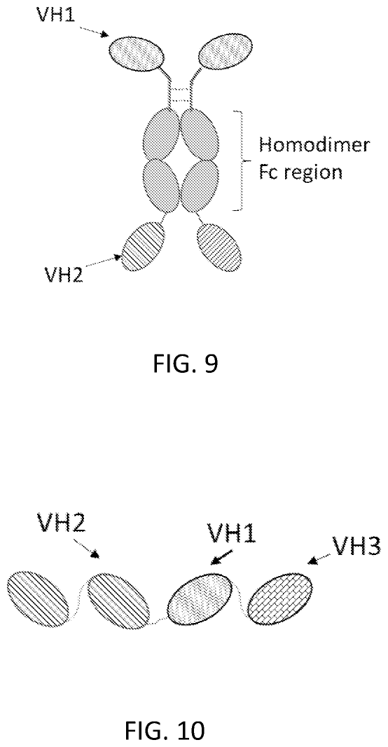

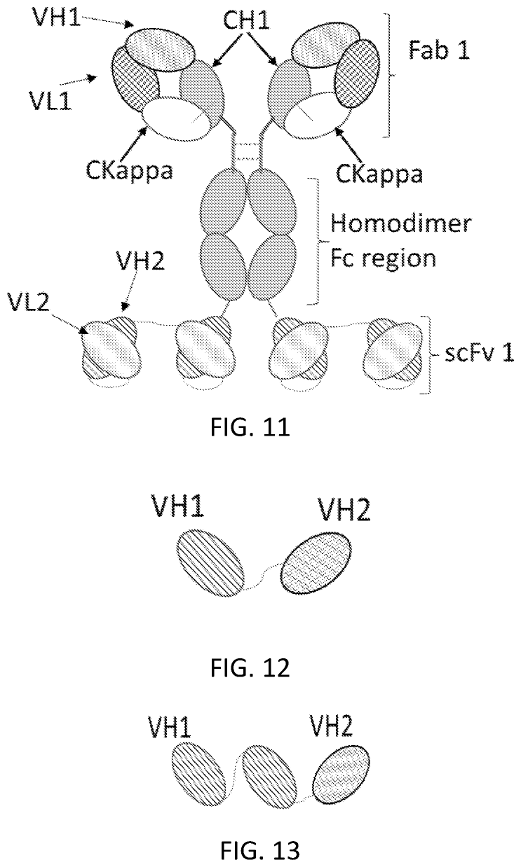

Bispecific molecules can also have a mixed format. This is illustrated, for example, in FIG. 9, FIG. 10, and FIG. 11.

For example, FIG. 9, illustrates a homodimer Fc based approach (see FIGS. 3, 4, and 5), combined with the moiety format selection of FIG. 7, whereby the total molecular valency is four, but specificity is restricted to two specificities. The N-terminus is comprised of two identical soluble VH3 germline based VH domains and the C-terminus is comprised of two identical soluble VH3 germlined based VH domains of different specificity to the N-terminal domains. Therefore, each specificity has a valence of two. Again, in this format, any of the antibody moieties at any of the four attachment/fusion points can be substituted with a non-antibody moiety, e.g., an effector binding/modulating moiety that does not comprise an antibody molecule.

FIG. 10 illustrates an example whereby the molecule is comprised of four VH3 germline based soluble VH domains. The first two domains have the same specificity (for example an inhibitory receptor), the 3rd domain from the N-terminus may have specificity for a tissue antigen and the fourth domain from the N-terminus may have specificity for human serum albumin (HSA), thereby granting the molecule extended half-life in the absence of an Ig Fc domain. Three glycine, serine, alanine and/or threonine rich linkers exists between domains 1 and 2, domains 2 and 3, and domains 3 and 4. This format may be configured with up to tetraspecificity, but monovalent in each case, or to have bispecificity with bivalency in each case. The order of domains can be changed. Again, in this format, any of the antibody moieties can be substituted with a non-antibody moiety, e.g., a effector binding/modulating moiety that does not comprise an antibody molecule.

FIG. 11 illustrates yet another approach. This example is similar to FIGS. 3 and 4, in that it is Fc homodimer based with two identical Fab units (bivalent monospecificity) at the N-terminus of the molecule. This example differs from FIGS. 3 and 4 in that the C-terminus of each heavy chain is appended with a tandem-scFv. Thus, in each case the C-terminus of the CH3 domain of the Fc is linked via a glycine/serine/alanine/threonine based linker to the N-terminus of a first VH domain, which is linked via the C-terminus by a 12-15 amino acid glycine/serine rich linker to the N-terminus of a first VL domain, which linked via a 25-35 amino acid glycine/serine/alanine/threonine based linker at the C-terminus to the N-terminus of a second VH domain, which is linked via the C-terminus with a 12-15 amino acid glycine/serine based linker to the N-terminus of a 2nd VL domain. In this Fc homodimer based molecule there are therefore two identical tandem scFvs at the C-terminus of the molecule offering either tetravalency for a single tissue antigen for example or bivalency to two different molecules. This format could also be adapted with a heterodimer Fc core allowing two different tandem-scFvs at the C-terminus of the Fc allowing for monovalent tetraspecificity at the C-terminus while retaining either bivalent monospecificity at the N-terminus or monovalent bispecificity at the N-terminal via usage of single chain Fab configurations as in FIGS. 5, 6, and 7. This molecule can therefore be configured to have 2, 3, 4, 5, or 6 specificities. The domain order of scFvs within the tandem-scFv units may be configured to be from N- to C-terminus either VH-Linker-VL or VL-Linker-VH. Again, in this format, any of the antibody moieties at any of the four attachment/fusion points can be substituted with a non-antibody moiety, e.g., an effector binding/modulating moiety that does not comprise an antibody molecule.

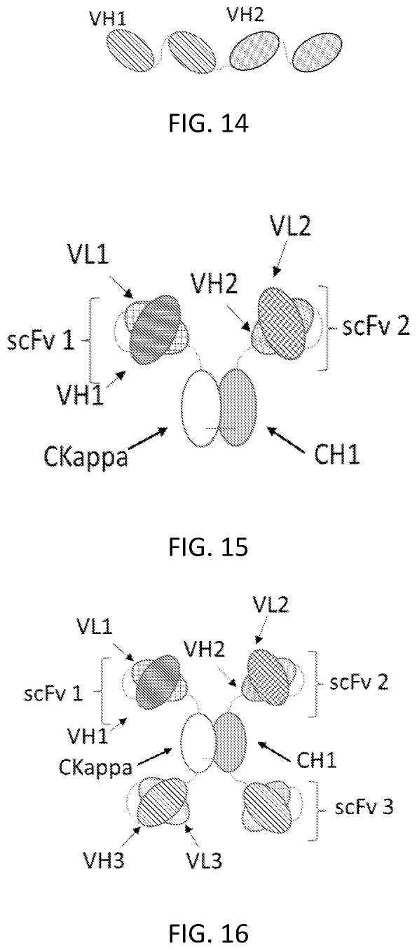

Bispecific antibodies can also be constructed to have, for example, shorter systemic PK while having increased tissue penetration. These types of antibodies can be based upon, for example, a human VH3 based domain antibody format. These are illustrated, for example, in FIGS. 12, 13, and 14. FIGS. 12, 13, and 14 each comprised a soluble VH3 germline family based VH domain modules. Each domain is approximately 12.5 kDa allowing for a small overall MW, which, without being bound to any particular theory, should be beneficial for enhanced tissue penetration. In these examples, none of the VH domains recognize any half-life extending targets such as FcRn or HSA. As illustrated in FIG. 12, the molecule is comprised of two VH domains joined with a flexible hydrophilic glycine/serine based linker between the C-terminus of the first domain and N-terminus of the second domain. In this example one domain may recognize a T cell costimulatory receptor and the second may recognize a tissue tethering antigen. As illustrated in FIG. 13, the molecule is comprised of three VH domains with N—C-terminal linkages of hydrophilic glycine/serine based linkers. The molecule may be configured to be trispecific but monovalent for each target. It may be bispecific with bivalency for one target and monovalency for another. As illustrated in FIG. 14, the molecule is comprised of four VH domains with N—C-terminal glycine/serine rich linkers between each domain. This molecule may be configured to be tetraspecific, trispecific, or bispecific with varying antigenic valencies in each case. Again, in this format, any of the antibody moieties at can be substituted with a non-antibody moiety, e.g., a effector binding/modulating moiety that does not comprise an antibody molecule.

Other embodiments of bispecific antibodies are illustrated in FIGS. 15 and 16. FIGS. 15 and 16 are comprised of the naturally heterodimerizing core of the human IgG CH1/Ckappa interface, including the C-terminal heavy/light disulfide bond which covalently anchors the interaction. This format does not contain an Fc or any moieties for half-life extension. As illustrated in FIG. 15, the molecule, at the N-terminus of the Ckappa domain is appended with an scFv fragment consisting of an N-terminal VH domain, linked at its C-terminus to the N-terminus of a VL domain via a 12-15 amino acid glycine/serine based linker, which is linked by its C-terminus to the N-terminus of the Ckappa domain via the native VL-Ckappa elbow sequence. The CH1 domain is appended at the N-terminus with an scFv fragment consisting of an N-terminal VL domain linked at its C-terminus via a 12-15 amino acid glycine/serine linker to the N-terminus of a VH domain, which is linked at its C-terminus to the N-terminus of the CH1 domains via the natural VH-CH1 elbow sequence. As illustrated in FIG. 16, the molecule has the same N-terminal configuration to Example 13. However the C-terminus of the Ckappa and CH1 domains are appended with scFv modules which may be in either the VH-VL or VL-VH configuration and may be either specific for the same antigen or specific for two different antigens. The VH/VL inter-domain linkers may be 12-15 amino acids in length and consisting of glycine/serine residues. The scFv binding sub-units may be swapped for soluble VH domains, or peptide recognition elements, or even tandem-scFv elements. This approach can also be configured to use Vlambda and/or Clambda domains. Again, in this format, any of the antibody moieties at any of the attachment/fusion points can be substituted with a non-antibody moiety, e.g., a effector binding/modulating moiety that does not comprise an antibody molecule.

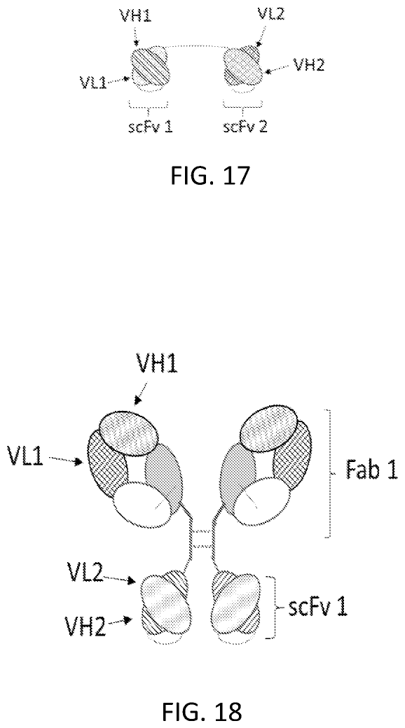

FIG. 17 illustrates another embodiment. FIG. 17 represents a tandem scFv format consisting of a first N-terminal VL domain linked at its C-terminus to the N-terminus of a first VH domain with a 12-15 amino acid glycine/serine rich linker, followed at the first VH C-terminus by a 25-30 amino acid glycine/serine/alanine/threonine based linker to the N-terminus of a second VL domain. The second VL domain is linked at the C-terminus to the N-terminus of a 2nd VH domain by a 12-15 amino acid glycine/serine linker. Each scFv recognizes a different target antigen such as a costimulatory T cell molecule and a tissue tethering target. Again, in this format, any of the antibody moieties can be substituted with a non-antibody moiety, e.g., a effector binding/modulating moiety that does not comprise an antibody molecule.

FIG. 18 illustrates another embodiment. FIG. 18 is a F(ab′)2 scFv fusion. This consists of two identical Fab components joined via two disulfide bonds in the native human IgG1 hinge region C-terminal of the human IgG CH1 domain. The human IgG1 CH2 and CH3 domains are absent. At the C-terminus of heavy chains 1 and 2 are two identical scFv fragments linked via a glycine/serine/alanine/threonine rich linker to the C-terminus of the huIgG1 hinge region. In the configuration shown, the VH is N-terminal in each scFv unit and linked via a 12-15 amino acid glycine/serine rich linker to the N-terminus of a VL domain. An alternative configuration would be N-term-VL-Linker-VH-C-term. In this design, the construct is bispecific with bivalency for reach target. Again, in this format, any of the antibody moieties at any of the four attachment/fusion points can be substituted with a non-antibody moiety, e.g., a effector binding/modulating moiety that does not comprise an antibody molecule.

As provided herein, the effector moiety that is linked or associated with can be a PD-1 agonist, IL-2 mutein, or a CD39 molecule. As used herein, the term “CD39 molecule” refers to a polypeptide having sufficient CD39 sequence that, as part of a therapeutic compound, phosphohydrolyzes ATP to AMP. In some embodiments, a CD39 molecule phosphohydrolizes ATP to AMP equivalent to, or at least, 10, 20, 30, 40, 50, 60, 70, 80, 90, or 95% of the rate of a naturally occurring CD39, e.g., the CD39 from which the CD39 molecule was derived. In some embodiments, a CD39 molecule has at least 60, 70, 80, 90, 95, 99, or 100% sequence identity, or substantial sequence identity, with a naturally occurring CD39. Any functional isoform can be used (with CD39 or other proteins discussed herein).

Exemplary CD39 sequence include Genbank accession #NP_001767.3 or a mature form from the following sequence:

(SEQ ID NO: 1)
MEDTKESNVKTFCSKNILAILGESSIIAVIALLAVGLTQNKALP
ENVKYGIVLDAGSSHTSLYIYKWPAEKENDTGVVHQVEECRVKG
PGISKFVQKVNEIGIYLTDCMERAREVIPRSQHQETPVYLGATA
GMRLLRMESEELADRVLDVVERSLSNYPFDFQGARIITGQEEGA
YGWITINYLLGKFSQKTRWFSIVPYETNNQETFGALDLGGASTQ
VTFVPQNQTIESPDNALQFRLYGKDYNVYTHSFLCYGKDQALWQ
KLAKDIQVASNEILRDPCFHPGYKKVVNVSDLYKTPCTKRFEMT
LPFQQFEIQGIGNYQQCHQSILELFNTSYCPYSQCAFNGIFLPP
LQGDFGAFSAFYFVMKFLNLTSEKVSQEKVTEMMKKFCAQPWEE
IKTSYAGVKEKYLSEYCFSGTYILSLLLQGYHFTADSWEHIHFI
GKIQGSDAGWTLGYMLNLINMIPAEQPLSTPLSHSTYVFLMVLF
SLVLFTVAIIGLLIFHKPSYFWKDMV.

In some embodiments, a CD39 molecule comprises a soluble catalytically active form of CD39 found to circulate in human or murine serum, see, e.g., Metabolism of circulating ADP in the bloodstream is mediated via integrated actions of soluble adenylate kinase-1 and NTPDase1/CD39 activities, Yegutkin et al. FASEB J. 2012 September; 26 (9): 3875-83. A soluble recombinant CD39 fragment is also described in Inhibition of platelet function by recombinant soluble ecto-ADPase/CD39, Gayle, et al., J Clin Invest. 1998 May 1; 101 (9): 1851-1859.

In some embodiments, the CD39 effector domain comprises a sequence of:

TQNKPLPENVKYGIVLDAGSSHTNLYIYKWPAEKENDTGVVQQLEECQVKGPGISKYAQKTDEIG
AYLAECMELSTELIPTSKHHQTPVYLGATAGMRLLRMESEQSADEVLAAVSTSLKGYPFDFQGAK
IITGQEEGAYGWITINYLLGRFTQEQSWLSLISDSQKQETFGALDLGGASTQITFVPQNSTIESP
ENSLQFRLYGEDYTVYTHSFLCYGKDQALWQKLAKDIQVSSGGVLKDPCFNPGYEKVVNVSELYG
TPCTERFEKKLPFDQFRIQGTGDYEQCHQSILELFNNSHCPYSQCAFNGVFLPPLHGSFGAFSAF
YFVMDFFKKVAKNSVISQEKMTEITKNFCSKSWEETKTSYPSVKEKYLSEYCFSGAYILSLLQGY
NFTDSSWEQIHFMGKIKDSNAGWTLGYMLNLTNMIPAEQPLSPPLPHSTYI (murine CD39,
SEQ ID NO: 470);
or
TQNKALPENVKYGIVLDAGSSHTSLYIYKWPAEKENDTGVVHQVEECRVKGPGISKFVQKVNEIG
IYLTDCMERAREVIPRSQHQETPVYLGATAGMRLLRMESEELADRVLDVVERSLSNYPFDFQGAR
IITGQEEGAYGWITINYLLGKFSQKTRWFSIVPYETNNQETFGALDLGGASTQVTFVPQNQTIES
PDNALQFRLYGKDYNVYTHSFLCYGKDQALWQKLAKDIQVASNEILRDPCFHPGYKKVVNVSDLY
KTPCTKRFEMTLPFQQFEIQGIGNYQQCHQSILELFNTSYCPYSQCAFNGIFLPPLQGDFGAFSA
FYFVMKFLNLTSEKVSQEKVTEMMKKFCAQPWEEIKTSYAGVKEKYLSEYCFSGTYILSLLLQGY
HFTADSWEHIHFIGKIQGSDAGWTLGYMLNLTNMIPAEQPLSTPLSHSTYV (human CD39,
SEQ ID NO: 471);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTLPFQQFEIQGIGNYQQCH
QSILELFNTSYCPYSQCAFNGIFLPPLQGDFGAFSAFYFVMKFLNLTSEKVSQEKVTEMMKKFCA
QPWEEIKTSYAGVKEKYLSEYCFSGTYILSLLLQGYHFTADSWEHIHFIGKIQGSDAGWTLGYML
NLTNMIPAEQPLSTPLSHSTYV (human CD39 (N'd8), SEQ ID NO: 472);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEAGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTLPFQQFEIQGIGNYQQCH
QSILELFNTSYCPYSQCAFNGIFLPPLQGDFGAFSAFYFVMKFLNLTSEKVSQEKVTEMMKKFCA
QPWEEIKTSYAGVKEKYLSEYCFSGTYILSLLLQGYHFTADSWEHIHFIGKIQGSDAGWTLGYML
NLTNMIPAEQPLSTPLSHSTYV (human CD39 (E174A), SEQ ID NO: 473);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRSEMTLPFQQFEIQGIGNYQQCH
QSILELENTSYCPYSQCAFNGIFLPPLQGDFGAFSAFYFVMKFLNLTSEKVSQEKVTEMMKKFCA
QPWEEIKTSYAGVKEKYLSEYCFSGTYILSLLLQGYHFTADSWEHIHFIGKIQGSDAGWTLGYML
NLTNMIPAEQPLSTPLSHSTYV (human CD39 (F305S), SEQ ID NO: 474);
or
NVKYGIVLDAGSSHTSLYIYKWPAEKENDTGVVHQVECRVKGPGISKEVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTEPFQQFEIQGIGNYQQCH
QSILELFNTSYCPYSQCAFNGIFLPPLQGDFGAFSAFYFVMKFLNLTSEKVSQEKVTEMMKKFCA
QPWEEIKTSYAGVKEKYLSEYCFSGTYILSLLLQGYHFTADSWEHIHFIGKIQGSDAGWTLGYML
NLTNMIPAEQPLSTPLSHSTYV (human CD39 (L309E), SEQ ID NO: 475);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTLPFQQTEIQGIGNYQQCH
QSILELFNTSYCPYSQCAFNGIFLPPLQGDFGAFSAFYFVMKFLNLTSEKVSQEKVTEMMKKFCA
QPWEEIKTSYAGVKEKYLSEYCFSGTYILSLLLQGYHFTADSWEHIHFIGKIQGSDAGWTLGYML
NLTNMIPAEQPLSTPLSHSTYV (human CD39 (F314T), SEQ ID NO: 476);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTLPFQQFEIQGIGNYQQCH
QSILELFNTSYCPYSQCAFNGIFLPPLQGDFGAFSAFYFVMKFLNLTSEKVSQEKVTEMMKKFCA
QPWEEIKTSYAGVKEKYLSEYCRSGTYILSLLLQGYHFTADSWEHIHFIGKIQGSDAGWTLGYML
NLTNMIPAEQPLSTPLSHSTYV (human CD39 (F414R), SEQ ID NO: 478);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTLPFQQFEIQGIGNYQQCH
QSILELENTSYCPYSQCAFNGIFLPPLQGDFGAFSAFYFVMKELNLTSEKVSQEKVTEMMKKFCA
QPWEEIKTSYAGVKEKYLSEYCFSGTYISSLLLQGYHFTADSWEHIHFIGKIQGSDAGWTLGYML
NLTNMIPAEQPLSTPLSHSTYV (human CD39 (L420S), SEQ ID NO: 479);
or
NVKYGIVLDAGSSHTSLYIYKWPAEKENDTGVVHQVECRVKGPGISKFVQKVNEIGIYLTDCMER
AREVIPRSQHQETPVYLGATAGMRLLRMESEELADRVLDVVERSLSNYPFDFQGARIITGQEEGA
YGWITINYLLGKFSQKTRWFSIVPYETNNQETFGALDLGGASTQVTFVPQNQTIHSFLCYGKDQA
LWQKLAKDIQVASNEILRDPCFHPGYKKVVNVSDLYKTPCTKRFEMTLPFQQFEIQGIGNYQQCH
QSILELFNTSYCPYSQCAFNGIFLPPLQGDFGAFSAFYFVMKFLNLTSEKVSQEKVTEMMKKFCA
QPWEEIKTSYAGVKEKYLSEYCFSGTYILSLLSQGYHFTADSWEHIHFIGKIQGSDAGWTLGYML
NLTNMIPAEQPLSTPLSHSTYV (human CD39 (L424S), SEQ ID NO: 480)

In some embodiments, the CD39 sequence comprises mutations. In some embodiments, the mutations are insertions, deletions, or substitutions as compared to SEQ ID NO: 470 or 471. In some embodiments, the CD39 sequence comprises F305S, L309E, F314T, F314T, F414R, L420S, or L424S mutations. CD39 can also be referred to as ENTPD1. Other members of this gene family can also be used as an effector molecule, such as ENTPD1, ENTPD2, ENTPD3, ENTPD4, ENTPD5, ENTPD6, ENTPD7, ENTPD8, or ENTPD9.

In some embodiments, the effector molecule is an ENTPD2 polypeptide, which comprises the sequence of:

(SEQ ID NO: 481)
TRDVREPPALKYGIVLDAGSSHTSMFIYKWPADKENDTGIVGQHSSCDVP
GGGISSYADNPSGASQSLVGCLEQALQDVPKERHAGTPLYLGATAGMRLL
NLTNPEASTSVLMAVTHTLTQYPFDERGARILSGQEEGVFGWVTANYLLE
NFIKYGWVGRWFRPRKGTLGAMDLGGASTQITFETTSPAEDRASEVQLHL
YGQHYRVYTHSFLCYGRDQVLQRLLASALQTHGFHPCWPRGFSTQVLLGD
VYQSPCTMAQRPQNFNSSARVSLSGSSDPHLCRDLVSGLFSFSSCPFSRC
SENGVFQPPVAGNFVAFSAFFYTVDFLRTSMGLPVATLQQLEAAAVNVCN
QTWAQLQARVPGQRARLADYCAGAMFVQQLLSRGYGFDERAFGGVIFQKK
AADTAVGWALGYMLNLTNLIPADPPGLRKGTDES

In some embodiments, the effector molecule is an ENTPD3 polypeptide, which comprises the sequence of:

(SEQ ID NO: 482)
QIHKQEVLPPGLKYGIVLDAGSSRTTVYVYQWPAEKENNTGVVSQTFKCS
VKGSGISSYGNNPQDVPRAFEECMQKVKGQVPSHLHGSTPIHLGATAGMR
LLRLONETAANEVLESIQSYFKSQPFDERGAQIISGQEEGVYGWITANYL
MGNFLEKNLWHMWVHPHGVETTGALDLGGASTQISFVAGEKMDLNTSDIM
QVSLYGYVYTLYTHSFQCYGRNEAEKKFLAMLLONSPTKNHLTNPCYPRD
YSISFTMGHVFDSLCTVDORPESYNPNDVITFEGTGDPSLCKEKVASIFD
FKACHDQETCSFDGVYQPKIKGPFVAFAGFYYTASALNLSGSFSLDTENS
STWNFCSQNWSQLPLLLPKFDEVYARSYCFSANYIYHLFVNGYKFTEETW
PQIHFEKEVGNSSIAWSLGYMLSLTNQIPAESPLIRLPIEPP

In some embodiments, the effector molecule is an ENTPD4 polypeptide, which comprises the sequence of:

(SEQ ID NO: 483)
KVEATSVLLPTDIKFGIVEDAGSSHTSLFLYQWLANKENGTGVVSQALAC
QVEGPGISSYTSNAAQAGESLQGCLEEALVLIPEAQHRKTPTFLGATAGM
RLLSRKNSSQARDIFAAVTQVLGRSPVDFWGAELLAGQAEGAFGWITVNY
GLGTLVKYSFTGEWIQPPEEMLVGALDMGGASTQITFVPGGPILDKSTQA
DFRLYGSDYSVYTHSYLCFGRDQMLSRLLVGLVQSRPAALLRHPCYLSGY
QTTLALGPLYESPCVHATPPLSLPQNLTVEGTGNPGACVSAIRELFNFSS
CQGQEDCAFDGVYQPPLRGQFYAFSNFYYTFHFLNLTSRQPLSTVNATIW
EFCQRPWKLVEASYPGQDRWLRDYCASGLYILTLLHEGYGFSEETWPSLE
FRKQAGGVDIGWTLGYMLNLTGMIPADAPAQWRAESYGVWVAK

The catalytic domains of these proteins can also be used in place of the sequences above. The catalytic domain of CD39 is referred to as the CD39 Effector Domain herein.

Elevated risk, as used herein, refers to the risk of a disorder in a subject, wherein the subject has one or more of (1) a medical history of the disorder or a symptom of the disorder, (2) a biomarker associated with the disorder or a symptom of the disorder, or (3) a family history of the disorder or a symptom of the disorder.

Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally occurring sequence, or are encoded by a substantially identical nucleotide sequence, and are capable of having one or more activities of the naturally occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some embodiments, the molecule comprises a CD39 molecule, an IL-2 mutein, or PD-1 agonist, such as an antibody.

Specific targeting moiety, as that term is used herein, refers to donor specific targeting moiety or a tissue specific targeting moiety.

Subject, as that term is used herein, refers to a mammalian subject, e.g., a human subject. In some embodiments, the subject is a non-human mammal, e.g., a horse, dog, cat, cow, goat, or pig.

Target ligand binding molecule, as used herein, refers to a polypeptide that has sufficient sequence from a naturally occurring counter ligand of a target ligand that it can bind the target ligand on a target tissue (e.g., donor tissue or subject target tissue) with sufficient specificity that it can serve as a specific targeting moiety. In some embodiments, a target ligand binding molecule binds to target tissue or cells with at least 10, 20, 30, 40, 50, 60, 70, 80, 90, or 95% of the affinity of the naturally occurring counter ligand. In some embodiments, a target ligand binding molecule has at least 60, 70, 80, 90, 95, 99, or 100% sequence identity, or substantial sequence identity, with a naturally occurring counter ligand for the target ligand.

Target site, as that term is used herein, refers to a site which contains the entity, e.g., epitope, bound by a targeting moiety. In some embodiments, the target site is the site at which immune privilege is established, such as the kidney or the structures within the kidney.

Tissue specific targeting moiety, as that term is used herein, refers to a moiety, e.g., an antibody molecule, that as a component of a therapeutic molecule, localizes the therapeutic molecule preferentially to a target tissue, as opposed to other tissue of a subject. As a component of a therapeutic compound, the tissue specific targeting moiety provides site-specific immune privilege for a target tissue, e.g., an organ or tissue undergoing or at risk for autoimmune attack. In some embodiments, a tissue specific targeting moiety binds to a product, e.g., a polypeptide product, which is not present outside the target tissue, or is present at sufficiently low levels that, at therapeutic concentrations of therapeutic molecule, unacceptable levels of immune suppression are absent or substantially absent. In some embodiments, a tissue specific targeting moiety binds to an epitope, which epitope is not present outside, or not substantially present outside, the target site.

In some embodiments, a tissue specific targeting moiety, as a component of a therapeutic compound, preferentially binds to a target tissue or target tissue antigen, e.g., has a binding affinity for the target tissue or antigen that is greater for target antigen or tissue, e.g., at least 2, 4, 5, 10, 50, 100, 500, 1,000, 5,000, or 10,000 fold greater, than its affinity for non-target tissue or antigen present outside the target tissue. Affinity of a therapeutic compound of which the tissue specific moiety is a component, can be measured in a cell suspension, e.g., the affinity for suspended cells having the target antigen is compared with its affinity for suspended cells not having the target antigen. In some embodiments, the binding affinity for the target antigen bearing cells is below 10 nM.

In some embodiments, the binding affinity for the target antigen bearing cells is below 100 pM, 50 pM, or 10 pM. In some embodiments, the specificity for a target antigen is sufficient, that when the tissue specific targeting moiety is coupled to an immune down regulating effector: i) immune attack of the target tissue, e.g., as measured by histological inflammatory response, infiltrating T effector cells, or organ function, in the clinical setting, e.g., creatinine for kidney, is substantially reduced, e.g., as compared to what would be seen in an otherwise similar implant but lacking the tissue specific targeting moiety is coupled to an immune down regulating effector; and/or ii) immune function in the recipient, outside or away from the target tissue, is substantially maintained.

In some embodiments, one or more of the following is seen: at therapeutic levels of therapeutic compound, peripheral blood lymphocyte counts are not substantially impacted, e.g., the level of T cells is within 25, 50, 75, 85, 90, or 95% of normal, the level of B cells is within 25, 50, 75, 85, 90, or 95% of normal, and/or the level of granulocytes (PMN cells) is within 25, 50, 75, 85, 90, or 95% of normal, or the level of monocytes is within 25, 50, 75, 85, 90, or 95% of normal; at therapeutic levels of therapeutic compound, the ex vivo proliferative function of PBMCs against non-disease relevant antigens is substantially normal or is within 70, 80, or 90% of normal; at therapeutic levels of therapeutic compound, the incidence or risk of opportunistic infections and cancers associated with immunosuppression is not substantially increased over normal; or at therapeutic levels of therapeutic compound, the incidence or risk of opportunistic infections and cancers associated with immunosuppression is substantially less than would be seen with standard of care, or non-targeted, immunosuppression. In some embodiments, the tissue specific targeting moiety comprises an antibody molecule. In some embodiments, the donor specific targeting moiety comprises an antibody molecule, a target specific binding polypeptide, or a target ligand binding molecule. In some embodiments, the tissue specific targeting moiety binds a product, or a site on a product, that is present or expressed exclusively, or substantially exclusively, on target tissue.

### Inhibitory Immune Checkpoint Molecules

In some embodiments, the effector domain is an “inhibitory immune checkpoint molecule ligand molecule,” as that term is used herein, refers to a polypeptide having sufficient inhibitory immune checkpoint molecule ligand sequence, e.g., in the case of a PD-L1 molecule, sufficient PD-L1 sequence, that when present as an ICIM binding/modulating moiety of a multimerized therapeutic compound, can bind and agonize its cognate inhibitory immune checkpoint molecule, e.g., again in the case of a PD-L1 molecule, PD-1.

In some embodiments, the inhibitory immune checkpoint molecule ligand molecule, e.g., a PD-L1 molecule, when binding as a monomer (or binding when the therapeutic compound is not multimerized), to its cognate ligand, e.g., PD-1, does not antagonize or substantially antagonize, or prevent binding, or prevent substantial binding, of an endogenous inhibitory immune checkpoint molecule ligand to the inhibitory immune checkpoint molecule. E.g., in the case of a PD-L1 molecule, the PD-L1 molecule does not antagonize binding of endogenous PD-L1 to PD-1.

In some embodiments, the inhibitory immune checkpoint molecule ligand when binding as a monomer, to its cognate inhibitory immune checkpoint molecule does not agonize or substantially agonize the inhibitory immune checkpoint molecule. By way of example, e.g., a PD-L1 molecule when binding to PD-1, does not agonize or substantially agonize PD-1.

In some embodiments, an inhibitory immune checkpoint molecule ligand molecule has at least 60, 70, 80, 90, 95, 99, or 100% sequence identity, or substantial sequence identity, with a naturally occurring inhibitory immune checkpoint molecule ligand.

Exemplary inhibitory immune checkpoint molecule ligand molecules include: a PD-L1 molecule, which binds to inhibitory immune checkpoint molecule PD-1, and in embodiments has at least 60, 70, 80, 90, 95, 99, or 100% sequence identity, or substantial sequence identity, with a naturally occurring PD-L1, e.g., the PD-L1 molecule comprising the sequence of MRIFAVFIFMTYWHLLNAFTVTVPKDLYVVEYGSNMTIECKFPVEKQLDLAALIVYWE MEDKNIIQFVHGEEDLKVQHSSYRQRARLLKDQLSLGNAALQITDVKLQDAGVYRCMI SYGGADYKRITVKVNAPYNKINQRILVVDPVTSEHELTCQAEGYPKAEVIWTSSDHQVL SGKTTTTNSKREEKLFNVTSTLRINTTTNEIFYCTFRRLDPEENHTAELVIPELPLAHPPNE RTHLVILGAILLCLGVALTFIFRLRKGRMMDVKKCGIQDTNSKKQSDTHLEET (SEQ ID NO: 3), or an active fragment thereof; in some embodiments, the active fragment comprises residues 19 to 290 of the PD-L1 sequence; a HLA-G molecule, which binds to any of inhibitory immune checkpoint molecules KIR2DL4, LILRB1, and LILRB2, and in embodiments has at least 60, 70, 80, 90, 95, 99, or 100% sequence identity, or substantial sequence identity, with a naturally occurring HLA-G. Exemplary HLA-G sequences include, e.g., a mature form found in the sequence at GenBank P17693.1 RecName: Full=HLA class I histocompatibility antigen, alpha chain G; AltName: Full=HLA G antigen; AltName: Full=MHC class I antigen G; Flags: Precursor, or in the sequence

(SEQ ID NO: 4)
MVVMAPRTLFLLLSGALTLTETWAGSHSMRYFSAAVSRPGRGEPRFIAMG
YVDDTQFVRFDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQ
SSLPTIPIMGIVA.

As provided for herein, the targeting moiety that binds to CDH16 or to OCT2 can be linked or associated with an inhibitory immune checkpoint molecule. Exemplary inhibitory molecules (e.g., an inhibitory immune checkpoint molecule) (together with their counter ligands) can be found in Table 1. This table lists molecules to which exemplary ICIM binding moieties can bind.

TABLE 1
Cell surface inhibitory molecules, e.g., inhibitory immune checkpoint molecules
(column A), counter ligands (column B) and cell types affected (column C).

| A | B | C |
|---|---|---|
| PD-1 | PD-L1, PD-L2 | T cells, B cells |
| Alkaline phosphatase | | |
| B7-H3 | Unknown | T cells |
| B7-H4 | Neuropilin 1, | T cells |
| | Neuropilin 2, | |
| | Plexin4A | |
| BTLA | HVEM | T cells, B cells |
| CTLA-4 | CD80, CD86 | T cells |
| IDO1 | Tryptophan | Lymphocytes |
| IDO2 | Tryptophan | Lymphocytes |
| KIR2DL1, | HLA MHC class I | NK cells |
| KIR2DL2/3, | | |
| KIR3DL1, KIR3DL2 | | |
| LAG3 | HLA MHC class II | T cells |
| TIM-3 | Galectin-9 | T cells |
| VISTA | Unknown | T cells, myeloid cells |
| TIGIT | CD155 | T cells |
| KIR2DL4 | HLA-G | NK cells |
| LILRB1/ILT2 | HLA-G/MHC Class | T cells, NK cells, B cells, monocytes, |
| | 1 | dendritic cells |
| LILRB2/ILT4 | HLA-G/MHC Class | Monocytes, dendritic cells, neutrophils, some |
| | 1 | tumor cells |
| LILRB3/ILT5 | Unknown | Myeloid cells |
| NKG2A | Nonclassical MHC | T cells, NK cells |
| | Glycoproteins class I | |
| FCRL1-6 | FCRL1 - 2 not | B cells |
| | known | |
| | FCRL4 = IgA | |
| | FCRL5 = IgG | |
| | FCRL6 = MHC Class | |
| | II | |
| | BUTYROPHILINS, | Modulation of immune cells |
| | for example | |
| | BTN1A1, BTN2A2, | |
| | BTNL2, BTNL1, | |
| | BTNL8 | |
| | CD39/ENTPD1 | Cell surface ecto-nucleoside triphosphate |
| | | diphosphohydrolase 1 converts pro- |
| | | inflammatory ATP to AMP |
| | CD73 | Cell surface ecto-5′-nucleotidase converts |
| | | AMP to anti-inflammatory Adenosine |
| | CD55/DAF | Indirectly blocks formation of complement |
| | | membrane attack complex by limiting |
| | | amplification of convertases |
| | CD59/MAC- | Directly inhibits formation of complement |
| | IP/MIRL | membrane attack complex |
| Siglec - 10 | CD24 | DC/B Cells/T Cells |
| SIRL1/VSTM1 | Not known | Neutrophils/myeloid cells |
| SIRP Alpha | CD47 | Monocytes/Macrophages |

In some embodiments, the anti-effector or inhibitory immune checkpoint molecule antibody molecule, when binding as a monomer (or binding when the therapeutic compound is not multimerized), to the effector or inhibitory immune checkpoint molecule, does not antagonize, substantially antagonize, prevent binding, or prevent substantial binding, of an endogenous counter ligand of the inhibitory immune checkpoint molecule to inhibitory immune checkpoint molecule. In some embodiments, the anti-effector or inhibitory immune checkpoint molecule antibody molecule when binding as a monomer (or binding when the therapeutic compound is not multimerized), to the inhibitory immune checkpoint molecule, does not agonize or substantially agonize, the effector or inhibitory molecule.

### The PD-L1/PD-1 Pathway

Programmed cell death protein 1, (often referred to as PD-1) is a cell surface receptor that belongs to the immunoglobulin superfamily. PD-1 is expressed on T cells and other cell types including, but not limited to, B cells, myeloid cells, dendritic cells, monocytes, T regulatory cells, iNK T cells. PD-1 binds two ligands, PD-L1 and PD-L2, and is an inhibitory immune checkpoint molecule. Engagement with a cognate ligand, PD-L1 or PD-L2, in the context of engagement of antigen loaded MHC with the T cell receptor on a T cell minimizes or prevents the activation and function of T cells. The inhibitory effect of PD-1 can include both promoting apoptosis (programmed cell death) in antigen specific T cells in lymph nodes and reducing apoptosis in regulatory T cells (suppressor T cells).

In some embodiments, a therapeutic compound, such as the polypeptides comprising a targeting moiety that binds to CDH16, comprises an ICIM binding/modulating moiety which agonizes PD-1 inhibition. In some embodiments, a therapeutic compound, such as the polypeptides comprising a targeting moiety that binds to OCT2, comprises an ICIM binding/modulating moiety which agonizes PD-1 inhibition. An ICIM binding/modulating moiety can include an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1.

In some embodiments, a therapeutic compound, such as the polypeptides provided herein, does not bind, or does not substantially bind, other tissues. In some embodiments, the therapeutic compound, such as the polypeptides provided herein, specifically binds to the kidney tissue. In some embodiments, the therapeutic compound comprises an ICIM binding/modulating moiety, e.g., an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1, such that the therapeutic compound, e.g., when bound to target, activates PD-1. In some embodiments, the therapeutic compound targets an allograft and provides local immune privilege to the allograft.

IL-2 Mutein Molecules: IL-2 Receptor Binders that Activate Tregs

IL-2 mutein molecule, as that term is used herein, refers to an IL-2 variant that binds with high affinity to the CD25 (IL-2R alpha chain) and with low affinity to the other IL-2R signaling components CD122 (IL-2R beta) and CD132 (IL-2R gamma). Such an IL-2 mutein molecule preferentially activates Treg cells. In embodiments, either alone, or as a component of a therapeutic compound, an IL-2 mutein activates Tregs at least 2, 5, 10, or 100 fold more than cytotoxic or effector T cells. Exemplary IL-2 mutein molecules are described in WO2010085495, WO2016/164937, US2014/0286898A1, WO2014153111A2, WO2010/085495, cytotoxic WO2016014428A2, WO2016025385A1, and US20060269515. Muteins disclosed in these references that include additional domains, e.g., an Fc domain, or other domain for extension of half-life can be used in the therapeutic compounds and methods described herein without such additional domains. In another embodiment an IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof, e.g., AMG 592. In an embodiment the therapeutic compound comprises the IL-2 portion of AMG 592. In an embodiment the therapeutic compound comprises the IL-2 portion but not the immunoglobulin portion of AMG 592. In some embodiments, the mutein does not comprise a Fc region. For some IL-2 muteins, the muteins are engineered to contain a Fc region because such region has been shown to increase the half-life of the mutein. In some embodiments, the extended half-life is not necessary for the methods described and embodied herein. In some embodiments, the Fc region that is fused with the IL-2 mutein comprises a N297 mutations, such as, but not limited to, N297A. In some embodiments, the Fc region that is fused with the IL-2 mutein does not comprise a N297 mutation, such as, but not limited to, N297A.

IL-2 mutein molecules that preferentially expand or stimulate Treg cells (over cytotoxic T cells) can be used as an IIC binding/modulating moiety.

In some embodiments, IIC binding/modulating moiety comprises a IL-2 mutein molecule. As used herein, the term “IL-2 mutein molecule” or “IL-2 mutein” refers to an IL-2 variant that preferentially activates Treg cells. In some embodiments, either alone, or as a component of a therapeutic compound, an IL-2 mutein molecule activates Tregs at least 2, 5, 10, or 100 fold more than cytotoxic T cells. A suitable assay for evaluating preferential activation of Treg cells can be found in U.S. Pat. No. 9,580,486 at, for example, Examples 2 and 3, or in WO2016014428 at, for example, Examples 3, 4, and 5, each of which is incorporated by reference in its entirety. The sequence of mature IL-2 is

(mature IL-2 sequence)
(SEQ ID NO: 6)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

The immature sequence of IL-2 can be represented by

(SEQ ID NO: 15)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT.

In some embodiments, an IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof.

An IL-2 mutein molecule can be prepared by mutating one or more of the residues of IL-2. Non-limiting examples of IL-2-muteins can be found in WO2016/164937, U.S. Pat. Nos. 9,580,486, 7,105,653, 9,616,105, 9,428,567, US2017/0051029, US2014/0286898A1, WO2014153111A2, WO2010/085495, WO2016014428A2, WO2016025385A1, and US20060269515, each of which are incorporated by reference in its entirety.

In some embodiments, the alanine at position 1 of the sequence above is deleted. In some embodiments, the IL-2 mutein molecule comprises a serine substituted for cysteine at position 125 of the mature IL-2 sequence. Other combinations of mutations and substitutions that are IL-2 mutein molecules are described in US20060269515, which is incorporated by reference in its entirety. In some embodiments, the cysteine at position 125 is also substituted with a valine or alanine. In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, R81A, R81G, R81S, R81T, D84A, D84E, D84G, D84I, D84M, D84Q D84R, D84S, D84T, S87R, N88A, N88D, N88E, N88I, N88F, N88G, N88M, N88R, N88S, N88V, N88W, V91D, V91E, V91G, V91S, 192K, 192R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81S, R81T, D84A, D84E, D84G, D84I, D84M, D84Q, D84R, D84S, D84T, S87R, N88A, N88D, N88E, N88F, N88I, N88G, N88M, N88R, N88S, N88V, N88W, V91D, V91E, V91G, V91S, 192K, 192R, E95G, Q126I, Q126L, and Q126F. In some embodiments, the IL-2 mutein molecule differs from the amino acid sequence set forth in mature IL-2 sequence with a C125A or C125S substitution and with one substitution selected from D20H, D201, D20Y, D20E, D20G, D20W, D84A, D84S, H16D, H16G, H16K, H16R, H16T, H16V, 192K, 192R, L12K, L19D, L19N, L19T, N88D, N88R, N88S, V91D, V91G, V91K, and V91S. In some embodiments, the IL-2 mutein comprises N88R and/or D20H mutations.

In some embodiments, the IL-2 mutein molecule comprises a mutation in the polypeptide sequence at a position selected from the group consisting of amino acid 30, amino acid 31, amino acid 35, amino acid 69, and amino acid 74. In some embodiments, the mutation at position 30 is N30S. In some embodiments, the mutation at position 31 is Y31H. In some embodiments, the mutation at position 35 is K35R. In some embodiments, the mutation at position 69 is V69A. In some embodiments, the mutation at position 74 is Q74P. In some embodiments, the mutein comprises a V69A mutation, a Q74P mutation, a N88D or N88R mutation, and one or more of L53I, L56I, L80I, or L118I mutations. In some embodiments, the mutein comprises a V69A mutation, a Q74P mutation, a N88D or N88R mutation, and a L to I mutation selected from the group consisting of: L53I, L56I, L80I, and L118I mutation. In some embodiments, the IL-2 mutein comprises a V69A, a Q74P, a N88D or N88R mutation, and a L53I mutation. In some embodiments, the IL-2 mutein comprises a V69A, a Q74P, a N88D or N88R mutation, and a L56I mutation. In some embodiments, the IL-2 mutein comprises a V69A, a Q74P, a N88D or N88R mutation, and a L80I mutation. In some embodiments, the IL-2 mutein comprises a V69A, a Q74P, a N88D or N88R mutation, and a L118I mutation. As provided for herein, the muteins can also comprise a C125A or C125S mutation.

In some embodiments, the IL-2 mutein molecule comprises a substitution selected from the group consisting of: N88R, N88I, N88G, D20H, D109C, Q126L, Q126F, D84G, or D84I relative to mature human IL-2 sequence provided above. In some embodiments, the IL-2 mutein molecule comprises a substitution of D109C and one or both of a N88R substitution and a C125S substitution. In some embodiments, the cysteine that is in the IL-2 mutein molecule at position 109 is linked to a polyethylene glycol moiety, wherein the polyethylene glycol moiety has a molecular weight of between 5 and 40 kDa.

In some embodiments, any of the substitutions described herein are combined with a substitution at position 125. The substitution can be a C125S, C125A, or C125V substitution.

In addition to the substitutions or mutations described herein, in some embodiments, the IL-2 mutein has a substitution/mutation at one or more of positions 73, 76, 100, or 138 that correspond to SEQ ID NO: 15 or positions at one or more of positions 53, 56, 80, or 118 that correspond to SEQ ID NO: 6. In some embodiments, the IL-2 mutein comprises a mutation at positions 73 and 76; 73 and 100; 73 and 138; 76 and 100; 76 and 138; 100 and 138; 73, 76, and 100; 73, 76, and 138; 73, 100, and 138; 76, 100 and 138; or at each of 73, 76, 100, and 138 that correspond to SEQ ID NO: 15. In some embodiments, the IL-2 mutein comprises a mutation at positions 53 and 56; 53 and 80; 53 and 118; 56 and 80; 56 and 118; 80 and 118; 53, 56, and 80; 53, 56, and 118; 53, 80, and 118; 56, 80 and 118; or at each of 53, 56, 80, and 118 that correspond to SEQ ID NO: 6. As the IL-2 can be fused or tethered to other proteins, as used herein, the term corresponds to as reference to a SEQ ID NOs: 6 or 15 refer to how the sequences would align with default settings for alignment software, such as can be used with the NCBI website. In some embodiments, the mutation is leucine to isoleucine. Thus, the IL-2 mutein can comprise one more isoleucines at positions 73, 76, 100, or 138 that correspond to SEQ ID NO: 15 or positions at one or more of positions 53, 56, 80, or 118 that correspond to SEQ ID NO: 6. In some embodiments, the mutein comprises a mutation at L53 that correspond to SEQ ID NO: 6. In some embodiments, the mutein comprises a mutation at L56 that correspond to SEQ ID NO: 6. In some embodiments, the mutein comprises a mutation at L80 that correspond to SEQ ID NO: 6. In some embodiments, the mutein comprises a mutation at L118 that correspond to SEQ ID NO: 6. In some embodiments, the mutation is leucine to isoleucine. In some embodiments, the mutein also comprises a mutation as position 69, 74, 88, 125, or any combination thereof in these muteins that correspond to SEQ ID NO: 6. In some embodiments, the mutation is a V69A mutation. In some embodiments, the mutation is a Q74P mutation. In some embodiments, the mutation is a N88D or N88R mutation. In some embodiments, the mutation is a C125A or C125S mutation.

In some embodiments, the IL-2 mutein comprises a mutation at one or more of positions 49, 51, 55, 57, 68, 89, 91, 94, 108, and 145 that correspond to SEQ ID NO: 15 or one or more positions 29, 31, 35, 37, 48, 69, 71, 74, 88, and 125 that correspond to SEQ ID NO: 6. The substitutions can be used alone or in combination with one another. In some embodiments, the IL-2 mutein comprises substitutions at 2, 3, 4, 5, 6, 7, 8, 9, or each of positions 49, 51, 55, 57, 68, 89, 91, 94, 108, and 145. Non-limiting examples such combinations include, but are not limited to, a mutation at positions 49, 51, 55, 57, 68, 89, 91, 94, 108, and 145; 49, 51, 55, 57, 68, 89, 91, 94, and 108; 49, 51, 55, 57, 68, 89, 91, and 94; 49, 51, 55, 57, 68, 89, and 91; 49, 51, 55, 57, 68, and 89; 49, 51, 55, 57, and 68; 49, 51, 55, and 57; 49, 51, and 55; 49 and 51; 51, 55, 57, 68, 89, 91, 94, 108, and 145; 51, 55, 57, 68, 89, 91, 94, and 108; 51, 55, 57, 68, 89, 91, and 94; 51, 55, 57, 68, 89, and 91; 51, 55, 57, 68, and 89; 55, 57, and 68; 55 and 57; 55, 57, 68, 89, 91, 94, 108, and 145; 55, 57, 68, 89, 91, 94, and 108; 55, 57, 68, 89, 91, and 94; 55, 57, 68, 89, 91, and 94; 55, 57, 68, 89, and 91; 55, 57, 68, and 89; 55, 57, and 68; 55 and 57; 57, 68, 89, 91, 94, 108, and 145; 57, 68, 89, 91, 94, and 108; 57, 68, 89, 91, and 94; 57, 68, 89, and 91; 57, 68, and 89; 57 and 68; 68, 89, 91, 94, 108, and 145; 68, 89, 91, 94, and 108; 68, 89, 91, and 94; 68, 89, and 91; 68 and 89; 89, 91, 94, 108, and 145; 89, 91, 94, and 108; 89, 91, and 94; 89 and 91; 91, 94, 108, and 145; 91, 94, and 108; 91, and 94; or 94 and 108. Each mutation can be combined with one another. The same substitutions can be made in SEQ ID NO: 6, but the numbering would adjusted appropriately as is clear from the present disclosure (20 less than the numbering for SEQ ID NO: 15 corresponds to the positions in SEQ ID NO: 6).

In some embodiments, the IL-2 mutein comprises a mutation at one or more positions of 35, 36, 42, 104, 115, or 146 that correspond to SEQ ID NO: 15 or the equivalent positions at SEQ ID NO: 6 (e.g., positions 15, 16, 22, 84, 95, or 126). These mutations can be combined with the other leucine to isoleucine mutations described herein or the mutation at positions 73, 76, 100, or 138 that correspond to SEQ ID NO: 15 or at one or more of positions 53, 56, 80, or 118 that correspond to SEQ ID NO: 6. In some embodiments, the mutation is a E35Q, H36N, Q42E, D104N, E115Q, or Q146E, or any combination thereof. In some embodiments, one or more of these substitutions is wild-type. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g., positions 15, 16, 22, 84, 95, and 126).

The mutations at these positions can be combined with any of the other mutations described herein, including, but not limited to substitutions at positions 73, 76, 100, or 138 that correspond to SEQ ID NO: 15 or positions at one or more of positions 53, 56, 80, or 118 that correspond to SEQ ID NO: 6 described herein and above. In some embodiments, the IL-2 mutein comprises a N49S mutation that corresponds to SEQ ID NO: 15. In some embodiments, the IL-2 mutein comprises a Y51S or a Y51H mutation that corresponds to SEQ ID NO: 15. In some embodiments, the IL-2 mutein comprises a K55R mutation that corresponds to SEQ ID NO: 15. In some embodiments, the IL-2 mutein comprises a T57A mutation that corresponds to SEQ ID NO: 15. In some embodiments, the IL-2 mutein comprises a K68E mutation that corresponds to SEQ ID NO: 15. In some embodiments, the IL-2 mutein comprises a V89A mutation that corresponds to SEQ ID NO: 15. In some embodiments, the IL-2 mutein comprises a N91R mutation that corresponds to SEQ ID NO: 15. In some embodiments, the IL-2 mutein comprises a Q94P mutation that corresponds to SEQ ID NO: 15. In some embodiments, the IL-2 mutein comprises a N108D or a N108R mutation that corresponds to SEQ ID NO: 15. In some embodiments, the IL-2 mutein comprises a C145A or C145S mutation that corresponds to SEQ ID NO: 15. These substitutions can be used alone or in combination with one another. In some embodiments, the mutein comprises each of these substitutions. In some embodiments, the mutein comprises 1, 2, 3, 4, 5, 6, 7, or 8 of these mutations. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g. positions 15, 16, 22, 84, 95, and 126).

In some embodiments, the IL-2 mutein comprises a N29S mutation that corresponds to SEQ ID NO: 6. In some embodiments, the IL-2 mutein comprises a Y31S or a Y31H mutation that corresponds to SEQ ID NO: 6. In some embodiments, the IL-2 mutein comprises a K35R mutation that corresponds to SEQ ID NO: 6. In some embodiments, the IL-2 mutein comprises a T37A mutation that corresponds to SEQ ID NO: 6. In some embodiments, the IL-2 mutein comprises a K48E mutation that corresponds to SEQ ID NO: 6. In some embodiments, the IL-2 mutein comprises a V69A mutation that corresponds to SEQ ID NO: 6. In some embodiments, the IL-2 mutein comprises a N71R mutation that corresponds to SEQ ID NO: 6. In some embodiments, the IL-2 mutein comprises a Q74P mutation that corresponds to SEQ ID NO: 6. In some embodiments, the IL-2 mutein comprises a N88D or a N88R mutation that corresponds to SEQ ID NO: 6. In some embodiments, the IL-2 mutein comprises a C125A or C125S mutation that corresponds to SEQ ID NO: 6. These substitutions can be used alone or in combination with one another. In some embodiments, the mutein comprises 1, 2, 3, 4, 5, 6, 7, or 8 of these mutations. In some embodiments, the mutein comprises each of these substitutions. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g., positions 15, 16, 22, 84, 95, and 126).

For any of the IL-2 muteins described herein, in some embodiments, one or more of positions 35, 36, 42, 104, 115, or 146 that correspond to SEQ ID NO: 15 or the equivalent positions at SEQ ID NO: 6 (e.g., positions 15, 16, 22, 84, 95, or 126) are wild-type (e.g., are as shown in SEQ ID NOs: 6 or 15). In some embodiments, 2, 3, 4, 5, 6, or each of positions 35, 36, 42, 104, 115, or 146 that correspond to SEQ ID NO: 15 or the equivalent positions at SEQ ID NO: 6 (e.g., positions 15, 16, 22, 84, 95, and 126) are wild-type.

In some embodiments, the IL-2 mutein comprises a sequence of:

(SEQ ID NO: 16)
MYRMQLLSCIALSLALVTNSAPTSSSTKKTQLQLEHLLLDLQMILNGISN
HKNPRLARMLTFKFYMPEKATEIKHLQCLEEELKPLEEALRLAPSKNFHL
RPRDLISDINVIVLELKGSETTFMCEYADETATIVEFLNRWITFSQSIIS
TLT

In some embodiments, the IL-2 mutein comprises a sequence of:

(SEQ ID NO: 17)
MYRMQLLSCIALSLALVTNSAPTSSSTKKTQLQLEHLLLDLQMILNGISN
HKNPRLARMLTFKFYMPEKATELKHIQCLEEELKPLEEALRLAPSKNFHL
RPRDLISDINVIVLELKGSETTFMCEYADETATIVEFLNRWITFSQSIIS
TLT

In some embodiments, the IL-2 mutein comprises a sequence of:

(SEQ ID NO: 18)
MYRMQLLSCIALSLALVTNSAPTSSSTKKTQLQLEHLLLDLQMILNGISN
HKNPRLARMLTFKFYMPEKATELKHLQCLEEELKPLEEALRLAPSKNFHI
RPRDLISDINVIVLELKGSETTFMCEYADETATIVEFLNRWITFSQSIIS
TLT

In some embodiments, the IL-2 mutein comprises a sequence of:

(SEQ ID NO: 19)
MYRMQLLSCIALSLALVTNSAPTSSSTKKTQLQLEHLLLDLQMILNGISN
HKNPRLARMLTFKFYMPEKATELKHLQCLEEELKPLEEALRLAPSKNFHL
RPRDLISDINVIVLELKGSETTFMCEYADETATIVEFINRWITFSQSIIS
TLT

In some embodiments, the IL-2 mutein sequences described herein do not comprise the IL-2 leader sequence. The IL-2 leader sequence can be represented by the sequence of MYRMQLLSCIALSLALVTNS (SEQ ID NO: 20). Therefore, in some embodiments, the sequences illustrated above can also encompass peptides without the leader sequence. Although SEQ ID NOs; 16-20 are illustrated with only mutation at one of positions 73, 76, 100, or 138 that correspond to SEQ ID NO: 15 or positions at one or more of positions 53, 56, 80, or 118 that correspond to SEQ ID NO: 6, the peptides can comprises one, two, three or 4 of the mutations at these positions. In some embodiments, the substitution at each position is isoleucine or other type of conservative amino acid substitution. In some embodiments, the leucine at the recited positions are substituted with, independently, isoleucine, valine, methionine, or phenylalanine.

In some embodiments, the IL-2 mutein molecule is fused to a Fc Region or other linker region as described herein. Examples of such fusion proteins can be found in U.S. Pat. Nos. 9,580,486, 7,105,653, 9,616,105, 9,428,567, US2017/0051029, WO2016/164937, US2014/0286898A1, WO2014153111A2, WO2010/085495, WO2016014428A2, WO2016025385A1, US2017/0037102, and US2006/0269515, each of which are incorporated by reference in its entirety.

In some embodiments, the Fc region comprises what is known as the LALA mutation. Using the Kabat numbering of the Fc region, this would correspond to L247A, L248A, and G250A. In some embodiments, using the EU numbering of the Fc region, the Fc region comprises a L234A mutation, a L235A mutation, and/or a G237A mutation. Regardless of the numbering system used, in some embodiments, the Fc portion can comprise mutations that correspond to these residues. In some embodiments, the Fc region comprises N297G or N297A (Kabat numbering) mutations. The Kabat numbering is based upon a full-length sequence, but would be used in a fragment based upon a traditional alignment used by one of skill in the art for the Fc region.

In some embodiments, the Fc region comprises a sequence of:

(SEQ ID NO: 21)
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPG.
or
(SEQ ID NO: 28)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPG.

In some embodiments, the IL-2 mutein is linked to the Fc region. Non-limiting examples of linkers are glycine/serine linkers. For example, a glycine/serine linkers can be a sequence of GGGGSGGGGGGGGSGGGGS (SEQ ID NO: 22), GGGGSGGGGS (SEQ ID NO: 484), or GGGGSGGGGSGGGGS (SEQ ID NO: 30). This is simply a non-limiting example and the linker can have varying number of GGGGS (SEQ ID NO: 23) or GGGGA repeats (SEQ ID NO: 29). In some embodiments, the linker comprises 1, 2, 3, 4, 5, 6, 7, 8, 9, or 10 of the GGGGS (SEQ ID NO: 23) or GGGGA repeats (SEQ ID NO: 29) repeats.

Thus, the IL-2/Fc fusion can be represented by the formula of Z_{IL-2M}-L_{gs}-Z_{Fc}, wherein Z_{IL-2M} is a IL-2 mutein as described herein, L_{gs} is a linker sequence as described herein (e.g., glycine/serine linker) and Z_{Fc} is a Fc region described herein or known to one of skill in the art. In some embodiments, the formula can be in the reverse orientation Z_{Fc}-L_{gs}-Z_{IL-2M}.

In some embodiments, the IL-2/Fc fusion comprises a sequence of

(SEQ ID NO: 24)
MYRMQLLSCIALSLALVTNSAPTSSSTKKTQLQLEHLLLDLQMILNGISN
HKNPRLARMLTFKFYMPEKATEIKHLQCLEEELKPLEEALRLAPSKNFHL
RPRDLISDINVIVLELKGSETTFMCEYADETATIVEFLNRWITFSQSIIS
TLTGGGGSGGGGSGGGGSGGGGSDKTHTCPPCPAPEAAGAPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(SEQ ID NO: 25)
MYRMQLLSCIALSLALVTNSAPTSSSTKKTQLQLEHLLLDLQMILNGISN
HKNPRLARMLTFKFYMPEKATELKHIQCLEEELKPLEEALRLAPSKNFHL
RPRDLISDINVIVLELKGSETTFMCEYADETATIVEFLNRWITFSQSIIS
TLTGGGGSGGGGSGGGGSGGGGSDKTHTCPPCPAPEAAGAPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(SEQ ID NO: 26)
MYRMQLLSCIALSLALVTNSAPTSSSTKKTQLQLEHLLLDLQMILNGISN
HKNPRLARMLTFKFYMPEKATELKHLQCLEEELKPLEEALRLAPSKNFHI
RPRDLISDINVIVLELKGSETTFMCEYADETATIVEFLNRWITFSQSIIS
TLTGGGGSGGGGSGGGGSGGGGSDKTHTCPPCPAPEAAGAPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(SEQ ID NO: 27)
MYRMQLLSCIALSLALVTNSAPTSSSTKKTQLQLEHLLLDLQMILNGISN
HKNPRLARMLTFKFYMPEKATELKHLQCLEEELKPLEEALRLAPSKNFHL
RPRDLISDINVIVLELKGSETTFMCEYADETATIVEFINRWITFSQSIIS
TLTGGGGSGGGGSGGGGSGGGGSDKTHTCPPCPAPEAAGAPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG.

In some embodiments, the IL-2/Fc fusion comprises a sequence selected from the following table, Table 2

TABLE 2
IL-2/Fc Fusion Protein Amino Acid Sequences

| Sequence | |
|---|---|
| Identification | Sequence |
| SEQ ID NO: 7 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGAGGGGDKTHTCPPCPAPELLGGPSVFLFPPKPKDTLMISRTPEVTC |
| | VVVDVSHEDPEVKFNWYVDGVEVHNAKTKPREEQYASTYRVVSVLTVLHQDWLNGKEYKCK |
| | VSNKALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQKSLSLS |
| | PGK |
| SEQ ID NO: 8 | APTSSSTKKTQLQLEHLLLHLQMILNGINNYKNPKLTRMLTFKFYMPKKATELKHLQCLEE |
| | ELKPLEEVLNLAQSKNFHLRPRDLISNINVIVLELKGSETTFMCEYADETATIVEFLNRWI |
| | TFSQSIISTLTVECPPCPAPPVAGPSVFLFPPKPKDTLMISRTPEVTCVVVDVSHEDPEVQ |
| | FNWYVDGVEVHNAKTKPREEQFNSTFRVVSVLTVVHQDWLNGKEYKCKVSNKGLPAPIEKT |
| | ISKTKGQPREPQVYTLPPSREEMTKNQVSLTCLVKGFYPSDIAVEWESNGQPENNYKTTPP |
| | MLDSDGSFFLYSKLTVDKSRWQQGNVFSCSVMHEALHNHYTQKSLSLSPGK |
| SEQ ID NO: 9 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPG |
| SEQ ID NO: 10 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKCKVSNK |
| | ALPAPIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLSLSPG |
| SEQ ID NO: 11 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSDKTHTCPPCPAPELLGGPSVFLFPPKPKDTLMISRTPEVT |
| | CVVVDVSHEDPEVKFNWYVDGVEVHNAKTKPREEQYASTYRVVSVLTVLHQDWLNGKEYKC |
| | KVSNKALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYTQKSLSL |
| | SPG |
| SEQ ID NO: 12 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSGGGGSDKTHTCPPCPAPELLGGPSVFLFPPKPKDTLMISR |
| | TPEVTCVVVDVSHEDPEVKFNWYVDGVEVHNAKTKPREEQYASTYRVVSVLTVLHQDWLNG |
| | KEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNYHTQ |
| | KSLSLSPG |
| SEQ ID NO: 13 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSGGGGSGGGGSDKTHTCPPCPAPELLGGPSVFLFPPKPKDT |
| | LMISRTPEVTCVVVDVSHEDPEVKENWYVDGVEVHNAKTKPREEQYASTYRVVSVLTVLHQ |
| | DWLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEALH |
| | NHYTQKSLSLSPG |
| SEQ ID NO: 14 | APTSSSTKKTQLQLEHLLLHLQMILNGINNYKNPKLTRMLTFKFYMPKKATELKHLQCLEE |
| | ELKPLEEVLNLAQSKNFHLRPRDLISNINVIVLELKGSETTFMCEYADETATIVEFLNRWI |
| | TFSQSIISTLTGGGGSGGGGSGGGGSDKTHTCPPCPAPELLGGPSVFLFPPKPKDTLMISR |
| | TPEVTCVVVDVSHEDPEVKFNWYVDGVEVHNAKTKPREEQYASTYPVVSVLTVLHQDWING |
| | KEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQ |
| | KSLSLSPG |

In some embodiments, the IL-2 muteins comprises one or more of the sequences provided in the following table, which, in some embodiments, shows the IL-2 mutein fused with other proteins or linkers. The table also provides sequences for a variety of Fc domains or variants that the IL-2 can be fused with:

| SEQ ID | Brief | |
|---|---|---|
| NO: | Description | Amino Acid Sequence |
| 31 | Human IL-2 | APTSSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | mutation | TTFMCEYADETATIVEFLNRWITFSQSIISTLT |
| 32 | Human IL-2 | APASSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | and T3A | TTFMCEYADETATIVEFLNRWITFSQSIISTLT |
| | mutations | |
| 33 | Human IL-2 | APTSSSTKKTQLQLEHLLLDLQMILNGINNYKNPKLTRMLTFKFYMPKKA |
| | with N88R and | TELKHLQCLEEELKPLEEVLNLAQSKNFHLRPRDLISRINVIVLELKGSE |
| | C125S | TTFMCEYADETATIVEFLNRWITFSQSIISTLT |
| 34 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISNINVIVLELKGSE |
| | Q74P and | TTFMCEYADETATIVEFLNRWITFSQSIISTLT |
| | C125S | |
| | mutations | |
| 35 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D | TTFMCEYADETATIVEFLNRWITFSQSIISTLT |
| | and C125S | |
| | mutations | |
| 36 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISRINVIVLELKGSE |
| | Q74P, N88R | TTFMCEYADETATIVEFLNRWITFSQSIISTLT |
| | and C125S | |
| | mutations | |
| 37 | Human IL-2 | APTSSSTKKTQLQLEHLLLDLQMILNGINNYKNPKLTRMLTFKFYMPKKA |
| | with N88D and | TELKHLQCLEEELKPLEEVLNLAQSKNFHLRPRDLISDINVIVLELKGSE |
| | C125S | TTFMCEYADETATIVEFLNRWITFSQSIISTLT |
| 38 | Human IL-2 | APTSSSTKKTQLQLEHLLLDLQMILNGINNYKNPKLTRMLTFKFYMPKKA |
| | with L53I, | TEIKHLQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 39 | Human IL-2 | APTSSSTKKTQLQLEHLLLDLQMILNGINNYKNPKLTRMLTFKFYMPKKA |
| | with L56I, | TELKHIQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 40 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHIRPRDLISDINVIVLELKGSE |
| | Q74P, L80I, | TTFMCEYADETATIVEFLNRWITFSQSIISTLT |
| | N88D and | |
| | C125S | |
| | mutations | |
| 41 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D, | TTFMCEYADx`ETATIVEFINRWITFSQSIISTLT |
| | L118I, and | |
| | C125S | |
| | mutations | |
| 42 | Human IgG1 Fc | DKTHTCPPCPAPEAAGAPSVFLFPPKPKDTLMISRTPEVTCVVVDVSHED |
| | (N-terminal | PEVKFNWYVDGVEVHNAKTKPREEQYNSTYRVVSVLTVLHQDWLNGKEYK |
| | fusions) with | CKVSNKALPAPIEKTISKAKGQPREPQVYTLPPSREEMTKNQVSLTCLVK |
| | L234A, L235A, | GFYPSDIAVEWESNGQPENNYKTTPPVLDSDGSFFLYSKLTVDKSRWQQG |
| | and G237A | NVFSCSVMHEALHNHYTQKSLSLSPG |
| | mutations | |
| 30 | GGGGSGGGGSGGG | GGGGSGGGGSGGGGS |
| | GS linker (15 | |
| | amino acids) | |
| 22 | GGGGSGGGGSGGG | GGGGSGGGGSGGGGSGGGGS |
| | GSGGGGS | |
| | linker (20 | |
| | amino acids) | |
| 23 | GGGGS linker | GGGGS |
| | (5 amino | |
| | acids) | |
| 43 | Human IgG1 Fc | DKTHTCPPCPAPELLGGPSVFLFPPKPKDTLMISRTPEVTCVVVDVSHED |
| | (truncated) | PEVKFNWYVDGVEVHNAKTKPREEQYGSTYRVVSVLTVLHQDWLNGKEYK |
| | with N297G | CKVSNKALPAPIEKTISKAKGQPREPQVYTLPPSREEMTKNQVSLTCLVK |
| | mutation | GFYPSDIAVEWESNGQPENNYKTTPPVLDSDGSFFLYSKLTVDKSRWQQG |
| | | NVFSCSVMHEALHNHYTQKSLSLSPG |
| 44 | Antibody | ASTKGPSVFPLAPSSKSTSGGTAALGCLVKDYFPEPVTVSWNSGALTSGV |
| | Heavy Chain | HTFPAVLQSSGLYSLSSVVTVPSSSLGTQTYICNVNHKPSNTKVDKKVEP |
| | CH1-CH2-CH3 | KSCDKTHTCPPCPAPEAAGAPSVELFPPKPKDTLMISRTPEVTCVVVDVS |
| | domains | HEDPEVKFNWYVDGVEVHNAKTKPREEQYNSTYRVVSVLTVLHQDWLNGK |
| | (human IgG1 | EYKCKVSNKALPAPIEKTISKAKGQPREPQVYTLPPSREEMTKNQVSLTC |
| | with L234A, | LVKGFYPSDIAVEWESNGQPENNYKTTPPVLDSDGSFFLYSKLTVDKSRW |
| | L235A, and | QQGNVFSCSVMHEALHNHYTQKSLSLSPG |
| | G237A) | |
| 45 | Antibody | RTVAAPSVFIFPPSDEQLKSGTASVVCLLNNFYPREAKVQWKVDNALQSG |
| | Kappa | NSQESVTEQDSKDSTYSLSSTLTLSKADYEKHKVYACEVTHQGLSSPVTK |
| | Constant | SFNRGEC |
| | Domain | |
| | (human) | |
| 46 | IL-2-G4Sx3-Fc | APTSSSTKKTQLQLEHLLLDLQMILNGINNYKNPKLTRMLTFKFYMPKKA |
| | | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 47 | IL-2 T3A- | APASSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHODWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 48 | IL-2 N88R- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISRINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHODWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 49 | IL-2 V69A, | APTSSSTKKTQLQLEHLLLDLQMILNGINNYKNPKLTRMLTFKFYMPKKA |
| | Q74P,-G4Sx3- | TELKHLQCLEEELKPLEEALNLAPSKNFHLRPRDLISNINVIVLELKGSE |
| | Fc | TTFMCEYADETATIVEFLNRWITFSQSIISTLTGGGGSGGGGSGGGGSDK |
| | | THTCPPCPAPEAAGAPSVELFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 50 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 51 | IL-2 N88R | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISRINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 52 | IL-2 N88D- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISDINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 53 | IL-2 L53I | APTSSSTKKTQLQLEHLLLDLQMILNGINNYKNPKLTRMLTFKFYMPKKA |
| | N88D V69A, | TEIKHLQCLEEELKPLEEALNLAPSKNFHLRPRDLISDINVIVLELKGSE |
| | Q74P,C125S- | TTFMCEYADETATIVEFLNRWITFSQSIISTLTGGGGSGGGGSGGGGSGG |
| | G4Sx4-Fc | GGSDKTHTCPPCPAPEAAGAPSVELFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 54 | IL-2 L56I | APTSSSTKKTQLQLEHLLLDLQMILNGINNYKNPKLTRMLTFKFYMPKKA |
| | N88D V69A, | TELKHIQCLEEELKPLEEALNLAPSKNFHLRPRDLISDINVIVLELKGSE |
| | Q74P, C125S- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 55 | IL-2 L80I | APTSSSTKKTQLQLEHLLLDLQMILNGINNYKNPKLTRMLTFKFYMPKKA |
| | N88D V69A, | TELKHLQCLEEELKPLEEALNLAPSKNFHIRPRDLISDINVIVLELKGSE |
| | C125S Q74P- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 56 | IL-2 L118I | APTSSSTKKTQLQLEHLLLDLQMILNGINNYKNPKLTRMLTFKFYMPKKA |
| | N88D V69A, | TELKHLQCLEEELKPLEEALNLAPSKNFHLRPRDLISDINVIVLELKGSE |
| | Q74P, C125S- | TTFMCEYADETATIVEFINRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 57 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx4-Fc | TTFMCEYADETATIVEFLNRWITFSQSIISTLTGGGGSGGGGSGGGGSGG |
| | | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 58 | Fc-G4S-IL-2 | DKTHTCPPCPAPELLGGPSVFLFPPKPKDTLMISRTPEVTCVVVDVSHED |
| | N88D V69A, | PEVKFNWYVDGVEVHNAKTKPREEQYGSTYRVVSVLTVLHQDWLNGKEYK |
| | Q74P | CKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQG |
| | | NVFSCSVMHEALHNHYTQKSLSLSPGGGGGSAPTSSSTKKTQLQLEHLLL |
| | | DLQMILNGINNYKNPKLTRMLTFKFYMPKKATELKHLQCLEEELKPLEEA |
| | | LNLAPSKNFHLRPRDLISDINVIVLELKGSETTFMCEYADETATIVEFLN |
| | | RWITFAQSIISTLT |
| 59 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELK |
| | C125S-G4Sx4- | GSETTFMCEYADETATIVEFX₄NRWITFSQSIISTLTGGGGSGGGGSGGG |
| | Fc, wherein | GSGGGGSDKTHTCPPCPAPEAAGAPSVELFPPKPKDTLMISRTPEVTCVV |
| | at least one | VDVSHEDPEVKFNWYVDGVEVHNAKTKPREEQYNSTYRVVSVLTVLHQDW |
| | of X₁, X₂, X₃, | LNGKEYKCKVSNKALPAPIEKTISKAKGQPREPQVYTLPPSREEMTKNQV |
| | and X4 is I | SLTCLVKGFYPSDIAVEWESNGQPENNYKTTPPVLDSDGSFFLYSKLTVD |
| | and the | KSRWQQGNVFSCSVMHEALHNHYTQKSLSLSPG |
| | remainder are | |
| | L or I. | |
| 60 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELK |
| | C125S, | GSETTFMCEYADETATIVEFX₄NRWITFSQSIISTLT |
| | wherein at | |
| | least one of | |
| | X₁, X₂, X₃, | |
| | and X4 is I | |
| | and the | |
| | remainder are | |
| | L or I. | |

In some embodiments, the sequences shown in the table or throughout comprise or do not comprise one or more mutations that correspond to positions L53, L56, L80, and L118. In some embodiments, the sequences shown in the table or throughout the present application comprise or do not comprise one or more mutations that correspond to positions L59I, L63I, I24L, L94I, L96I or L132I or other substitutions at the same positions. In some embodiments, the mutation is leucine to isoleucine. In some embodiments, the mutein does not comprise another mutation other than as shown or described herein. In some embodiments, the peptide comprises a sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56, SEQ ID NO: 57, SEQ ID NO: 58, SEQ ID NO: 59, or SEQ ID NO: 60.

In some embodiments, the protein comprises a IL-2 mutein as provided for herein. In some embodiments, a polypeptide is provided comprising SEQ ID NO: 59 or SEQ ID NO: 60, wherein at least one of X₁, X₂, X₃, and X₄ is I and the remainder of X₁, X₂, X₃, and X₄ are L or I. As used herein, and in reference to SEQ ID NO: 59 or SEQ ID NO: 60, X₁ indicates an amino acid at position 53 as compared to SEQ ID NO: 59 or SEQ ID NO: 60. As used herein, and in reference to SEQ ID NO: 59 or SEQ ID NO: 60, X₂ indicates an amino acid at position 56 as compared to SEQ ID NO: 59 or SEQ ID NO: 60. As used herein, and in reference to SEQ ID NO: 59 or SEQ ID NO: 60, X₃ indicates an amino acid at position 80 as compared to SEQ ID NO: 59 or SEQ ID NO: 60. As used herein, and in reference to SEQ ID NO: 59 or SEQ ID NO: 60, X₄ indicates an amino acid at position 118 as compared to SEQ ID NO: 59 or SEQ ID NO: 60. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

In some embodiments, the Fc portion of the fusion is not included. In some embodiments, the peptide consists essentially of a IL-2 mutein provided for herein. In some embodiments, the protein is free of a Fc portion.

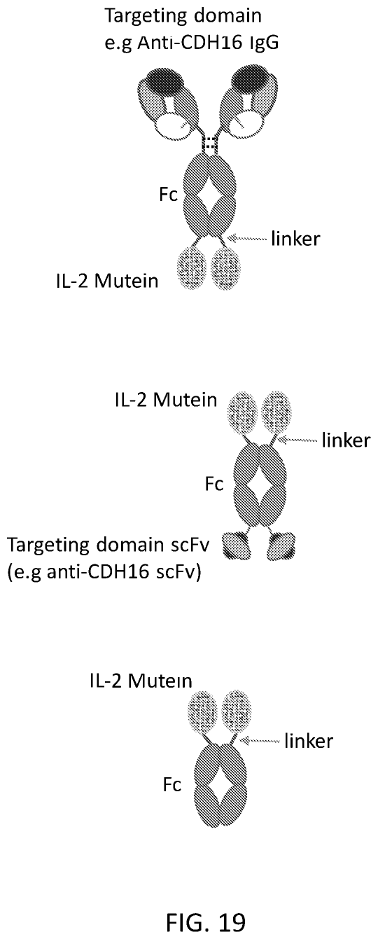

For illustrative purposes only, embodiments of IL-2 mutein fused with a Fc and with a targeting moiety are illustrated in FIG. 19. The targeting moiety is illustrated as an anti-CDH16 antibody, but that is for illustration purposes only and it can be replaced with another targeting moiety, including, but not limited to, for example OCT2. Similarly, the IL-2 mutein can be replaced with a PD-1 agonist or other type of effector molecule, such as CD39 or related family members of the ENTPD gene family.

The sequences are for illustrative purposes only and are not intended to be limiting. In some embodiments, the compound comprises an amino acid sequence of SEQ ID NO: 53, 54, 55, or 56. In some embodiments, the compound comprises an amino acid sequence of SEQ ID NO: 53, 54, 55, or 56 with or without a C125A or C125S mutation. In some embodiments, the residue at position 125 is C, S, or A. In some embodiments, the compound comprises an amino acid sequence of SEQ ID NO: 59 or SEQ ID NO: 60, wherein at least one of X₁, X₂, X₃, and X₄ is I and the remainder are L or I. In some embodiments, the protein comprises a IL-2 mutein as provided for herein. In some embodiments, a polypeptide is provided comprising SEQ ID NO: 59 or SEQ ID NO: 60, wherein at least one of X₁, X₂, X₃, and X₄ is I and the remainder are L or I. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

Each of the proteins may also be considered to have the C125S and the LALA and/or G237A mutations as provided for herein. The C125 substitution can also be C125A as described throughout the present application.

In some embodiments, an IL-2 mutein molecule comprises at least 60, 70, 80, 85, 90, 95, or 97% sequence identity or homology with a naturally occurring human IL-2 molecule, e.g., a naturally occurring IL-2 sequence disclosed herein or those that incorporated by reference.

As described herein the IL-2 muteins can be part of a bispecific molecule with a tethering moiety, such as an CDH16 or OCT2 that will target the IL-2 mutein to CDH16 or OCT2 expressing cell. As described herein, the bispecific molecule can be produced from two polypeptide chains.

In some embodiments, the anti-CDH16 or anti-OCT2 antibody, or any antigen binding fragment thereof, is linked to a CD39 effector domain. In some embodiments, the effector domain has a CD39 sequence as provided herein

### Auto-Immune Disorders

As provided herein, therapeutic compounds (polypeptides) and methods described herein can be used to treat a subject having, or at risk for having, an unwanted autoimmune response, that effect the kidney. Examples of such conditions are provided herein and include, but are not limited to, Goodpasture's Syndrome (anti-GBM disease), inflammatory renal disease, glomerulonephritis, nephritis, lupus, lupus nephritis, IgA nephritis, membranous nephropathy, membranoproliferative glomerulonephritis, acute kidney injury, and chronic kidney disease as well as any other autoimmune or inflammation disorders that can affect the kidneys. Other examples include, but are not limited to, focal segmented glomerular sclerosis (FSGS) and other diseases that can affect kidney, for example lupus nephritis, systemic scleroderma, membranous glomerular nephropathy (MGN), membranous nephropathy (MN), minimal change disease (MCD), IgA nephropathy, ANCA-associated vasculitis (AAV), Sjogren's syndrome, and Scleroderma, systemic sclerosis (SSc).

Other examples of autoimmune disorders and diseases that can be treated with the compounds, compositions, and polypeptides provided herein include, but are not limited to, anti-glomerular basement membrane nephritis, lupus nephritis, membranous glomerulonephropathy, chronic kidney disease (“CKD”),

In some embodiments, the treatment minimizes rejection of, minimizes immune effector cell mediated damage to, prolongs the survival of subject tissue undergoing, or a risk for, autoimmune attack.

In some embodiments, methods are provided herein for activating and/or increasing Tregs in the kidney. In some embodiments, the Tregs are activated or increased in the kidney tubules. In some embodiments, the Treg activation and/or expansions is selective (specific) to the kidney, such as in the kidney tubules. This can be done, for example, by tethering the active moiety, such as the IL-2 mutein or other active moieties provided for herein, to a kidney, or kidney-tubule, specific targeting moiety. In some embodiments, the methods comprise administering to the subject, a polypeptide, composition, or pharmaceutical composition as provided for herein.

### Therapeutic Compounds

A therapeutic compound, which can be a polypeptide, comprises a specific targeting moiety functionally associated with an effector binding/modulating moiety. In some embodiments, the specific targeting moiety (e.g. anti-CDH16 antibody or anti-OCT2 antibody) and effector binding/modulating moiety are linked to one another by a covalent or noncovalent bond, e.g., a covalent or non-covalent bond directly linking the one to the other. In other embodiments, a specific targeting moiety and effector binding/modulating moiety are linked, e.g., covalently or noncovalently, through a linker moiety. For example, in the case of a fusion polypeptide, a polypeptide sequence comprising the specific targeting moiety and a polypeptide sequence can be directly linked to one another or linked through one or more linker sequences. In some embodiments, the linker moiety comprises a polypeptide. Linkers are not, however, limited to polypeptides. In some embodiments, a linker moiety comprises other backbones, e.g., a non-peptide polymer, e.g., a PEG polymer. In some embodiments, a linker moiety can comprise a particle, e.g., a nanoparticle, e.g., a polymeric nanoparticle. In some embodiments, a linker moiety can comprise a branched molecule, or a dendrimer.

In some embodiments, a therapeutic compound comprises a polypeptide comprising a specific targeting moiety covalently or non-covalently conjugated to an effector binding/modulating moiety. In some embodiments, a therapeutic molecule comprises a fusion protein having comprising a specific targeting moiety fused, e.g., directly or through a linking moiety comprising one or more amino acid residues, to an effector binding/modulating moiety. In some embodiments, a therapeutic molecule comprises a polypeptide comprising a specific targeting moiety linked by a non-covalent bond or a covalent bond, e.g., a covalent bond other than a peptide bond, e.g., a sulfhydryl bond, to an effector binding/modulating moiety.

In some embodiments, a therapeutic compound comprises polypeptide, e.g., a fusion polypeptide, comprising:
1.a) a specific targeting moiety comprising a target specific binding polypeptide;
1.b) a specific targeting moiety comprising a target ligand binding molecule;
1.c) a specific targeting moiety comprising an antibody molecule;
1.d) a specific targeting moiety comprising a single chain antibody molecule, e.g., a scFv domain; or
1.e) a specific targeting moiety comprising a first of the light or heavy chain variable region of an antibody molecule, and wherein the other variable region is covalently or non-covalently associated with the first;
and
2.a) an effector binding/modulating moiety comprising an effector specific binding polypeptide;
2.b) an effector binding/modulating moiety comprising an effector ligand binding molecule;
2.c) an effector binding/modulating moiety comprising an antibody molecule;
2.d) an effector binding/modulating moiety comprising a single chain antibody molecule, e.g., a scFv domain; or
2.e) an effector binding/modulating moiety comprising a first of the light or heavy chain variable region of an antibody molecule, and wherein the other variable region is covalently or non-covalently associated with the first.

In some embodiments, a therapeutic compound comprises 1.a and 2.a.

In some embodiments, a therapeutic compound comprises 1.a and 2.b.

In some embodiments, a therapeutic compound comprises 1.a and 2.c.

In some embodiments, a therapeutic compound comprises 1.a and 2.d.

In some embodiments, a therapeutic compound comprises 1.a and 2.e.

In some embodiments, a therapeutic compound comprises 1.b and 2.a.

In some embodiments, a therapeutic compound comprises 1.b and 2.b.

In some embodiments, a therapeutic compound comprises 1.b and 2.c.

In some embodiments, a therapeutic compound comprises 1.b and 2.d.

In some embodiments, a therapeutic compound comprises 1.b and 2.e.

In some embodiments, a therapeutic compound comprises 1.c and 2.a.

In some embodiments, a therapeutic compound comprises 1.c and 2.b.

In some embodiments, a therapeutic compound comprises 1.c and 2.c.

In some embodiments, a therapeutic compound comprises 1.c and 2.d.

In some embodiments, a therapeutic compound comprises 1.c and 2.e.

In some embodiments, a therapeutic compound comprises 1.d and 2.a.

In some embodiments, a therapeutic compound comprises 1.d and 2.b.

In some embodiments, a therapeutic compound comprises 1.d and 2.c.

In some embodiments, a therapeutic compound comprises 1.d and 2.d.

In some embodiments, a therapeutic compound comprises 1.d and 2.e.

In some embodiments, a therapeutic compound comprises 1.e and 2.a.

In some embodiments, a therapeutic compound comprises 1.e and 2.b.

In some embodiments, a therapeutic compound comprises 1.e and 2.c.

In some embodiments, a therapeutic compound comprises 1.e and 2.d.

In some embodiments, a therapeutic compound comprises 1.e and 2.e.

In these non-limiting examples, the targeting moiety can be the anti-CDH16 antibody and the effector binding/modulating moiety can be the PD-1 agonist, the IL-2 mutein, or the CD39 effector domain. In some embodiments, the targeting moiety can be the anti-OCT2 antibody and the effector binding/modulating moiety can be the PD-1 agonist, the IL-2 mutein, or the CD39 effector, as provided herein. Non-limiting examples of each are provided herein. Therapeutic compounds disclosed herein can, for example, comprise a plurality of effector binding/modulating and specific targeting moieties. Any suitable linker or platform can be used to present the plurality of moieties. The linker is typically coupled or fused to one or more effector binding/modulating and targeting moieties.

In some embodiments, two (or more) linkers associate, either covalently or non-covalently, e.g., to form a hetero- or homodimeric therapeutic compound. For example, the linker can comprise an Fc region and two Fc regions associate with one another. In some embodiments of a therapeutic compound comprising two linker regions, the linker regions can self associate, e.g., as two identical Fc regions. In some embodiments of a therapeutic compound comprising two linker regions, the linker regions are not capable of, or not capable of substantial, self association, e.g., the two Fc regions can be members of a knob and hole pair.

Non-limiting exemplary configurations of therapeutic compounds comprise the following (e.g., in N to C terminal order):
R1-Linker Region A-R2 or
R3-Linker Region B-R4,
wherein,
R1, R2, R3, and R4, each independently comprises an effector binding/modulating moiety, e.g., anti-PD-1 molecule (antibody), IL-2 mutein, CD39, or is absent, provided that at least one of R1 and R2 is not absent, and at least one of R3 and R4 is not absent;
Linker Region A and Linker Region B comprise moieties that can associate with one another, e.g., Linker A and Linker B each comprises an Fc moiety provided that an effector binding/modulating moiety and a specific targeting moiety are present.

In some embodiments, the polypeptide having the formula of R1-Linker Region A-R2 and the polypeptide having the formula of R3-Linker Region B-R4 interact with one another to form a polypeptide complex.

In some embodiments, the polypeptide having the formula of R1-Linker Region A-R2 and the polypeptide having the formula of R3-Linker Region B-R4 do not interact with one another to form a polypeptide complex.

In some embodiments:
R1 comprises an effector binding/modulating moiety, e.g., anti-PD-1 molecule, IL-2 mutein, CD39, or is absent;
R2 comprises a specific targeting moiety, or is absent;
R3 comprises an effector binding/modulating moiety, e.g., anti-PD-1 molecule, IL-2 mutein, CD39, or is absent;
R4 comprises a specific targeting moiety, or is absent;
Linker Region A and Linker Region B comprise moieties that can associate with one another, e.g., Linker A and Linker B each comprises an Fc moiety, provided that one of R1 or R3 is present and one of R2 or R4 is present.

In some embodiments:
R1 comprises a specific targeting moiety, or is absent; R2 comprises an effector binding/modulating moiety, e.g., anti-PD-1 molecule, IL-2 mutein, CD39, or is absent;
R3 comprises a specific targeting moiety, or is absent;
R4 comprises an effector binding/modulating moiety, e.g., anti-PD-1 molecule, IL-2 mutein, CD39, or is absent;
Linker Region A and Linker Region B comprise moieties that can associate with one another, e.g., Linker A and Linker B each comprises an Fc moiety, provided that one of R1 or R3 is present and one of R2 or R4 is present.

Non-limiting examples include, but are not limited to:

| | Linker | | | Linker | | |
|---|---|---|---|---|---|---|
| R1 | Region A | R2 | R3 | Region B | R4 | Other |
| HCVR and | Fc Region | fcFv | HCVR and | Fc Region | scFv | Self Pairing |
| LCVR | | | LCVR | | | Linker Regions |
| HCVR and | Fc Region | fcFv | HCVR and | Fc Region | scFv | Non-Self |
| LCVR | | | LCVR | | | Pairing linker |
| | | | | | | regions |
| HCVR and | Fc Region | fcFv | HCVR and | Fc Region | scFv | Self Pairing |
| LCVR (or | | | LCVR (or | | | Linker Regions |
| absent) | | | absent) | | | One of R1 or |
| | | | | | | R3 is absent. |
| HCVR and | Fc Region | fcFv | HCVR and | Fc Region | scFv | Non-Self |
| LCVR (or | | | LCVR (or | | | Pairing Linker |
| absent) | | | absent) | | | Regions |
| | | | | | | One of R1 or |
| | | | | | | R3 is absent. |
| HCVR and | Fc Region | fcFv (or | HCVR and | Fc Region | scFv (or | Self Pairing |
| LCVR | | absent) | LCVR | | absent) | linker regions |
| | | | | | | One of R2 or |
| | | | | | | R4 is absent. |
| HCVR and | Fc Region | fcFv (or | HCVR and | Fc Region | scFv (or | Non-Self |
| LCVR | | absent) | LCVR | | absent) | Pairing linker |
| | | | | | | regions |
| | | | | | | One of R2 or |
| | | | | | | R4 is absent. |
| HCVR and | Fc Region | fcFv | HCVR and | Fc Region | scFv | Self Pairing |
| LCVR | | | LCVR | | | Linker Regions |
| | | | | | | R1 and R3 are |
| | | | | | | the same |
| HCVR and | Fc Region | fcFv | HCVR and | Fc Region | scFv | Non-Self |
| LCVR | | | LCVR | | | Pairing linker |
| | | | | | | regions |
| | | | | | | R1 and R3 are |
| | | | | | | different |
| HCVR and | Fc Region | fcFv | HCVR and | Fc Region | scFv | Self Pairing |
| LCVR | | | LCVR | | | Linker Regions |
| | | | | | | R2 and R4 are |
| | | | | | | the same |
| HCVR and | Fc Region | fcFv | HCVR and | Fc Region | scFv | Non-Self |
| LCVR | | | LCVR | | | Pairing linker |
| | | | | | | regions |
| | | | | | | R2 and R4 are |
| | | | | | | different |

HCVR and LCVR: refers to an moiety comprising an antigen binding portion of a heavy and light chain variable region, typically with the heavy chain fused to the Linker region.
Self pairing: wherein a liker region can pair with itself, e.g., an Fc region that can pair a copy of itself.
Non-self pairing: wherein a Linker Region does not pair with itself, or does not substantially pair with itself, e.g., an Fc region does not, or does not significantly pair with itself, e.g., wherein Linker Region A and Linker Region B are members of a knob and hole pair.

In some embodiments:
R1, R2, R3 and R4 each independently comprise: an effector binding modulating moiety that activates an inhibitory receptor on an immune cell, e.g., a T cell or a B cell, e.g., a PD-L1 molecule or a functional anti-PD-1 antibody molecule (an agonist of PD-1), a specific targeting moiety, or is absent; provided that an effector binding moiety and a specific targeting moiety are present.

In some embodiments, Linker A and Linker B comprise Fc moieties (e.g., self pairing Fc moieties).

In some embodiments:
R1 and R3 independently comprise an effector binding modulating moiety that activates an inhibitory receptor on an immune cell, e.g., a T cell or a B cell, e.g., a PD-L1 molecule or an functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self pairing Fc moieties).

In some embodiments:
R1 and R3 independently comprise a functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a functional anti-PD-1 antibody molecule (an agonist of PD-1), e.g., an scFv molecule.

In some embodiments, Linker A and Linker B comprise Fc moieties (e.g., self pairing Fc moieties).

In some embodiments:
R1 and R3 independently comprise a PD-L1 molecule (an agonist of PD-1); and
R2 and R4 independently comprise specific targeting moieties, e.g., scFv molecules against a tissue antigen; and in some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a PD-L1 molecule (an agonist of PD-1).

In some embodiments, Linker A and Linker B comprise Fc moieties (e.g., self pairing Fc moieties).

In some embodiments:
R1 and R3 independently comprise a CD39 molecule or a CD73 molecule; and
R2 and R4 independently comprise specific targeting moieties, e.g., scFv molecules against a tissue antigen.

In some embodiments, Linker A and Linker B comprise Fc moieties (e.g., self pairing Fc moieties or Fc moieties that do not, or do not substantially self pair).

In some embodiments:
R1 and R3 each comprises a CD39 molecule; and
R2 and R4 independently comprise specific targeting moieties, e.g., scFv molecules against a tissue antigen; and in some embodiments, Linker A and Linker B comprise Fc moieties (e.g., self pairing Fc moieties or Fc moieties that do not, or do not substantially self pair).

In some embodiments:

In an embodiment:
R1, R2, R3 and R4 each independently comprise: an IL-2 mutein molecule; a specific targeting moiety, or is absent;
provided that an IL-2 mutein molecule and a specific targeting moiety are present.

In an embodiment, Linker A and Linker B comprise Fc moieties (e.g., self pairing Fc moieties or Fc moieties that do not, or do not substantially self pair).

In an embodiment:
R1 and R3 each comprise an IL-2 mutein molecule; and
R2 and R4 independently comprise specific targeting moieties, e.g., scFv molecules against a tissue antigen.

In an embodiment Linker A and Linker B comprise Fc moieties (e.g., self pairing Fc moieties or Fc moieties that do not, or do not substantially self pair).

In some embodiments, one of R1, R2, R3, and R4 comprises an SM binding/modulating moiety, e.g., a CD39 molecule. In some embodiments, one of R1, R2, R3, and R4 comprises an entity that binds, activates, or maintains, a regulatory immune cell, e.g., a Treg cell or a Breg cell, for example, an IL-2 mutein molecule.

In some embodiments, one of R1, R2, R3, and R4 comprises an agonistic anti-PD-1 antibody. In some embodiments, the PD-1 antibody is replaced with a IL-2 mutein molecule. In some embodiments, one of R1, R2, R3, and R4 comprises an agonistic anti-PD-1 antibody, one comprises an HLA-G molecule, and one comprises CD39 molecule. In some embodiments, the PD-1 antibody is replaced with a IL-2 mutein molecule.

In some embodiments, one of R1 and R2 is an IL-2 mutein and one of R1 and R2 is an anti-CDH16 antibody or an anti-OCT2 antibody. In some embodiments, R1 is an IL-2 mutein and R2 is anti-CDH16 antibody or an anti-OCT2 antibody. In some embodiments, R1 is an anti-CDH16 antibody or an anti-OCT2 antibody and R2 is an IL-2 mutein. In some embodiments, one of R3 and R4 is an IL-2 mutein and one of R3 and R4 is an anti-CDH16 antibody or an anti-OCT2 antibody. In some embodiments, R3 is an IL-2 mutein and R4 is an anti-CDH16 antibody or an anti-OCT2 antibody. In some embodiments, R3 is anti-CDH16 antibody or an anti-OCT2 antibody and one R4 is an IL-2 mutein.

In some embodiments, one of R1 and R2 is an anti-PD-1 antibody and one of R1 and R2 is an anti-CDH16 antibody or an anti-OCT2 antibody. In some embodiments, R1 is an anti-PD-1 antibody and R2 is anti-CDH16 antibody or an anti-OCT2 antibody. In some embodiments, R1 is an anti-CDH16 antibody or an anti-OCT2 antibody and R2 is an anti-PD-1 antibody. In some embodiments, one of R3 and R4 is an anti-PD-1 antibody and one of R3 and R4 is an anti-CDH16 antibody or an anti-OCT2 antibody. In some embodiments, R3 is an anti-PD-1 antibody and R4 is an anti-CDH16 antibody or an anti-OCT2 antibody. In some embodiments, R3 is anti-CDH16 antibody or an anti-OCT2 antibody and one R4 is an anti-PD-1 antibody.

In some embodiments, one of R1 and R2 is a CD39 Effector Domain and one of R1 and R2 is an anti-CDH16 antibody or an anti-OCT2 antibody. In some embodiments, R1 is a CD39 Effector Domain and R2 is anti-CDH16 antibody or an anti-OCT2 antibody. In some embodiments, R1 is an anti-CDH16 antibody or an anti-OCT2 antibody and R2 is a CD39 Effector Domain. In some embodiments, one of R3 and R4 is a CD39 Effector Domain and one of R3 and R4 is an anti-CDH16 antibody or an anti-OCT2 antibody. In some embodiments, R3 is a CD39 Effector Domain and R4 is an anti-CDH16 antibody or an anti-OCT2 antibody. In some embodiments, R3 is anti-CDH16 antibody or an anti-OCT2 antibody and one R4 is a CD39 Effector Domain.

In some embodiments, the targeting moiety of the molecules provided for herein are a CDH16 targeting moiety, such as an antibody.

### Linker Regions

As discussed elsewhere herein specific targeting and effector binding/modulating moieties can be linked by linker regions. Any linker region described herein can be used as a linker. For example, Linker Regions A and B can comprise Fc regions. In some embodiments, a therapeutic compound comprises a Linker Region that can self-associate. In some embodiments, a therapeutic compound comprises a Linker Region that has a moiety that minimizes self association, and typically Linker Region A and Linker Region B are heterodimers. Linkers also include glycine/serine linkers. In some embodiments, the linker can comprise one or more repeats of GGGGS (SEQ ID NO: 23). In some embodiments, the linker comprises 1, 2, 3, 4, or 5 repeats of SEQ ID NO: 23. In some embodiments, the linker comprises of GGGGS (SEQ ID NO: 23), GGGGSGGGGSGGGGSGGGGS (SEQ ID NO: 22), GGGGSGGGGS (SEQ ID NO: 484), or GGGGSGGGGSGGGGS (SEQ ID NO: 30). In some embodiments, the linker comprises of GGGGSEGGGSEGGGSEGGGSE (SEQ ID NO: 71), GGGSEGGGSEGGGSEGGGSE (SEQ ID NO: 72), GGGSEGGGSEGGGSE (SEQ ID NO: 73), AEEEKAEEEKAEEEKAEEEK (SEQ ID NO: 74), GGGSKGGGSKGGGSK (SEQ ID NO: 75), GSAGKGSAGKGSAGK (SEQ ID NO: 76), GGGSKGGGSKGGGSK (SEQ ID NO: 77), GSAGK (SEQ ID NO: 78), GS, GAGGGSKGGGSKGGGSK (SEQ ID NO: 79), GSAGKGSAGKGSAGK (SEQ ID NO: 80), GGGSK (SEQ ID NO: 81), AEEEK (SEQ ID NO: 82), GGSSGSGSGSTGTSSSGTGTSAGTTGTSASTSGSGSGGGGGSGGGGSAGGTATAGASSG S (SEQ ID NO: 83), These linkers can be used in any of the therapeutic compounds or compositions provided herein.

The linker region can comprise a Fc region that has been modified (e.g., mutated) to produce a heterodimer. In some embodiments, the CH3 domain of the Fc region can be mutated. Examples of such Fc regions can be found in, for example, U.S. Pat. No. 9,574,010, which is hereby incorporated by reference in its entirety. The Fc region as defined herein comprises a CH3 domain or fragment thereof, and may additionally comprise one or more addition constant region domains, or fragments thereof, including hinge, CH1, or CH2. It will be understood that the numbering of the Fc amino acid residues is that of the EU index as in Kabat et al 1991, NIH Publication 91-3242, National Technical Information Service, Springfield, Va. The “EU index as set forth in Kabat” refers to the EU index numbering of the human IgG1 Kabat antibody. For convenience, Table B of U.S. Pat. No. 9,574,010 provides the amino acids numbered according to the EU index as set forth in Kabat of the CH2 and CH3 domain from human IgG1, which is hereby incorporated by reference. Table 1.1 of U.S. Pat. No. 9,574,010 provides mutations of variant Fc heterodimers that can be used as linker regions. Table 1.1 of U.S. Pat. No. 9,574,010 is hereby incorporated by reference.

In some embodiments, the Linker Region A comprises a first CH3 domain polypeptide and a the Linker Region B comprises a second CH3 domain polypeptide, the first and second CH3 domain polypeptides independently comprising amino acid modifications as compared to a wild-type CH3 domain polypeptide, wherein the first CH3 domain polypeptide comprises amino acid modifications at positions T350, L351, F405, and Y407, and the second CH3 domain polypeptide comprises amino acid modifications at positions T350, T366, K392 and T394, wherein the amino acid modification at position T350 is T350V, T3501, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V, or T366M; the amino acid modification at position K392 is K392F, K392L or K392M; and the amino acid modification at position T394 is T394W, and wherein the numbering of amino acid residues is according to the EU index as set forth in Kabat.

In some embodiments, the amino acid modification at position K392 is K392M or K392L. In some embodiments, the amino acid modification at position T350 is T350V. In some embodiments, the first CH3 domain polypeptide further comprises one or more amino acid modifications selected from Q347R and one of S400R or S400E. In some embodiments, the second CH3 domain polypeptide further comprises one or more amino acid modifications selected from L351Y, K360E, and one of N390R, N390D or N390E. In some embodiments, the first CH3 domain polypeptide further comprises one or more amino acid modifications selected from Q347R and one of S400R or S400E, and the second CH3 domain polypeptide further comprises one or more amino acid modifications selected from L351Y, K360E, and one of N390R, N390D or N390E. In some embodiments, the amino acid modification at position T350 is T350V. In some embodiments, the amino acid modification at position F405 is F405A. In some embodiments, the amino acid modification at position Y407 is Y407V. In some embodiments, the amino acid modification at position T366 is T366L or T366I. In some embodiments, the amino acid modification at position F405 is F405A, the amino acid modification at position Y407 is and Y407V, the amino acid modification at position T366 is T366L or T366I, and the amino acid modification at position K392 is K392M or K392L. In some embodiments, the first CH3 domain polypeptide comprises the amino acid modifications T350V, L351Y, S400E, F405V and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405T and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405S and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, L351Y, T366L, N390R, K392M and T394W. In some embodiments, the first CH3 domain polypeptide comprises the amino acid modifications Q347R, T350V, L351Y, S400E, F405A and Y407V, and the second CH3 domain polypeptide comprises the amino acid modifications T350V, K360E, T366L, N390R, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390D, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390E, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392L and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392F and T394W.

In some embodiments, an isolated heteromultimer comprising a heterodimeric CH3 domain comprising a first CH3 domain polypeptide and a second CH3 domain polypeptide, the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70° C. or greater and a purity greater than about 90%, and wherein the numbering of amino acid residues is according to the EU index as set forth in Kabat.

In some embodiments, the Linker Region A comprises a first CH3 domain polypeptide and a t Linker Region B comprises a second CH3 domain polypeptide, wherein the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70 C. or greater and a purity greater than about 90%, and wherein the numbering of amino acid residues is according to the EU index as set forth in Kabat. In some embodiments, the amino acid modification at position F405 is F405A. In some embodiments, the amino acid modification at position T366 is T366I or T366L. In some embodiments, the amino acid modification at position Y407 is Y407V. In some embodiments, the amino acid modification at position F405 is F405A, the amino acid modification at position Y407 is Y407V, the amino acid modification at position T366 is T366I or T366L, and the amino acid modification at position K392 is K392L or K392M. In some embodiments, the amino acid modification at position F405 is F405A, the amino acid modification at position Y407 is Y407V, the amino acid modification at position T366 is T366L, and the amino acid modification at position K392 is K392M. In some embodiments, the amino acid modification at position F405 is F405A, the amino acid modification at position Y407 is Y407V, the amino acid modification at position T366 is T366L, and the amino acid modification at position K392 is K392L. In some embodiments, the amino acid modification at position F405 is F405A, the amino acid modification at position Y407 is Y407V, the amino acid modification at position T366 is T366I, and the amino acid modification at position K392 is K392M. In some embodiments, the amino acid modification at position F405 is F405A, the amino acid modification at position Y407 is Y407V, the amino acid modification at position T366 is T366I, and the amino acid modification at position K392 is K392L. In some embodiments, the first CH3 domain polypeptide further comprises an amino acid modification at position S400 selected from S400D and S400E, and the second CH3 domain polypeptide further comprises the amino acid modification N390R. In some embodiments, the amino acid modification at position F405 is F405A, the amino acid modification at position Y407 is Y405V, the amino acid modification at position S400 is S400E, the amino acid modification at position T366 is T366L, and the amino acid modification at position K392 is K392M.

In some embodiments, the modified first and second CH3 domains are comprised by an Fc construct based on a type G immunoglobulin (IgG). The IgG can be an IgG1, IgG2, IgG3, or IgG4.

Other Linker Region A and Linger Region B comprising variant CH3 domains are described in U.S. Pat. Nos. 9,499,634 and 9,562,109, each of which is incorporated by reference in its entirety.

A Linker Region A and Linker Region B can be complementary fragments of a protein, e.g., a naturally occurring protein such as human serum albumin. In embodiments, one of Linker Region A and Linker Region B comprises a first, e.g., an N-terminal fragment of the protein, e.g., hSA, and the other comprises a second, e.g., a C-terminal fragment of the protein, e.g., has. In an embodiment the fragments comprise an N-terminal and a C-terminal fragment. In an embodiment the fragments comprise two internal fragments. Typically the fragments do not overlap. In an embodiment the first and second fragment, together, provide the entire sequence of the original protein, e.g., hSA. The first fragment provides a N-terminus and a C-terminus for linking, e.g., fusing, to other sequences, e.g., sequences of R1, R2, R3, or R4 (as defined herein).

The Linker Region A and the Linker Region B can be derived from albumin polypeptide. In some embodiments, the albumin polypeptide is selected from native human serum albumin polypeptide and human alloalbumin polypeptide. The albumin polypeptide can be modified such that the Linker Region A and Linker Region B interact with one another to form heterodimers. Examples of modified albumin polypeptides are described in U.S. Pat. Nos. 9,388,231 and 9,499,605, each of which is hereby incorporated by reference in its entirety.

Accordingly, provided herein are multifunctional heteromultimer proteins of the formula R1-Linker Region A-R2 and R3-Linker Region B-R4, wherein the Linker Region A and Linker Region B form a heteromultimer. In some embodiments, the Linker Region A comprises a first polypeptide and the Linker Region B comprises a second polypeptide; wherein each of said first and second polypeptides comprises an amino acid sequence comprising a segment of an albumin polypeptide selected from native human serum albumin polypeptide and human alloalbumin polypeptide; wherein said first and second polypeptides are obtained by segmentation of said albumin polypeptide at a segmentation site, such that the segmentation results in a deletion of zero to 3 amino acid residues at the segmentation site; wherein said first polypeptide comprises at least one mutation selected from A194C, L198C, W214C, A217C, L331C and A335C, and said second polypeptide comprises at least one mutation selected from L331C, A335C, V343C, L346C, A350C, V455C, and N458C; and wherein said first and second polypeptides self-assemble to form a quasi-native structure of the monomeric form of the albumin polypeptide.

In some embodiments, the segmentation site resides on a loop of the albumin polypeptide that has a high solvent accessible surface area (SASA) and limited contact with the rest of the albumin structure. In some embodiments, the segmentation results in a complementary interface between the transporter polypeptides. These segmentation sites are described, for example, in U.S. Pat. No. 9,388,231, which is hereby incorporated by reference in its entirety.

In some embodiments, the first polypeptide comprises residues 1-337 or residues 1-293 of the albumin polypeptide with one or more of the mutations described herein. In some embodiments, the second polypeptide comprises residues of 342-585 or 304-585 of the albumin polypeptide with one or more of the mutations described herein. In some embodiments, the first polypeptide comprises residues 1-339, 1-300, 1-364, 1-441, 1-83, 1-171, 1-281, 1-293, 1-114, 1-337, or 1-336 of the albumin protein. In some embodiments, the second polypeptide comprises residues 301-585, 365-585, 442-585, 85-585, 172-585, 282-585, or 115-585, 304-585, 340-585, or 342-585 of the albumin protein.

In some embodiments, the first and second polypeptide comprise the residues of the albumin protein as shown in the table below. The sequence of the albumin protein is described below.

| First Polypeptide Residues | Second Polypeptide Residues |
|---|---|
| 1-300 | 301-585 |
| 1-364 | 365-585 |
| 1-441 | 442-585 |
| 1-83 | 85-585 |
| 1-171 | 172-585 |
| 1-281 | 282-585 |
| 1-114 | 115-585 |
| 1-339 | 340-585 |
| 1-337 | 342-585 |
| 1-293 | 304-585 |
| 1-336 | 342-585 |

In some embodiments, the first and second polypeptides comprise a linker that can form a covalent bond with one another, such as a disulfide bond. A non-limiting example of the linker is a peptide linker. In some embodiments, the peptide linker comprises GGGGS (SEQ ID NO: 23). The linker can be fused to the C-terminus of the first polypeptide and the N-terminus of the second polypeptide. The linker can also be used to attach the moieties described herein without abrogating the ability of the linkers to form a disulfide bond. In some embodiments, the first and second polypeptides do not comprise a linker that can form a covalent bond. In some embodiments, the first and second polypeptides have the following substitutions.

| First Polypeptide Substitution | Second Polypeptide Substitution |
|---|---|
| A217C | V343C |
| L331C | A350C |
| A217C | L346C |
| W214C | V343C |
| A335C | L346C |
| L198C | V455C |
| A217C | A335C |
| A217C | L331C |
| L198C | N458C |
| A194C | V455C |

The sequence of the albumin polypeptide can be the sequence of human albumin as shown, in the post-protein form with the N-terminal signaling residues removed

(MKWVTFISLLFLFSSAYSRGVFRR, SEQ ID NO: 84)
(human albumin, SEQ ID NO: 85)
DAHKSEVAHRFKDLGEENFKALVLIAFAQYLQQCPFEDHV
KLVNEVTEFAKTCVADESAENCDKSLHTLFGDKLCTVATL
RETYGEMADCCAKQEPERNECFLQHKDDNPNLPRLVRPEV
DVMCTAFHDNEETFLKKYLYEIARRHPYFYAPELLFFAKRY
KAAFTECCQAADKAACLLPKLDELRDEGKASSAKQRLKCA
SLQKFGERAFKAWAVARLSQRFPKAEFAEVSKLVTDLTKV
HTECCHGDLLECADDRADLAKYICENQDSISSKLKECCEKP
LLEKSHCIAEVENDEMPADLPSLAADFVESKDVCKNYAEA
KDVFLGMFLYEYARRHPDYSVVLLLRLAKTYETTLEKCCA
AADPHECYAKVFDEFKPLVEEPQNLIKQNCELFEQLGEYKF
QNALLVRYTKKVPQVSTPTLVEVSRNLGKVGSKCCKHPEA
KRMPCAEDYLSVVLNQLCVLHEKTPVSDRVTKCCTESLVN
RRPCFSALEVDETYVPKEFNAETFTFHADICTLSEKERQIKK
QTALVELVKHKPKATKEQLKAVMDDFAAFVEKCCKADDK
ETCFAEEGKKLVAASQAALGL

In some embodiments, the Linker Region A and the Linker Region B form a heterodimer as described herein.

In some embodiments, the polypeptide comprises at the N-terminus an antibody comprised of F(ab′)2 on an IgG1 Fc backbone fused with scFvs on the C-terminus of the IgG Fc backbone. In some embodiments, the IgG Fc backbone is a IgG1 Fc backbone. In some embodiments, the IgG1 backbone is replaced with a IgG4 backbone, IgG2 backbone, or other similar IgG backbone. The IgG backbones described in this paragraph can be used throughout this application where a Fc region is referred to as part of the therapeutic compound. Thus, in some embodiments, the antibody comprised of F(ab′)2 on an IgG1 Fc backbone can be an anti-CDH16 antibody or an anti-OCT2 antibody on an IgG1 Fc or any other targeting moiety or effector binding/modulating moiety provided herein. In some embodiments, the scFV segments fused to the C-terminus could be an anti-PD-1 antibody, if the N-terminus region is an anti-CDH16 antibody or an anti-OCT2 antibody, if the N-terminus region is an anti-PD-1 antibody. In this non-limiting example, the N-terminus can be the targeting moiety, such as any one of the ones provided for herein, and the C-terminus can be the effector binding/modulating moiety, such as any of the ones provided for herein. Alternatively, in some embodiments, the N-terminus can be the effector binding/modulating moiety, such as any one of the ones provided for herein, and the C-terminus can be the targeting moiety, such as any of the ones provided for herein.

In some embodiments, the N-terminus can be the targeting moiety, such as any one of the ones provided for herein, and the C-terminus can be the effector binding/modulating moiety, such as any of the ones provided for herein.

In some embodiments, the therapeutic compound comprises two polypeptides that homodimerize. In some embodiments, the N-terminus of the polypeptide comprises an effector binding/modulating moiety that is fused to a human IgG1 Fc domain (e.g., CH2 and/or CH3 domains). In some embodiments, the C-terminus of the Fc domain is another linker that is fused to the targeting moiety. Thus, in some embodiments, the molecule could be represented using the formula of R1-Linker A-Fc Region-Linker B-R2, wherein R1 can be an effector binding/modulating moiety, R2 is a targeting moiety, Linker A and Linker B are independently linkers as provided for herein. In some embodiments, Linker 1 and Linker 2 are different.

In some embodiments, the molecule could be represented using the formula of R1-Linker A-Fc Region-Linker B-R2, wherein R1 can be a targeting moiety, R2 is an effector binding/modulating moiety, Linker A and Linker B are independently linkers as provided for herein. In some embodiments, Linker A and Linker B are different. The linkers can be chosen from the non-limiting examples provided for herein. In some embodiments, R1 and R2 are independently selected from F(ab′)2 and scFV antibody domains. In some embodiments, R1 and R2 are different antibody domains. In some embodiments, the scFV is in the VL-VH domain orientation.

In some embodiments, the therapeutic compound is a bispecific antibody. In some embodiments, the bispecific antibodies are comprised of four polypeptide chains comprising the following:
Chain 1: nt-VH1-CH1-CH2-CH3-Linker A-scFv [VL2-Linker B-VH2]-ct
Chain 2: nt-VH1-CH1-CH2-CH3-Linker A-scFv [VL2-Linker B-VH2]-ct
Chain 3: nt-VL1-CL-ct
Chain 4: nt-VL1-CL-ct,
wherein chains 1 and 2 are identical to each other, and chains 3 and 4 are identical to each other,
wherein chain 1 forms a homodimer with chain 2; and chain 3 and 4 associate with chain 1 and chain 2. That is, when each light chain associates with each heavy chain, VL1 associates with VH1 and CL associates with CH1 to form two functional Fab units. Without being bound to any particular theory, each scFv unit is intrinsically functional since VL2 and VH2 are covalently linked in tandem with a linker as provided herein (e.g., GGGGSG (SEQ ID NO: 23), GGGGSGGGGSGGGGSGGGGS (SEQ ID NO: 22), GGGGSGGGGS (SEQ ID NO: 484), or GGGGSGGGGSGGGGS (SEQ ID NO: 30). The sequences of Linker A and Linker B, which are independent of one another can be the same or different and as otherwise described throughout the present application. Thus, in some embodiments, Linker A comprises GGGGS (SEQ ID NO: 23), or two repeats thereof, GGGGGGGGSGGGGS (SEQ ID NO: 30), or GGGGSGGGGSGGGGSGGGGS (SEQ ID NO: 22). In some embodiments, Linker B comprises GGGGS (SEQ ID NO: 23), or two repeats thereof, GGGGSGGGGS (SEQ ID NO: 484), GGGGSGGGGSGGGGS (SEQ ID NO: 30), or GGGGSGGGGSGGGGSGGGGS (SEQ ID NO: 22). The scFv may be arranged in the NT-VH2-VL2-CT or NT-VL2-VH2-CT orientation. NT or nt stands for N-terminus and CT or ct stands for C-terminus of the protein. CH1, CH2, and CH3 are the domains from the IgG Fc region, and CL stands for Constant Light chain, which can be either kappa or lambda family light chains. The other definitions stand for the way they are normally used in the art.

In some embodiments, the VH1 and VL1 domains are derived from the effector molecule and the VH2 and VL2 domains are derived from the targeting moiety. In some embodiments the VH1 and VL1 domains are derived from a targeting moiety and the VH2 and VL2 domains are derived from an effector binding/modulating moiety.

In some embodiments, the VH1 and VL1 domains are derived from an anti-PD-1 antibody, and the VH2 and VL2 domains are derived from an anti-CDH16 antibody. In some embodiments the VH1 and VL1 domains are derived from an anti-CDH16 antibody and the VH2 and VL2 domains are derived from an anti-PD-1 antibody.

In some embodiments, Linker A comprises 1, 2, 3, 4, or 5 GGGGS (SEQ ID NO: 23) repeats. In some embodiments, Linker B comprises 1, 2, 3, 4, or 5 GGGGS (SEQ ID NO: 23) repeats. For the avoidance of doubt, the sequences of Linker A and Linker B, which are used throughout this application, are independent of one another. Therefore, in some embodiments, Linker A and Linker B can be the same or different. In some embodiments, Linker A comprises GGGGS (SEQ ID NO: 23), or two repeats thereof, GGGGSGGGGSGGGGS (SEQ ID NO: 30), or GGGGSGGGGSGGGGSGGGGS (SEQ ID NO: 22). In some embodiments, Linker B comprises GGGGS (SEQ ID NO: 23), or two repeats thereof, GGGGSGGGGS (SEQ ID NO: 484), GGGGSGGGGSGGGGS (SEQ ID NO: 30), or GGGGSGGGGSGGGGSGGGGS (SEQ ID NO: 22).

In some embodiments, the therapeutic compound comprises a light chain and a heavy chain. In some embodiments, the light and heavy chain begin at the N-terminus with the VH domain of a targeting moiety followed by the CH1 domain of a human IgG1, which is fused to a Fc region (e.g., CH2-CH3) of human IgG1. In some embodiments, at the C-terminus of the Fc region is fused to a linker as provided herein, such as but not limited to, GGGGS (SEQ ID NO: 23), or two or three repeats thereof, GGGGSGGGGS (SEQ ID NO: 484), or GGGGSGGGGSGGGGS (SEQ ID NO: 30). The linker can then be fused to an effector binding/modulating moiety, such as any one of the effector moieties provided for herein. The polypeptides can homodimerize because through the heavy chain homodimerization, which results in a therapeutic compound having two effector moieties, such as two anti-PD-1 antibodies. In this orientation, the targeting moiety is an IgG format, there are two Fab arms that each recognize binding partner of the targeting moiety, for example, CDH16 being bound by the CDH16 targeting moiety.

In some embodiments, the targeting moiety is an CDH16 antibody. In some embodiments, the targeting moiety is an OCT2 antibody.

In some embodiments, the antibody is linked to another antibody or therapeutic. In some embodiments, the anti-CDH16 antibody is linked to a PD-1 antibody, an IL-2 mutein, or CD39 as provided herein or that is incorporated by reference.

In some embodiment, the antibody CDH16 antibody, as provided herein, is linked to a IL-2 mutein comprising L118I, N88D, V69A, Q74P, and C125S mutations, and having the following sequence:

(SEQ ID NO: 120)
APTSSSTKKTQLQLEHLLLDLQMILNGINNYKNPKLTRMLTFKFYMPKKA
TELKHLQCLEEELKPLEEALNLAPSKNFHLRPRDLISDINVIVLELKGSE
TTEMCEYADETATIVEFINRWITFSQSIISTLT

In some embodiments, as provided for herein, the anti-CDH16 antibody, is linked directly or indirectly to a PD-1 antibody or binding fragment thereof.

In some embodiments, the PD-1 antibody is selected from the following table:

PD-1 Antibody Table

| Clone | | | | | | | | |
|---|---|---|---|---|---|---|---|---|
| (scFv) | VH Seg | VK Seg | CDR1 | CDR2 | CDR3 | LCDR1 | LCDR2 | LCDR3 |
| PD1AB1 | QVQLVQSGAE | DIQMTQS | GSFTGYY | GWINPN | CARDT | QASHDID | SSLQS | QQANSLPL |
| | VKKPGASVKV | PSSLSAS | MH (SEQ | DGAIHY | VTGDF | KYLN | (SEQ | T (SEQ |
| | SCKASGGSFT | VGDRVTI | ID NO: | A (SEQ | DYW | (SEQ ID | ID | ID NO: |
| | GYYMHWVRQA | TCQASHD | 139) | ID NO: | (SEQ | NO: | NO: | 144) |
| | PGQGLEWMGW | IDKYLNW | | 140) | ID | 142) | 143) | |
| | INPNDGAIHY | YQQKPGK | | | NO: | | | |
| | AQNFQGRVTM | APKLLIY | | | 141) | | | |
| | TRDTSTSTVY | AASSLQS | | | | | | |
| | MELSSLRSED | GVPSRES | | | | | | |
| | TAVYYCARDT | GSGSGTD | | | | | | |
| | VTGDFDYWGQ | FTLTISS | | | | | | |
| | GTLVTVSS | LQPEDFA | | | | | | |
| | (SEQ ID | TYYCQQA | | | | | | |
| | NO: 137) | NSLPLTF | | | | | | |
| | | GGGTKVE | | | | | | |
| | | IK (SEQ | | | | | | |
| | | ID NO: | | | | | | |
| | | 138) | | | | | | |
| PD1AB2 | QVQLVQSGAE | DIQMTQS | GTFSRYA | GWINPN | CAKQG | RASQSIS | STLES | QQSYSTPF |
| | VKKPGASVKV | PSSLSAS | VS (SEQ | SGGTSY | DYGGG | SWLA | (SEQ | T (SEQ |
| | SCKASGGTFS | VGDRVTI | ID NO: | A (SEQ | YYFDY | (SEQ ID | ID | ID NO: |
| | RYAVSWVRQA | TCRASQS | 147) | ID NO: | W | NO: | NO: | 152) |
| | PGQGLEWMGW | ISSWLAW | | 148) | (SEQ | 150) | 151) | |
| | INPNSGGTSY | YQQKPGK | | | ID | | | |
| | AQRFQGRVTM | APKLLIY | | | NO: | | | |
| | TRDTSTSTVY | KTSTLES | | | 149) | | | |
| | MELSSLRSED | GVPSRES | | | | | | |
| | TAVYYCAKQG | GSGSGTD | | | | | | |
| | DYGGGYYFDY | FTLTISS | | | | | | |
| | WGQGTLVTVS | LQPEDFA | | | | | | |
| | S (SEQ ID | TYYCQQS | | | | | | |
| | NO: 145) | YSTPFTF | | | | | | |
| | | GQGTKVE | | | | | | |
| | | IK (SEQ | | | | | | |
| | | ID NO: | | | | | | |
| | | 146) | | | | | | |
| PD1AB3 | QVQLVQSGAE | DIQMTQS | GTFSSYA | GWMNPN | CARVG | RASQSIN | SSLQS | QQSYSTPF |
| | VKKPGASVKV | PSSLSAS | IS (SEQ | SGNTGY | YSYGY | NWLA | (SEQ | T (SEQ |
| | SCKASGGTFS | VGDRVTI | ID NO: | A (SEQ | GMDVW | (SEQ ID | ID | ID NO: |
| | SYAISWVRQA | TCRASQS | 155) | ID NO: | (SEQ | NO: | NO: | 152) |
| | PGQGLEWMGW | INNWLAW | | 156) | ID | 158) | 143) | |
| | MNPNSGNTGY | YQQKPGK | | | NO: | | | |
| | AQKFQGRVTM | APKLLIY | | | 157) | | | |
| | TRDTSTSTVY | AASSLQS | | | | | | |
| | MELSSLRSED | GVPSRES | | | | | | |
| | TAVYYCARVG | GSGSGTD | | | | | | |
| | YSYGYGMDVW | FTLTISS | | | | | | |
| | GQGTTVTVSS | LQPEDFA | | | | | | |
| | (SEQ ID | TYYCQQS | | | | | | |
| | NO: 153) | YSTPFTF | | | | | | |
| | | GPGTKVD | | | | | | |
| | | IK (SEQ | | | | | | |
| | | ID NO: | | | | | | |
| | | 154) | | | | | | |
| PD1AB4 | QVQLVQSGAE | DIQMTQS | YSFTTYY | GIINPS | CASGW | QASRDIK | SSLQS | QQSYSTPP |
| | VKKPGASVKV | PSSLSAS | MH (SEQ | GGSTSY | VYW | NYLA | (SEQ | T (SEQ |
| | SCKASGYSFT | VGDRVTI | ID NO: | A (SEQ | (SEQ | (SEQ ID | ID | ID NO: |
| | TYYMHWVRQA | TCQASRD | 161) | ID NO: | ID | NO: | NO : | 165) |
| | PGQGLEWMGI | IKNYLAW | | 162) | NO: | 164) | 143) | |
| | INPSGGSTSY | YQQKPGK | | | 163) | | | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 159) | YSTPPTF | | | | | | |
| | | GPGTKVD | | | | | | |
| | | IK (SEQ | | | | | | |
| | | ID NO: | | | | | | |
| | | 160) | | | | | | |
| PD1AB5 | EVQLLESGGG | DIQMTQS | FTFSSYA | AAIWSD | CARGL | RASQSIS | STLOS | QQSYSTPL |
| | LVQPGGSLRL | PSSLSAS | MS (SEQ | GSHQYY | GVERG | SWLA | (SEQ | T (SEQ |
| | SCAASGFTFS | VGDRVTI | ID NO: | A (SEQ | LDYW | (SEQ ID | ID | ID NO: |
| | SYAMSWVRQA | TCRASQS | 168) | ID NO: | (SEQ | NO: | NO: | 172) |
| | PGKGLEWVAA | ISSWLAW | | 169) | ID | 150) | 171) | |
| | IWSDGSHQYY | YQQKPGK | | | NO: | | | |
| | ADSVKGRFTI | APKLLIY | | | 170) | | | |
| | SRDNSKNTLY | AASTLQS | | | | | | |
| | LQMNSLRAED | GVPSRES | | | | | | |
| | TAVYYCARGL | GSGSGTD | | | | | | |
| | GVERGLDYWG | FTLTISS | | | | | | |
| | QGTLVTVSS | LQPEDFA | | | | | | |
| | (SEQ ID | TYYCQQS | | | | | | |
| | NO: 166) | YSTPLTF | | | | | | |
| | | GQGTKVE | | | | | | |
| | | IK (SEQ | | | | | | |
| | | ID NO: | | | | | | |
| | | 167) | | | | | | |
| PD1AB6 | EVQLLESGGG | DIQMTQS | FTFSNYP | ALISDD | CARDS | RASQSIN | SNLET | QQSYSTPL |
| | LVQPGGSLRL | PSSLSAS | MH (SEQ | GTNEHY | KFANY | NYLS | (SEQ | T (SEQ |
| | SCAASGFTES | VGDRVTI | ID NO: | A (SEQ | YYYYD | (SEQ ID | ID | ID NO: |
| | NYPMHWVRQA | TCRASQS | 175) | ID NO: | MDVW | NO: | NO: | 172) |
| | PGKGLEWVAL | INNYLSW | | 176) | (SEQ | 178) | 179) | |
| | ISDDGTNEHY | YQQKPGK | | | ID | | | |
| | ADSVKGRFTI | APKLLIY | | | NO: | | | |
| | SRDNSKNTLY | DASNLET | | | 177) | | | |
| | LQMNSLRAED | GVPSRFS | | | | | | |
| | TAVYYCARDS | GSGSGTD | | | | | | |
| | KFANYYYYYD | FTLTISS | | | | | | |
| | MDVWGQGTTV | LQPEDFA | | | | | | |
| | TVSS (SEQ | TYYCQQS | | | | | | |
| | ID NO: | YSTPLTF | | | | | | |
| | 173) | GPGTKVD | | | | | | |
| | | IK (SEQ | | | | | | |
| | | ID NO: | | | | | | |
| | | 174) | | | | | | |
| PD1AB7 | QVQLVQSGAE | DIVMTQS | YSFTGHY | GIINPN | CARGK | RSSQSIL | STRQS | QQYYSIPV |
| | VKKPGASVKV | PDSLAVS | IH (SEQ | GGSTTY | FDFYG | YSSNNRD | (SEQ | T (SEQ |
| | SCKASGYSFT | LGERATI | ID NO: | A (SEQ | DYVTA | YLA | ID | ID NO: |
| | GHYIHWVRQA | NCRSSQS | 182) | ID NO: | FDIW | (SEQ ID | NO: | 187) |
| | PGQGLEWMGI | ILYSSNN | | 183) | (SEQ | NO: | 186) | |
| | INPNGGSTTY | RDYLAWY | | | ID | 185 | | |
| | AQKLQGRVTM | QQKPGQP | | | NO: | | | |
| | TRDTSTSTVY | PKLLIYW | | | 184) | | | |
| | MELSSLRSED | ASTRQSG | | | | | | |
| | TAVYYCARGK | VPDRESG | | | | | | |
| | FDFYGDYVTA | SGSGTDF | | | | | | |
| | FDIWGQGTMV | TLTISSL | | | | | | |
| | TVSS (SEQ | QAEDVAV | | | | | | |
| | ID NO: | YYCQQYY | | | | | | |
| | 180) | SIPVTFG | | | | | | |
| | | GGTKVEI | | | | | | |
| | | K (SEQ | | | | | | |
| | | ID NO: | | | | | | |
| | | 181) | | | | | | |
| PD1AB8 | QVQLVQSGAE | DIQMTQS | YTFSNYD | GWMNPN | CARGA | RASQSIN | SSLQG | QQSYSFPY |
| | VKKPGASVKV | PSSLSAS | MN (SEQ | SGHTGS | FGLHL | NWLA | (SEQ | T (SEQ |
| | SCKASGYTFS | VGDRVTI | ID NO: | A (SEQ | GELSL | (SEQ ID | ID | ID NO: |
| | NYDMNWVRQA | TCRASQS | 190) | ID NO: | HYYGM | NO: | NO: | 194) |
| | PGQGLEWMGW | INNWLAW | | 191) | DVW | 158) | 193) | |
| | MNPNSGHTGS | YQQKPGK | | | (SEQ | | | |
| | APKFQGRVTM | APKLLIY | | | ID | | | |
| | TRDTSTSTVY | AASSLQG | | | NO: | | | |
| | MELSSLRSED | GVPSRFS | | | 192) | | | |
| | TAVYYCARGA | GSGSGTD | | | | | | |
| | FGLHLGELSL | FTLTISS | | | | | | |
| | HYYGMDVWGQ | LQPEDFA | | | | | | |
| | GTTVTVSS | TYYCQQS | | | | | | |
| | (SEQ ID | YSFPYTF | | | | | | |
| | NO: 188) | GQGTKLE | | | | | | |
| | | IK (SEQ | | | | | | |
| | | ID NO: | | | | | | |
| | | 189) | | | | | | |
| PD1AB9 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ |
| | SCKASGYTFT | VGDRVTI | ID NO: | A (SEQ | TW | (SEQ ID | ID | ID NO: |
| | GYYMHWVRQA | TCRASQS | 197) | ID NO: | (SEQ | NO: | NO: | 201) |
| | PGQGLEWMGK | ISRWLAW | | 198) | ID | 200) | 143) | |
| | IVPMFDAANY | YQQKPGK | | | NO: | | | |
| | APKFQGRVTI | APKLLIY | | | 199) | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTLTISS | | | | | | |
| | TLVTVSS | LQPEDFA | | | | | | |
| | (SEQ ID | TYYCQQA | | | | | | |
| | NO: 195) | NSFPVTF | | | | | | |
| | | GGGTKVE | | | | | | |
| | | IK (SEQ | | | | | | |
| | | ID NO: | | | | | | |
| | | 196) | | | | | | |
| PD1AB10 | QVQLVQSGAE | DIQMTQS | YTFTGYY | GIINPS | CAKTA | RASQSIN | SSLQS | QQGYSVPL |
| | VKKPGASVKV | PSSLSAS | MH (SEQ | GGSTSY | GYDWL | SWLA | (SEQ | S (SEQ |
| | SCKASGYTFT | VGDRVTI | ID NO: | A (SEQ | PSGLG | (SEQ ID | ID | ID NO: |
| | GYYMHWVRQA | TCRASQS | 197) | ID NO: | MDVW | NO: | NO: | 206) |
| | PGQGLEWMGI | INSWLAW | | 162) | (SEQ | 205) | 143) | |
| | INPSGGSTSY | YQQKPGK | | | ID | | | |
| | AQKFQGRVTM | APKLLIY | | | NO: | | | |
| | TRDTSTSTVY | YASSLQS | | | 204) | | | |
| | MELSSLRSED | GVPSRFS | | | | | | |
| | TAVYYCAKTA | GSGSGTD | | | | | | |
| | GYDWLPSGLG | FTLTISS | | | | | | |
| | MDVWGQGTTV | LQPEDFA | | | | | | |
| | TVSS (SEQ | TYYCQQG | | | | | | |
| | ID NO: | YSVPLSF | | | | | | |
| | 202) | GQGTKLE | | | | | | |
| | | IK (SEQ | | | | | | |
| | | ID NO: | | | | | | |
| | | 203) | | | | | | |
| PD1AB11 | QVQLVQSGAE | DIQMTQS | YTFSNYG | GGIIPI | CARWR | RASQGIS | SNLET | QQSYSTPL |
| | VKKPGSSVKV | PSSLSAS | IT (SEQ | FGSTAS | SDAFD | NWLA | (SEQ | T (SEQ |
| | SCKASGYTFS | VGDRVTI | ID NO: | YA | IW | (SEQ ID | ID | ID NO: |
| | NYGITWVRQA | TCRASQG | 209) | (SEQ | (SEQ | NO: | NO: | 172) |
| | PGQGLEWMGG | ISNWLAW | | ID NO: | ID | 212) | 179) | |
| | IIPIFGSTAS | YQQKPGK | | 210) | NO: | | | |
| | YAQKFQGRVT | APKLLIY | | | 211) | | | |
| | ITADESTSTA | DASNLET | | | | | | |
| | YMELSSLRSE | GVPSRFS | | | | | | |
| | DTAVYYCARW | GSGSGTD | | | | | | |
| | RSDAFDIWGQ | FTLTISS | | | | | | |
| | GTMVTVSS | LQPEDFA | | | | | | |
| | (SEQ ID | TYYCQQS | | | | | | |
| | NO: 207) | YSTPLTF | | | | | | |
| | | GGGTKVE | | | | | | |
| | | IK (SEQ | | | | | | |
| | | ID NO: | | | | | | |
| | | 208) | | | | | | |
| PD1AB12 | QVQLVQSGAE | DIQMTQS | GTFSTYA | GWINPN | CARVN | RASQGIR | STLNS | QQSYSTPF |
| | VKKPGASVKV | PSSLSAS | IS (SEQ | SGGTNY | YDFYY | NDLG | (SEQ | T (SEQ |
| | SCKASGGTFS | VGDRVTI | ID NO: | A (SEQ | GMDVW | (SEQ ID | ID | ID NO: |
| | TYAISWVRQA | TCRASQG | 215) | ID NO: | (SEQ | NO: | NO: | 152) |
| | PGQGLEWMGW | IRNDLGW | | 216) | ID | 218) | 219) | |
| | INPNSGGTNY | YQQKPGK | | | NO: | | | |
| | AQKFQGRVTM | APKLLIY | | | 217) | | | |
| | TRDTSTSIVY | RASTLNS | | | | | | |
| | MELSSLRSED | GVPSRFS | | | | | | |
| | TAVYYCARVN | GSGSGTD | | | | | | |
| | YDFYYGMDVW | FTLTISS | | | | | | |
| | GQGTTVTVSS | LQPEDFA | | | | | | |
| | (SEQ ID | TYYCQQS | | | | | | |
| | NO: 213) | YSTPFTF | | | | | | |
| | | GPGTKVD | | | | | | |
| | | IK (SEQ | | | | | | |
| | | ID NO: | | | | | | |
| | | 214) | | | | | | |
| PD1AB13 | QVQLVQSGAE | DIQMTQS | GTFSTYA | GWINPN | CARVN | RASQGIR | STLNS | QQSYSTPF |
| | VKKPGASVKV | PSSLSAS | IS (SEQ | SGGTNY | YDFYY | NDLG | (SEQ | T (SEQ |
| | SCKASGGTFS | VGDRVTI | ID NO: | A (SEQ | GMDVW | (SEQ ID | ID | ID NO: |
| | TYAISWVRQA | TCRASQG | 215) | ID NO: | (SEQ | NO: | NO: | 152) |
| | PGQGLEWMGW | IRNDLGW | | 216) | ID | 218) | 219) | |
| | INPNSGGTNY | YQQKPGK | | | NO: | | | |
| | AQKFQGRVTM | APKLLIY | | | 217) | | | |
| | TRDTSTSTVY | RASTLNS | | | | | | |
| | MELSSLRSED | GVPSRES | | | | | | |
| | TAVYYCARVN | GSGSGTD | | | | | | |
| | YDFYYGMDVW | FTLTISS | | | | | | |
| | GQGTTVTVSS | LQPEDFA | | | | | | |
| | (SEQ ID | TYYCQQS | | | | | | |
| | NO: 220) | YSTPFTF | | | | | | |
| | | GPGTKVD | | | | | | |
| | | IK (SEQ | | | | | | |
| | | ID NO: | | | | | | |
| | | 214) | | | | | | |
| PD1AB14 | EVQLLESGGG | DIQMTQS | FSFSSYD | SGISGS | CASPY | RASQDIA | SSVQT | QQSYTTPY |
| | LVQPGGSLRL | PSSLSAS | MS (SEQ | GSSTYY | GMGYM | NYLA | (SEQ | T (SEQ |
| | SCAASGFSFS | VGDRVTI | ID NO: | A (SEQ | DVW | (SEQ ID | ID | ID NO: |
| | SYDMSWVRQA | TCRASQD | 223) | ID NO: | (SEQ | NO : | NO: | 228) |
| | PGKGLEWVSG | IANYLAW | | 224; | ID | 226) | 227) | |
| | ISGSGSSTYY | YQQKPGK | | | NO: | | | |
| | ADSVKGRFTI | APKLLIY | | | 225) | | | |
| | SRDNSKNTLY | GASSVQT | | | | | | |
| | LQMNSLRAED | GVPSRFS | | | | | | |
| | TAVYYCASPY | GSGSGTD | | | | | | |
| | GMGYMDVWGK | FTLTISS | | | | | | |
| | GTTVTVSS | LQPEDFA | | | | | | |
| | (SEQ ID | TYYCQQS | | | | | | |
| | NO: 221) | YTTPYTF | | | | | | |
| | | GQGTRLE | | | | | | |
| | | IK (SEQ | | | | | | |
| | | ID NO: | | | | | | |
| | | 222) | | | | | | |
| PD1AB15 | QVQLVQSGAE | DIQMTQS | GSFNNYA | GWINPN | CARVS | QASQDIS | SNLQS | QQSYSTPF |
| | VKKPGASVKV | PSSLSAS | IS (SEQ | TGGTSY | YGVGY | RYLN | (SEQ | T (SEQ |
| | SCKASGGSFN | VGDRVTI | ID NO: | A (SEQ | YMDVW | (SEQ ID | ID | ID NO: |
| | NYAISWVRQA | TCQASQD | 231) | ID NO: | (SEQ | NO: | NO: | 152) |
| | PGQGLEWMGW | ISRYLNW | | 232) | ID | 234) | 235) | |
| | INPNTGGTSY | YQQKPGK | | | NO: | | | |
| | AQKFQGRVTM | APKLLIY | | | 233) | | | |
| | TRDTSTSTVY | AASNLQS | | | | | | |
| | MELSSLRSED | GVPSRES | | | | | | |
| | TAVYYCARVS | GSGSGTD | | | | | | |
| | YGVGYYMDVW | FTLTISS | | | | | | |
| | GKGTTVTVSS | LQPEDFA | | | | | | |
| | (SEQ ID | TYYCQQS | | | | | | |
| | NO: 229) | YSTPFTF | | | | | | |
| | | GPGTKVD | | | | | | |
| | | IK (SEQ | | | | | | |
| | | ID NO: | | | | | | |
| | | 230) | | | | | | |
| PD1AB16 | QVQLVQSGAE | DIQMTQS | GSFNNYA | GWINPN | CARVS | QASQDIS | SNLQS | QQSYSTPF |
| | VKKPGASVKV | PSSLSAS | IS (SEQ | TGGTSY | YGVGY | RYLN | (SEQ | T (SEQ |
| | SCKASGGSFN | VGDRVTI | ID NO: | A (SEQ | YMDVW | (SEQ ID | ID | ID NO: |
| | NYAISWVRQA | TCQASQD | 231) | ID NO: | (SEQ | NO: | NO: | 152) |
| | PGQGLEWMGW | ISRYLNW | | 232) | ID | 234) | 235) | |
| | INPNTGGTSY | YQQKPGK | | | NO: | | | |
| | AQKFQGRVTM | APKLLIY | | | 233) | | | |
| | TRDTSTSTVY | AASNLQS | | | | | | |
| | MELSSLRSED | GVPSRES | | | | | | |
| | TAVYYCARVS | GSGSGTD | | | | | | |
| | YGVGYYMDVW | STLTISS | | | | | | |
| | GKGTTVTVSS | LQPEDFA | | | | | | |
| | (SEQ I | TYYCQQS | | | | | | |
| | NO: 229) | YSTPFTF | | | | | | |
| | | GPGTKVD | | | | | | |
| | | IK (SEQ | | | | | | |
| | | ID NO: | | | | | | |
| | | 236) | | | | | | |
| PD1AB17 | QVQLVQSGAE | DIQMTQS | YTFTDDY | GWMNTN | CARGG | RASQGVG | SSLQS | QQAYSFPW |
| | VKKPGASVKV | PSSLSAS | IH (SEQ | SGNTGY | SYSSG | NALG | (SEQ | T (SEQ |
| | SCKASGYTFT | VGDRVTI | ID NO: | A (SEQ | WYGRL | (SEQ ID | ID | ID NO: |
| | DDYIHWVRQA | TCRASQG | 239) | ID NO: | DYYYG | NO: | NO: | 243) |
| | PGQGLEWMGW | VGNALGW | | 240) | MDVW | 242) | 143) | |
| | MNTNSGNTGY | YQQKPGK | | | (SEQ | | | |
| | AQKFQGRVTM | APKLLIY | | | ID | | | |
| | TRDTSTSTVY | AASSLQS | | | NO: | | | |
| | MELSSLRSED | GVPSRFS | | | 241) | | | |
| | TAVYYCARGG | GSGSGTD | | | | | | |
| | SYSSGWYGRL | FTLTISS | | | | | | |
| | DYYYGMDVWG | LQPEDFA | | | | | | |
| | QGTTVTVSS | TYYCQQA | | | | | | |
| | (SEQ ID | YSFPWTF | | | | | | |
| | NO: 237) | GQGTKLE | | | | | | |
| | | IK (SEQ | | | | | | |
| | | ID NO: | | | | | | |
| | | 238) | | | | | | |
| PD1AB18 | QVQLVQSGAE | DIQMTQS | YTFTDYA | GWLNPN | CAAGL | RASQSIN | SSLES | QQSYSIPI |
| | VKKPGASVKV | PSSLSAS | MH (SEQ | SGNTGY | FIW | RWLA | (SEQ | T (SEQ |
| | SCKASGYTFT | VGDRVTI | ID NO: | A (SEQ | (SEQ | (SEQ | ID | ID NO: |
| | DYAMHWVRQA | TCRASQS | 246) | ID NO: | ID | ID | NO: | 251) |
| | PGQGLEWMGW | INRWLAW | | 247) | NO: | NO: | 250) | |
| | LNPNSGNTGY | YQQKPGK | | | 248) | 249) | | |
| | APKFQGRVTM | APKLLIY | | | | | | |
| | TRDTSTSTVY | DASSLES | | | | | | |
| | MELSSLRSED | GVPSRES | | | | | | |
| | TAVYYCAAGL | GSGSGTD | | | | | | |
| | FIWGQGTMVT | FTLTISS | | | | | | |
| | VSS (SEQ | LQPEDFA | | | | | | |
| | ID NO: | TYYCQQS | | | | | | |
| | 244) | YSIPITF | | | | | | |
| | | GQGTRLE | | | | | | |
| | | IK (SEQ | | | | | | |
| | | ID NO: | | | | | | |
| | | 245) | | | | | | |
| PD1AB19 | QVQLVQSGAE | DIVMTQS | GTFSSYA | GGIIPG | CTTEY | RSSQSLL | SNRAP | MQALQTPL |
| | VKKPGSSVKV | PLSLPVT | IS (SEQ | FGSPNY | CSSTS | HSNGYNY | (SEQ | T (SEQ |
| | SCKASGGTFS | PGEPASI | ID NO: | A (SEQ | CSDYW | LD (SEQ | ID | ID NO: |
| | SYAISWVRQA | SCRSSQS | 155) | ID NO: | (SEQ | ID NO: | NO: | 258) |
| | PGQGLEWMGG | LLHSNGY | | 254) | ID | 256) | 257) | |
| | IIPGFGSPNY | NYLDWYL | | | NO: | | | |
| | APNFQGRVTI | QKPGQSP | | | 255) | | | |
| | TADESTSTAY | QLLIYQG | | | | | | |
| | MELSSLRSED | SNRAPGV | | | | | | |
| | TAVYYCTTEY | PDRFSGS | | | | | | |
| | CSSTSCSDYW | GSGTDFT | | | | | | |
| | GQGTLVTVSS | LKISRVE | | | | | | |
| | (SEQ ID | AEDVGVY | | | | | | |
| | NO: 252) | YCMQALQ | | | | | | |
| | | TPLTFGQ | | | | | | |
| | | GTKVEIK | | | | | | |
| | | (SEQ ID | | | | | | |
| | | NO : | | | | | | |
| | | 253) | | | | | | |
| PD1AB20 | QVQLVQSGAE | DIQMTQS | YTFSDHY | GTINPS | CAADN | RASQSIS | STLQS | QQSHSLPL |
| | VKKPGASVKV | PSSLSAS | MH (SEQ | GGRTSY | GHASG | NWVA | (SEQ | T (SEQ |
| | SCKASGYTFS | VGDRVTI | ID NO: | A (SEQ | WLYYY | (SEQ ID | ID | ID NO: |
| | DHYMHWVRQA | TCRASQS | 261) | ID NO: | GMDVW | NO: | NO: | 265) |
| | PGQGLEWMGT | ISNWVAW | | 262) | (SEQ | 264) | 171) | |
| | INPSGGRTSY | YQQKPGK | | | ID | | | |
| | AQKFQGRVTM | APKLLIY | | | NO: | | | |
| | TRDTSTSTVY | RASTLQS | | | 263) | | | |
| | MELSSLRSED | GVPSRES | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 259) | GPGTKVD | | | | | | |
| | | IK (SEQ | | | | | | |
| | | ID NO: | | | | | | |
| | | 260) | | | | | | |
| PD1AB21 | EVQLLESGGG | DIQMTQS | FTFSSYA | SGISGG | CASEY | RASQSIS | SSLQS | QQYRNFPY |
| | LVQPGGSLRL | PSSLSAS | MS (SEQ | GGTTYY | YGMDV | GWLA | (SEQ | T (SEQ |
| | SCAASGFTFS | VGDRVTI | ID NO: | A (SEQ | W | (SEQ ID | ID | ID NO: |
| | SYAMSWVRQA | TCRASQS | 168) | ID NO: | (SEQ | NO: | NO: | 271) |
| | PGKGLEWVSG | ISGWLAW | | 268) | ID | 270) | 143) | |
| | ISGGGGTTYY | YQQKPGK | | | NO: | | | |
| | ADSVKGRFTI | APKLLIY | | | 269) | | | |
| | SRDNSKNTLY | AASSLQS | | | | | | |
| | LQMNSLRAED | GVPSRES | | | | | | |
| | TAVYYCASEY | GSGSGTD | | | | | | |
| | YGMDVWGQGT | FTLTISS | | | | | | |
| | TVTVSS | LQPEDFA | | | | | | |
| | (SEQ ID | TYYCQQY | | | | | | |
| | NO: 266) | RNFPYTF | | | | | | |
| | | GQGTKLE | | | | | | |
| | | IK (SEQ | | | | | | |
| | | ID NO: | | | | | | |
| | | 267) | | | | | | |
| PD1AB22 | QVQLVQSGAE | EIVMTQS | YTFSGYY | GVINPS | CAEGF | RASQGVG | STRAT | QQYYTTPI |
| | VKKPGASVKV | PATLSVS | MH (SEQ | GGSTSY | DYW | RSLA | (SEQ | T (SEQ |
| | SCKASGYTFS | PGERATL | ID NO: | A (SEQ | (SEQ | (SEQ ID | ID | ID NO: |
| | GYYMHWVRQA | SCRASQG | 274) | ID NO: | ID | NO: | NO: | 279) |
| | PGQGLEWMGV | VGRSLAW | | 275) | NO: | 277) | 278) | |
| | INPSGGSTSY | YQQKPGQ | | | 276) | | | |
| | AQKFQGRVTM | APRLLIY | | | | | | |
| | TRDTSTSTVY | GASTRAT | | | | | | |
| | MELSSLRSED | GIPARES | | | | | | |
| | TAVYYCAEGE | GSGSGTE | | | | | | |
| | DYWGQGTLVT | FTLTISS | | | | | | |
| | VSS (SEQ | LQSEDFA | | | | | | |
| | ID NO: | VYYCQQY | | | | | | |
| | 272) | YTTPITE | | | | | | |
| | | GQGTRLE | | | | | | |
| | | IK (SEQ | | | | | | |
| | | ID NO: | | | | | | |
| | | 273) | | | | | | |
| PD1AB23 | QVQLVQSGAE | DIQMTQS | GTFSNYA | GWMNPN | CARVN | QASQDIS | STLKS | QQADNLPF |
| | VKKPGASVKV | PSSLSAS | IS (SEQ | SGNTGY | YYYYY | NYLN | (SEQ | T (SEQ |
| | SCKASGGTFS | VGDRVTI | ID NO : | A (SEQ | GMDVW | (SEQ ID | ID | ID NO: |
| | NYAISWVRQA | TCQASQD | 282) | ID NO: | (SEQ | NO: | NO: | 286) |
| | PGQGLEWMGW | ISNYLNW | | 156) | ID | 284) | 285) | |
| | MNPNSGNTGY | YQQKPGK | | | NO: | | | |
| | AQKFQGRVTM | APKLLIY | | | 283) | | | |
| | TRDTSTSTVY | KASTLKS | | | | | | |
| | MELSSLRSED | GVPSRFS | | | | | | |
| | TAVYYCARVN | GSGSGTD | | | | | | |
| | YYYYYGMDVW | FTLTISS | | | | | | |
| | GQGTTVTVSS | LQPEDFA | | | | | | |
| | (SEQ ID | TYYCQQA | | | | | | |
| | NO: 280) | DNLPFTF | | | | | | |
| | | GPGTKVD | | | | | | |
| | | IK (SEQ | | | | | | |
| | | ID NO: | | | | | | |
| | | 281) | | | | | | |
| PD1AB24 | QVQLVQSGAE | DIQMTQS | YTFTNYY | GIINPS | CARDW | QASRDIS | SSLQS | QQANSFPP |
| | VKKPGASVKV | PSSLSAS | MH (SEQ | GGSTSY | GWDYY | NYLN | (SEQ | T (SEQ |
| | SCKASGYTFT | VGDRVTI | ID NO: | A (SEQ | YYGMD | (SEQ ID | ID | ID NO: |
| | NYYMHWVRQA | TCQASRD | 289) | ID NO: | VW | NO: | NO: | 292) |
| | PGQGLEWMGI | ISNYLNW | | 162) | (SEQ | 291) | 143) | |
| | INPSGGSTSY | YQQKPGK | | | ID | | | |
| | AQRFQGRVTM | APKLLIY | | | NO: | | | |
| | TRDTSTSTVY | AASSLQS | | | 290) | | | |
| | MELSSLRSED | GVPSRFS | | | | | | |
| | TAVYYCARDW | GSGSGTD | | | | | | |
| | GWDYYYYGMD | FTLTISS | | | | | | |
| | VWGQGTTVTV | LQPEDFA | | | | | | |
| | SS (SEQ ID | TYYCQQA | | | | | | |
| | NO: 287) | NSFPPTF | | | | | | |
| | | GQGTKLE | | | | | | |
| | | IK (SEQ | | | | | | |
| | | ID NO: | | | | | | |
| | | 288) | | | | | | |
| PD1AB25 | EVQLLESGGG | DIVMTQS | FTFSNSD | SGITIS | CARGR | KSSQSVL | STRES | QQYYTTPP |
| | LVQPGGSLRL | PDSLAVS | MS (SEQ | GGSTYY | GGSGW | YSPNNKN | (SEQ | T (SEQ |
| | SCAASGFTES | LGERATI | ID NO: | A (SEQ | LDYW | YLA | ID | ID NO: |
| | NSDMSWVRQA | NCKSSQS | 295) | ID NO: | (SEQ | (SEQ ID | NO: | 300) |
| | PGKGLEWVSG | VLYSPNN | | 296) | ID | NO: | 299) | |
| | ITISGGSTYY | KNYLAWY | | | NO: | 298) | | |
| | ADSVRGRFTI | QQKPGQP | | | 297) | | | |
| | SRDNSKNTLY | PKLLIYW | | | | | | |
| | LQMSSLRAED | ASTRESG | | | | | | |
| | TAVYYCARGR | VPDRESG | | | | | | |
| | GGSGWLDYWG | SGSGTDE | | | | | | |
| | QGTLVTVSS | TLTISSL | | | | | | |
| | (SEQ ID | QAEDVAV | | | | | | |
| | NO: 293) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 294) | | | | | | |
| PD1AB26 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ |
| | SCKASGYTFT | VGDRVTI | ID NO: | A (SEQ | TW | (SEQ ID | ID | ID NO: |
| | GYYMHWVRQA | TCRASQS | 197) | ID NO: | (SEQ | NO: | NO: | 201) |
| | PGQGLEWMGK | ISRWLAW | | 198) | ID | 200) | 143) | |
| | IVPMFDAANY | YQQKPGK | | | NO: | | | |
| | APKFQGRVTI | APKLLIY | | | 199) | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTLTISS | | | | | | |
| | TLVTVSS | LQPEDFA | | | | | | |
| | (SEQ ID | TYYCQQA | | | | | | |
| | NO: 195) | NSFPVTF | | | | | | |
| | | GGGTKVD | | | | | | |
| | | IK (SEQ | | | | | | |
| | | ID NO: | | | | | | |
| | | 301) | | | | | | |
| PD1AB27 | QVQLVQSGAE | DIQMTQS | YTFTGYY | GKIVPM | CARGP | RASQSIS | SSLOS | QQANSFPV |
| | VKKPGSSVKV | PSSLSAS | MH (SEQ | FDAANY | KWELD | RWLA | (SEQ | T (SEQ |
| | SCKASGYTFT | VGDRVTI | ID NO: | A (SEQ | TW | (SEQ ID | ID | ID NO: |
| | GYYMHWVRQA | TCRASQS | 197) | ID NO: | (SEQ | NO: | NO: | 201) |
| | PGQGLEWMGK | ISRWLAW | | 198) | ID | 200) | 143) | |
| | IVPMFDAANY | YQQKPGK | | | NO: | | | |
| | APKFQGRVTI | APKLLIY | | | 199) | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTFTISS | | | | | | |
| | TLVTVSS | LQPEDFA | | | | | | |
| | (SEQ ID | TYYCQQA | | | | | | |
| | NO: 195) | NSFPVTF | | | | | | |
| | | GGGTKVD | | | | | | |
| | | IK (SEQ | | | | | | |
| | | ID NO: | | | | | | |
| | | 302) | | | | | | |
| PD1AB28 | QVQLVQSGAE | DIQMTQS | GDFSNYF | GWINPH | CARGG | RASQSIS | STLOS | QQSYSTPF |
| | VKKPGSSVKV | PSSLSAS | VS (SEQ | NGDTMY | YSYGY | TWLA | (SEQ | T (SEQ |
| | SCKASGGDFS | VGDRVTI | ID NO: | A (SEQ | TFDIW | (SEQ ID | ID | ID NO: |
| | NYFVSWVRQA | TCRASQS | 305) | ID NO: | (SEQ | NO: | NO: | 152) |
| | PGQGLEWMGW | ISTWLAW | | 306) | ID | 308) | 171) | |
| | INPHNGDTMY | YQQKPGK | | | NO: | | | |
| | AQKFQGRVTI | APKLLIY | | | 307) | | | |
| | TADESTSTAY | AASTLQS | | | | | | |
| | MELSSLRSED | GVPSRES | | | | | | |
| | TAVYYCARGG | GSGSGTD | | | | | | |
| | YSYGYTFDIW | FTLTISS | | | | | | |
| | GQGTMVTVSS | LQPEDFA | | | | | | |
| | (SEQ ID | TYYCQQS | | | | | | |
| | NO: 303) | YSTPFTF | | | | | | |
| | | GGGTKVE | | | | | | |
| | | IK (SEQ | | | | | | |
| | | ID NO: | | | | | | |
| | | 304) | | | | | | |
| PD1AB29 | EVQLLESGGG | DIVMTQS | FTFSNSD | SGITIS | CARGR | KSSQSVL | STRES | QQYYITPP |
| | LVQPGGSLRL | PDSLAVS | MS (SEQ | GGSTYY | GGSGW | YSPNNKN | (SEQ | T (SEQ |
| | SCAASGFTES | LGERATI | ID NO: | A (SEQ | LDYW | YLA | ID | ID NO: |
| | NSDMSWVRQA | NCKSSQS | 295) | ID NO: | (SEQ | (SEQ ID | NO: | 311) |
| | PGKGLEWVSG | VLYSPNN | | 296) | ID | NO: | 299) | |
| | ITISGGSTYY | KNYLAWY | | | NO: | 298) | | |
| | ADSVKGRFTI | QQKPGQP | | | 297) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDE | | | | | | |
| | QGTLVTVSS | TLTISSL | | | | | | |
| | (SEQ ID | QAEDVAV | | | | | | |
| | NO: 309) | YYCQQYY | | | | | | |
| | | ITPPTFG | | | | | | |
| | | QGTRLEI | | | | | | |
| | | K (SEQ | | | | | | |
| | | ID NO: | | | | | | |
| | | 310) | | | | | | |
| PD1AB30 | EVQLLESGGG | DIVMTQS | FTFSNSD | SGITIS | CARGR | KSSQSVL | STRES | QQYYTTPP |
| | LVQPGGSLRL | PDSLAVS | MS (SEQ | GGSTYY | GGSGW | YSPNNKN | (SEQ | T (SEQ |
| | SCAASGFTES | LGERATI | ID NO: | A (SEQ | LDYW | YLA | ID | ID NO: |
| | NSDMSWVRQA | NCKSSQS | 295) | ID NO: | (SEQ | (SEQ ID | NO: | 300) |
| | PGKGLEWVSG | VLYSPNN | | 296) | ID | NO: | 299) | |
| | ITISGGSTYY | KNYLAWY | | | NO: | 298) | | |
| | ADSVKGRFTI | QQKPGQP | | | 297) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID | QAEDVAV | | | | | | |
| | NO: 309) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 294) | | | | | | |
| PD1AB31 | QVQLVQSGAE | EIVMTQS | HTFTDYY | GIINPS | CASGW | RASQSVS | SSRAT | QQYTTSPI |
| | VKKPGSSVKV | PATLSVS | MH (SEQ | GGSTSY | TDW | SYLA | (SEQ | T (SEQ |
| | SCKASGHTFT | PGERATL | ID NO: | A (SEQ | (SEQ | (SEQ ID | ID | ID NO: |
| | DYYMHWVRQA | SCRASQS | 314) | ID NO: | ID | NO: | NO: | 318) |
| | PGQGLEWMGI | VSSYLAW | | 162) | NO: | 316) | 317) | |
| | INPSGGSTSY | YQQKPGQ | | | 315) | | | |
| | AQKFQGRVTI | APRLLIY | | | | | | |
| | TADESTSTAY | GTSSRAT | | | | | | |
| | MELSSLRSED | GIPARFS | | | | | | |
| | TAVYYCASGW | GSGSGTE | | | | | | |
| | TDWGQGTLVT | FTLTISS | | | | | | |
| | VSS (SEQ | LQSEDFA | | | | | | |
| | ID NO: | VYYCQQY | | | | | | |
| | 312) | TTSPITF | | | | | | |
| | | GQGTRLE | | | | | | |
| | | IKR | | | | | | |
| | | (SEQ ID | | | | | | |
| | | NO: | | | | | | |
| | | 313) | | | | | | |
| PD1AB32 | QVQLVQSGAE | DIQMTQS | YTFTDYY | GGIFPV | CARDH | QASQDIS | KDLHP | QESFSTLT |
| | VKKPGASVKV | PSSLSAS | MH (SEQ | FGSSTY | GSGLD | NYLN | (SEQ | (SEQ ID |
| | SCKASGYTFT | VGDRVTI | ID NO: | A (SEQ | VW | (SEQ ID | ID | NO: 325) |
| | DYYMHWVRQA | TCQASQD | 321) | ID NO: | (SEQ | NO: | NO : | |
| | PGQGLEWMGG | ISNYLNW | | 322) | ID | 284) | 324) | |
| | IFPVFGSSTY | YQQKPGK | | | NO: | | | |
| | AQKFQGRVTM | APKLLIY | | | 323) | | | |
| | TRDTSTSTVY | DAKDLHP | | | | | | |
| | MELSSLRSED | GVPSRFS | | | | | | |
| | TAVYYCARDH | GSGSGTD | | | | | | |
| | GSGLDVWGQG | FTLTISS | | | | | | |
| | TTVTVSS | LQPEDFA | | | | | | |
| | (SEQ ID | TYYCQES | | | | | | |
| | NO: 319) | FSTLTFG | | | | | | |
| | | QGTKVEI | | | | | | |
| | | KR (SEQ | | | | | | |
| | | ID NO: | | | | | | |
| | | 320) | | | | | | |
| PD1AB33 | QVQLVQSGAE | DIQMTQS | YSFTTYY | GIIAPS | CASGW | QASRDIK | SSLQS | QQSYSTPP |
| | VKKPGASVKV | PSSLSAS | MH (SEQ | GGSTSY | VYW | NYLA | (SEQ | T (SEQ |
| | SCKASGYSFT | VGDRVTI | ID NO: | A (SEQ | (SEQ | (SEQ ID | ID | ID NO: |
| | TYYMHWVRQA | TCQASRD | 161) | ID NO: | ID | NO: | NO: | 165) |
| | PGQGLEWMGI | IKNYLAW | | 327) | NO: | 164) | 143) | |
| | IAPSGGSTSY | YQQKPGK | | | 163) | | | |
| | AQKFQGRVTM | APKLLIY | | | | | | |
| | TRDTSTSTVY | AASSLQS | | | | | | |
| | MELSSLRSED | GVPSRES | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 326) | YSTPPTF | | | | | | |
| | | GPGTKVD | | | | | | |
| | | IK (SEQ | | | | | | |
| | | ID NO: | | | | | | |
| | | 160) | | | | | | |
| PD1AB34 | QVQLVQSGAE | DIQMTQS | YSFTTYY | GIIAPS | CASGW | QASRDIK | SSLQS | QQSYSTPP |
| | VKKPGASVKV | PSSLSAS | MH (SEQ | GGSTSY | VYW | NYLA | (SEQ | T (SEQ |
| | SCKASGYSFT | VGDRVTI | ID NO: | A (SEQ | (SEQ | (SEQ ID | ID | ID NO: |
| | TYYMHWVRQA | TCQASRD | 161) | ID NO: | ID | NO: | NO: | 165) |
| | PGQGLEWMGI | IKNYLAW | | 327) | NO: | 164) | 143) | |
| | IGPSGGSTSY | YQQKPGK | | | 163) | | | |
| | AQKFQGRVTM | APKLLIY | | | | | | |
| | TRDTSTSTVY | AASSLQS | | | | | | |
| | MELSSLRSED | GVPSRES | | | | | | |
| | TAVYYCASGW | GSGSGTD | | | | | | |
| | VYWGQGTLVT | FTLTISS | | | | | | |
| | VSS (SEQ | LQPEDFA | | | | | | |
| | ID NO: | TYYCQQS | | | | | | |
| | 328) | YSTPPTF | | | | | | |
| | | GPGTKVD | | | | | | |
| | | IK (SEQ | | | | | | |
| | | ID NO: | | | | | | |
| | | 160) | | | | | | |
| PD1AB35 | QVQLVQSGAE | DIQMTQS | YTFSDHY | GTIAPS | CAADN | RASQSIS | STLQS | QQSHSLPL |
| | VKKPGASVKV | PSSLSAS | MH (SEQ | GGRTSY | GHASG | NWVA | (SEQ | T (SEQ |
| | SCKASGYTFS | VGDRVTI | ID NO: | A (SEQ | WLYYY | (SEQ ID | ID | ID NO: |
| | DHYMHWVRQA | TCRASQS | 261) | ID NO: | GMDVW | NO: | NO: | 265) |
| | PGQGLEWMGT | ISNWVAW | | 330) | (SEQ | 264) | 171) | |
| | IAPSGGRTSY | YQQKPGK | | | ID | | | |
| | AQKFQGRVTM | APKLLIY | | | NO: | | | |
| | TRDTSTSTVY | RASTLQS | | | 263) | | | |
| | MELSSLRSED | GVPSRES | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 329) | GPGTKVD | | | | | | |
| | | IK (SEQ | | | | | | |
| | | ID NO: | | | | | | |
| | | 260) | | | | | | |
| PD1AB36 | QVQLVQSGAE | DIQMTQS | YTFSDHY | GTIAPS | CAADN | RASQSIS | STLQS | QQSHSLPL |
| | VKKPGASVKV | PSSLSAS | MH (SEQ | GGRTSY | GHASG | NWVA | (SEQ | T (SEQ |
| | SCKASGYTES | VGDRVTI | ID NO: | A (SEQ | WLYYY | (SEQ ID | ID | ID NO: |
| | DHYMHWVRQA | TCRASQS | 261) | ID NO: | GMDVW | NO: | NO: | 265) |
| | PGQGLEWMGT | ISNWVAW | | 330) | (SEQ | 264) | 171) | |
| | IGPSGGRTSY | YQQKPGK | | | ID | | | |
| | AQKFQGRVTM | APKLLIY | | | NO: | | | |
| | TRDTSTSTVY | RASTLQS | | | 263) | | | |
| | MELSSLRSED | GVPSRES | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 331) | GPGTKVD | | | | | | |
| | | IK (SEQ | | | | | | |
| | | ID NO: | | | | | | |
| | | 260) | | | | | | |
| PD1AB37 | QVQLVQSGAE | DIQMTQS | TYYMH | IINPSG | ASGWV | QASRDIK | AASSL | QQSYSTPP |
| | VKKPGASVKV | PSSLSAS | (SEQ ID | GSTSYA | Y | NYLA | QS | T (SEQ |
| | SCKASGYSFT | VGDRVTI | NO: | QKFQG | (SEQ | (SEQ ID | (SEQ | ID NO: |
| | TYYMHWVRQA | TCQASRD | 332) | (SEQ | ID | NO: | ID | 165) |
| | PGQGLEWMGI | IKNYLAW | | ID NO: | NO: | 164) | NO: | |
| | INPSGGSTSY | YQQKPGK | | 333) | 334) | | 335) | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVI | FTLTISS | | | | | | |
| | VSS (SEQ | LQPEDFA | | | | | | |
| | ID NO: | TYYCQQS | | | | | | |
| | 159) | YSTPPTF | | | | | | |
| | | GPGTKVD | | | | | | |
| | | IK (SEQ | | | | | | |
| | | ID NO: | | | | | | |
| | | 160) | | | | | | |
| PD1AB38 | EVQLLESGGG | DIVMTQS | SNSDMS | GITISG | ARGRG | KSSQSVL | WASTR | QQYYTTPP |
| | LVQPGGSLRL | PDSLAVS | (SEQ ID | GSTYYA | GSGWL | YSPNNKN | ES | T (SEQ |
| | SCAASGFTFS | LGERATI | NO: | DSVK | DY | YLA | (SEQ | ID NO: |
| | NSDMSWVRQA | NCKSSQS | 336) | (SEQ | (SEQ | (SEQ ID | ID | 300) |
| | PGKGLEWVSG | VLYSPNN | | ID NO: | ID | NO: | NO: | |
| | ITISGGSTYY | KNYLAWY | | 337) | NO: | 298) | 339) | |
| | ADSVKGRFTI | QQKPGQP | | | 338) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID | QAEDVAV | | | | | | |
| | NO: 309) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 294) | | | | | | |

In some embodiments, the PD-1 antibody comprises a sequence as shown in PD-1 Antibody Table. In some embodiments, the antibody is in a scFV format as illustrated in the PD-1 Antibody Table. In some embodiments, the antibody comprises a CDR1 from any one of clones of the PD-1 Antibody Table, a CDR2 from any one of clones of the PD-1 Antibody Table, and a CDR3 from any one of clones of the PD-1 Antibody Table. In some embodiments, the antibody comprises a LCDR1 from any one of clones of the PD-1 Antibody Table, a LCDR2 from any one of clones of the PD-1 Antibody Table, and a LCDR3 from any one of clones of the PD-1 Antibody Table. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the PD-1 antibody has a VH region selected from any one of clones of the PD-1 Antibody Table and a VL region selected from any one of clones as set forth in the PD-1 Antibody Table.

In some embodiments, as provided for herein, the PD-1 antibody, or binding fragment thereof, is linked directly or indirectly to an anti-CHD16 antibody.

In some embodiments, as provided for herein, the anti-CDH16 antibody, or binding fragment thereof, is linked directly or indirectly to a IL-2 mutein or binding fragment thereof. The IL-2 mutein can be any mutein as provided for herein or other IL-2 muteins known to one of skill in the art.

In some embodiments, as provided for herein, the anti-CDH16 antibody, or binding fragment thereof, is linked directly or indirectly to a CD39 molecule.

In some embodiments, the CD39 molecule is linked to the anti-CDH16 antibody in the N- to C-terminus direction. In some embodiments, the C-terminus of an anti-CDH16 antibody scFv is linked via a G/S linked to the N-terminus of a CD39 molecule.

In some embodiments, the CD39 molecule is linked to the anti-CDH16 antibody in the C- to N-terminus direction. In some embodiments, the C-terminus of CD39 molecule is linked via a G/S linker to the N-terminus of an Fc molecule further linked at the N-terminus of the Fc molecule via a G/S linker to the N-terminus of an anti-CDH16 antibody scFv.

In some embodiments, the anti-CDH16 antibody linked to the CD39 molecule comprises a heterodimeric molecule, further comprising a bivalent anti-CDH16 antibody and monovalent human CD39 molecules.

In some embodiments, the anti-CDH16 antibody linked to the CD39 molecule comprises a heterodimeric molecule, further comprising a monovalent anti-CDH16 antibody or an anti-OCT2 antibody and monovalent human CD39 molecules.

In some embodiments, the C-terminus of the CD39 is linked via a G/S or A/E linker to the N-terminus of the Fc. In some embodiments, the Fc-CD39, has the sequence of:

(CD39AB1, SEQ ID NO: 445)
DKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVESCSVMHE
ALHNHYTQKSLSLSPGGGGGSNVKYGIVLDAGSSHTSLYIYKWPAEKENDTGVVHQVECRVKGPGISKEV
QKVNEIGIYLTDCMERAREVIPRSQHQETPVYLGATAGMRLLRMESEELADRVLDVVERSLSNYPEDFQG
ARIITGQEEGAYGWITINYLLGKFSQKTRWFSIVPYETNNQETFGALDLGGASTQVTFVPQNQTIHSFLC
YGKDQALWQKLAKDIQVASNEILRDPCFHPGYKKVVNVSDLYKTPCTKRFEMTLPFQQFEIQGIGNYQQC
HQSILELFNTSYCPYSQCAFNGIFLPPLQGDFGAFSAFYFVMKELNLTSEKVSQEKVTEMMKKFCAQPWE
EIKTSYAGVKEKYLSEYCFSGTYILSLLLQGYHFTADSWEHIHFIGKIQGSDAGWTLGYMLNLINMIPAE
QPLSTPLSHSTYV
(CD39AB2, SEQ ID NO: 446)
DKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHE
ALHNHYTQKSLSLSPGGGGGSNVKYGIVLDAGSSHTSLYIYKWPAEKENDTGVVHQVECRVKGPGISKFV
QKVNEIGIYLTDCMERAREVIPRSQHQETPVYLGATAGMRLLRMESEELADRVLDVVERSLSNYPEDEQG
ARIITGQEEGAYGWITINYLLGKFSQKTRWESIVPYETNNQETFGALDLGGASTQVTFVPQNQTIHSFLC
YGKDQALWQKLAKDIQVASNEILRDPCFHPGYKKVVNVSDLYKTPCTKRSEMTLPFQQFEIQGIGNYQQC
HQSILELENTSYCPYSQCAFNGIFLPPLQGDFGAFSAFYFVMKELNLTSEKVSQEKVTEMMKKFCAQPWE
EIKTSYAGVKEKYLSEYCFSGTYILSLLLQGYHFTADSWEHIHFIGKIQGSDAGWTLGYMLNLTNMIPAE
QPLSTPLSHSTYV
(CD39AB3, SEQ ID NO: 447)
DKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHE
ALHNHYTQKSLSLSPGGGGGSNVKYGIVLDAGSSHTSLYIYKWPAEKENDTGVVHQVECRVKGPGISKEV
QKVNEIGIYLTDCMERAREVIPRSQHQETPVYLGATAGMRLLRMESEELADRVLDVVERSLSNYPEDEQG
ARIITGQEEGAYGWITINYLLGKFSQKTRWESIVPYETNNQETFGALDLGGASTQVTFVPQNQTIHSFLC
YGKDQALWQKLAKDIQVASNEILRDPCFHPGYKKVVNVSDLYKTPCTKRFEMTEPFQQFEIQGIGNYQQC
HQSILELENTSYCPYSQCAFNGIFLPPLQGDFGAFSAFYFVMKELNLTSEKVSQEKVTEMMKKFCAQPWE
EIKTSYAGVKEKYLSEYCFSGTYILSLLLQGYHFTADSWEHIHFIGKIQGSDAGWTLGYMLNLTNMIPAE
QPLSTPLSHSTYV
(CD39AB4, SEQ ID NO: 448)
DKTHTCPPCPAPEAAGAPSVFLFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHE
ALHNHYTQKSLSLSPGGGGGSNVKYGIVLDAGSSHTSLYIYKWPAEKENDTGVVHQVECRVKGPGISKEV
QKVNEIGIYLTDCMERAREVIPRSQHQETPVYLGATAGMRLLRMESEELADRVLDVVERSLSNYPEDFQG
ARIITGQEEGAYGWITINYLLGKFSQKTRWFSIVPYETNNQETFGALDLGGASTQVTFVPQNQTIHSFLC
YGKDQALWQKLAKDIQVASNEILRDPCFHPGYKKVVNVSDLYKTPCTKRFEMTLPFQQTEIQGIGNYQQC
HQSILELFNTSYCPYSQCAFNGIFLPPLQGDFGAFSAFYFVMKELNLTSEKVSQEKVTEMMKKFCAQPWE
EIKTSYAGVKEKYLSEYCFSGTYILSLLLQGYHFTADSWEHIHFIGKIQGSDAGWTLGYMLNLTNMIPAE
QPLSTPLSHSTYV
(CD39AB5, SEQ ID NO: 449)
DKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHE
ALHNHYTQKSLSLSPGGGGGSNVKYGIVLDAGSSHTSLYIYKWPAEKENDTGVVHQVECRVKGPGISKFV
QKVNEIGIYLTDCMERAREVIPRSQHQETPVYLGATAGMRLLRMESEELADRVLDVVERSLSNYPEDEQG
ARIITGQEEGAYGWITINYLLGKFSQKTRWFSIVPYETNNQETFGALDLGGASTQVTFVPQNQTIHSFLC
YGKDQALWQKLAKDIQVASNEILRDPCFHPGYKKVVNVSDLYKTPCTKRFEMTLPFQQFEIQGIGNYQQC
HQSILELFNTSYCPYSQCAFNGIFLPPLQGDFGAFSAFYFVMKELNLTSEKVSQEKVTEMMKKFCAQPWE
EIKTSYAGVKEKYLSEYCRSGTYILSLLLQGYHFTADSWEHIHFIGKIQGSDAGWTLGYMLNLTNMIPAE
QPLSTPLSHSTYV
(CD39AB6, SEQ ID NO: 450)
DKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHE
ALHNHYTQKSLSLSPGGGGGSNVKYGIVLDAGSSHTSLYIYKWPAEKENDTGVVHQVECRVKGPGISKEV
QKVNEIGIYLTDCMERAREVIPRSQHQETPVYLGATAGMRLLRMESEELADRVLDVVERSLSNYPEDEQG
ARIITGQEEGAYGWITINYLLGKFSQKTRWFSIVPYETNNQETFGALDLGGASTQVTFVPQNQTIHSFLC
YGKDQALWQKLAKDIQVASNEILRDPCFHPGYKKVVNVSDLYKTPCTKRFEMTLPFQQFEIQGIGNYQQC
HQSILELFNTSYCPYSQCAFNGIFLPPLQGDFGAFSAFYFVMKELNLTSEKVSQEKVTEMMKKFCAQPWE
EIKTSYAGVKEKYLSEYCFSGTYISSLLLQGYHFTADSWEHIHFIGKIQGSDAGWTLGYMLNLTNMIPAE
QPLSTPLSHSTYV;
or
(CD39AB7, SEQ ID NO: 451)
DKTHTCPPCPAPEAAGAPSVELFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVESCSVMHE
ALHNHYTQKSLSLSPGGGGGSNVKYGIVLDAGSSHTSLYIYKWPAEKENDTGVVHQVECRVKGPGISKEV
QKVNEIGIYLTDCMERAREVIPRSQHQETPVYLGATAGMRLLRMESEELADRVLDVVERSLSNYPFDEQG
ARIITGQEEGAYGWITINYLLGKFSQKTRWFSIVPYETNNQETFGALDLGGASTQVTFVPQNQTIHSFLC
YGKDQALWQKLAKDIQVASNEILRDPCFHPGYKKVVNVSDLYKTPCTKRFEMTLPFQQFEIQGIGNYQQC
HQSILELENTSYCPYSQCAFNGIFLPPLQGDFGAFSAFYFVMKELNLTSEKVSQEKVTEMMKKFCAQPWE
EIKTSYAGVKEKYLSEYCFSGTYILSLLSQGYHFTADSWEHIHFIGKIQGSDAGWTLGYMLNLINMIPAE
QPLSTPLSHSTYV

In some embodiments, the N-terminus of the CD39 is linked via a G/S or A/E linker to the C-terminus of the Fc. In some embodiments, the CD39-Fc, has the sequence of:

(CD39AB8, SEQ ID NO: 452)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWQKLAKDIQVASNE
GIFLPPLQGDFGAFSAFYFVMKELNLTSEKVSQEKVTEMMKKFCAQPWEEIKTSYAGVKEKYLSEYCESG
TYILSLLLQGYHFTADSWEHIHFIGKIQGSDAGWTLGYMLNLINMIPAEQPLSTPLSHSTYVGGGGSGGG
GSGGGGSGGGGSDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB9, SEQ ID NO: 453)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWOKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKELNLTSEKVSQEKVTEMMKKFCAQPWEEIKTSYAGVKEKYLSEYCFSG
TYILSLLLQGYHFTADSWEHIHFIGKIQGSDAGWTLGYMLNLINMIPAEQPLSTPLSHSTYVGGGSEGGG
SEGGGSEGGGSEDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB10, SEQ ID NO: 454)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWQKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKELNLTSEKVSQEKVTEMMKKFCAQPWEEIKTSYAGVKEKYLSEYCESG
TYILSLLLQGYHFTADSWEHIHFIGKIQGSDAGWTLGYMLNLTNMIPAEQPLSTPLSHSTYVAEEEKAEE
EKAEEEKAEEEKDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB11, SEQ ID NO: 455)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWQKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKFLNLTSEKVSQEKVTEMMKKFCAQPWEEIKTSYAGVKEKYLSEYCESG
TYILSLLLQGYHFTADSWEHIHFIGKIQGSDAGWTLGYMLNLTNMIPAEQPLSTPLSHSTYVGGGGSGGG
GSGGGGSGGGGSDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB12, SEQ ID NO: 456)
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWQKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKELNLTSEKVSQEKVTEMMKKFCAQPWEEIKTSYAGVKEKYLSEYCFSG
TYILSLLLQGYHFTADSWEHIHFIGKIQGSDAGWTLGYMLNLTNMIPAEQPLSTPLSHSTYVGGGSEGGG
SEGGGSEGGGSEDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB13, SEQ ID NO: 457)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWOKLAKDIQVASNE
ILRDPCFHPGYKKVVNVSDLYKTPCTKRFEMTLPFQQFEIQGIGNYQQCHQSILELENTSYCPYSQCAFN
GIFLPPLQGDFGAFSAFYFVMKELNLTSEKVSQEKVTEMMKKFCAQPWEEIKTSYAGVKEKYLSEYCESG
TYILSLLLQGYHFTADSWEHIHFIGKIQGSDAGWTLGYMLNLINMIPAEQPLSTPLSHSTYVAEEEKAEE
EKAEEEKAEEEKDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB14, SEQ ID NO: 458)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPEDFQGARIITGQEEGAYGWITINYLL
GKFSQKTRWFSIVPYETNNQETFGALDLGGASTQVTFVPQNQTIHSFLCYGKDQALWQKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKFLNLTSEKVSQEKVTEMMKKFCAQPWEEIKTSYAGVKEKYLSEYCESG
TYILSLLLQGYHFTADSWEHIHFIGKIQGSDAGWTLGYMLNLTNMIPAEQPLSTPLSHSTYVGGGGSGGG
GSGGGGSGGGGSDKTHTCPPCPAPEAAGAPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG
(CD39AB15, SEQ ID NO: 459)
NVKYGIVLDAGSSHTSLYIYKWPAEKENDTGVVHQVECRVKGPGISKEVQKVNEIGIYLTDCMERAREVI
PRSQHQETPVYLGATAGMRLLRMESEELADRVLDVVERSLSNYPFDFQGARIITGQEEGAYGWITINYLL
GKFSQKTRWFSIVPYETNNQETFGALDLGGASTQVTFVPQNQTIHSFLCYGKDQALWQKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKELNLTSEKVSQEKVTEMMKKFCAQPWEEIKTSYAGVKEKYLSEYCESG
TYILSLLLQGYHFTADSWEHIHFIGKIQGSDAGWTLGYMLNLINMIPAEQPLSTPLSHSTYVGGGSEGGG
SEGGGSEGGGSEDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG;
or
(CD39AB16, SEQ ID NO: 460)
NVKYGIVLDAGSSHTSLYIYKWPAEKENDTGVVHQVECRVKGPGISKFVQKVNEIGIYLTDCMERAREVI
PRSQHQETPVYLGATAGMRLLRMESEELADRVLDVVERSLSNYPFDFQGARIITGQEEGAYGWITINYLL
GKFSQKTRWFSIVPYETNNQETFGALDLGGASTQVTFVPQNQTIHSFLCYGKDQALWQKLAKDIQVASNE
ILRDPCFHPGYKKVVNVSDLYKTPCTKRFEMTLPFQQFEIQGIGNYQQCHQSILELENTSYCPYSQCAEN
GIFLPPLQGDFGAFSAFYFVMKELNLTSEKVSQEKVTEMMKKFCAQPWEEIKTSYAGVKEKYLSEYCESG
TYILSLLLQGYHFTADSWEHIHFIGKIQGSDAGWTLGYMLNLTNMIPAEQPLSTPLSHSTYVAEEEKAEE
EKAEEEKAEEEKDKTHTCPPCPAPEAAGAPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPG

The molecules comprising an anti-CDH16 antibody and a PD-1 antibody, an IL-2 mutein, or a CD39 can be various formats as described herein. For example, they can be in the following formats:

### PD-1 ML-N Format:

Heavy Chain: NT-[VH_PD-1]-[CH1-CH2-CH3]-[LinkerA]-[anti-CDH16 antibodyscFv]-CT
Light Chain: NT-[VK_PD-1]-[CK]-CT

### PD-1 ML-N(2) Format:

Heavy Chain: NT-[VH_PD-1]-[CH1-CH2-CH3]-[LinkerC]-[anti-CDH16 antibodyscFab]-CT
Light Chain: NT-[VK_PD-1]-[CK]-CT

### PD-1 ML-C Format:

Heavy Chain: NT-[VH_anti-CDH16 antibody]-[CH1-CH2-CH3]-[LinkerA]-[PD-1 scFv]-CT
Light Chain: NT-[VK_anti-CDH16 antibody]-[CK]-CT

### PD-1 CL-N Format:

Light Chain: NT-[VH_PD-1]-[CK]-[LinkerD]-[anti-CDH16 antibodyscFab]-CT
Heavy Chain: NT-[VK_PD-1]-[CH1-CH2-CH3]-CT

### PD-1 IgG Format:

Heavy Chain: NT-[VH_PD-1]-[CH1-CH2-CH3]
Light Chain: NT-[VK_PD-1]-[CK]-CT

### IL-2 ML-N Format:

Heavy Chain: NT-[VH_anti-CDH16 antibody]-[CH1-CH2-CH3]-[LinkerA]-[IL-2 mutein]-CT
Light Chain: NT-[VK_anti-CDH16 antibody]-[CL]-CT

### IL-2 CL-N Format:

Light Chain: NT-[VK_anti-CDH16 antibody]-[CL]-[LinkerC/D]-[IL-2 mutein]-CT
Heavy Chain: NT-[VH_anti-CDH16 antibody]-[CH1-CH2-CH3]-CT

### CD39 ML-N Format:

Heavy Chain: NT-[VH_anti-CDH16 antibody]-[CH1-CH2-CH3]-[LinkerA/B/C]-[CD39]-CT
Light Chain: NT-[VK_anti-CDH16 antibody]-[CL]-CT

### CD39 ML-N(2) Format:

Heavy Chain: NT-[CD39]-[LinkerA/B/C]-[VH_anti-CDH16 antibody]-[CH1-CH2-CH3]-CT
Light Chain: NT-[VK_anti-CDH16 antibody]-[CL]-CT

### CD39 CL-N Format:

Light Chain: NT-[VK_anti-CDH16 antibody]-[CL]-[LinkerA/B/C]-[CD39]-CT
Heavy Chain: NT-[VH_anti-CDH16 antibody]-[CH1-CH2-CH3]-CT

### CD39 CL-N(2) Format:

Light Chain: NT-[CD39]-[LinkerA/B/C/D]-[VK_anti-CDH16 antibody]-[CL]-CT
Heavy Chain: NT-[VH_anti-CDH16 antibody]-[CH1-CH2-CH3]-CT

### CD39 ML-N(3) Format:

Heavy Chain 1: NT-[VH_anti-CDH16 antibody]-[CH1-CH2-CH3]-[LinkerA/B/C/D]-[CD39]-CT
Heavy Chain 2: NT-[VH_anti-CDH16 antibody]-[CH1-CH2-CH3]-CT
Light Chain: NT-[VK_anti-CDH16 antibody]-[CL]-CT

### CD39 ML-N(4) Format:

Heavy Chain 1: NT-[VH_anti-CDH16 antibody]-[CH1-CH2-CH3]-CT
Heavy Chain 2: NT-[IgG1Fc]-[LinkerA/B/C/D]-[CD39]-CT
Light Chain: NT-[VK_anti-CDH16 antibody]-[CL]-CT

### CD39 ML-N(5) Format:

Heavy Chain 1: NT-[VH_anti-CDH16 antibody]-[CH1]-[IgG2H]-[CD39]-CT
Light Chain: NT-[VK_anti-CDH16 antibody]-[CL]-CT

The abbreviations used above are as follows:

| Component | Description |
|---|---|
| NT | N-terminus |
| CT | C-terminus |
| VH_PD-1 | VH domain of PD-1 antibody |
| | as provided herein. |
| VK_PD-1 | VK domain of PD-1 antibody |
| | as provided herein. |
| PD-1scFv | PD-1 antibody in scFv |
| | comprising the VH and VK |
| | domain. |
| VH_anti-CDH16 antibody | VH domain of- anti-CDH16 |
| | antibody Ab as provided |
| | herein. |
| VK_anti-CDH16 antibody | VK domain of- anti- CDH16 |
| | antibody Ab as provided |
| | herein. This can also be |
| | substituted with a VL |
| | sequences as provided herein. |
| anti-CDH16 antibody scFv | anti- CDH16 antibody scFV |
| | Ab as provided herein. |
| anti-CDH16 antibody scFab | anti- CDH16 antibody scFab |
| | Ab as provided herein. |
| VH_anti-CDH16 antibody_BM1 | Rat anti-mouse anti- CDH16 |
| | antibody placeholder VH |
| | domain |
| VK_anti-CDH16 antibody_BM1 | Rat anti-mouse anti- CDH16 |
| | antibody placeholder VK |
| | domain |
| v antibody scFv_BM1 | Rat anti-mouse anti- CDH16 |
| | antibody placeholder scFv |
| VH_PD-1_BM1 | Anti-human PD-1 agonist |
| | placeholder VH domain |
| VK_PD-1_BM1 | Anti-human PD-1 agonist |
| | placeholder VK domain |
| CH1-CH2-CH3 | Human IgG1 Constant Heavy |
| | 1 (CH1), Constant Heavy 2 |
| | (CH2), and Constant Heavy 3 |
| | (CH3) domains |
| IgG1 Fc | Human IgG1 Fc |
| IgG2H | Human IgG2 Hinger region |
| CK | Human constant kappa |
| | domain |
| CL | Constant lambda domain |
| IL-2_Mutein | IL-2 moiety such as those |
| | provided herein. |
| CD39 | CD39 effector domains such |
| | as those provided herein |
| Linker_A | G/S, or G/S/E, or A/E/K |
| | linker (5 amino acid length) |
| Linker_B | G/S, or G/S/E, or A/E/K |
| | linker (10 amino acid length) |
| Linker_C | G/S, or G/S/E, or A/E/K |
| | linker (15 amino acid length) |
| Linker_D | G/S, or G/S/E, or A/E/K |
| | linker (20 amino acid length) |

The sequence of CH1-CH2-CH3 can be, for example,

(SEQ ID NO: 44)
ASTKGPSVFPLAPSSKSTSGGTAALGCLVKDYFPEPVTVSWNSGALTSGVHTFPAVLOSSGLYSLSSVVTVPSSSLG
TQTYICNVNHKPSNTKVDKKVEPKSCDKTHTCPPCPAPEAAGAPSVELFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPG;
or
(KIH mutations; SEQ ID NO: 461)
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGAPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPCREEMTKNQVSLSCAVKGFYPSDIAVEWESNGQPENNYKTTPPVLDSDGSFFLVSKLTVDKSRWQQGNVFSC
SVMHEALHNHYTQKSLSLSPG;
or
(KIH mutations; SEQ ID NO: 462)
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGAPSVELFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
CTLPPSREEMTKNQVSLWCLVKGFYPSDIAVEWESNGQPENNYKTTPPVLDSDGSFFLYSKLTVDKSRWQQGNVESC
SVMHEALHNHYTOKSLSLSPG;
or
(KIH mutations; SEQ ID NO: 463)
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGAPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVLDSDGSFFLVSKLTVDKSRWQQGNVFSC
SVMHEALHNHYTQKSLSLSPG;
or
(KIH mutations; SEQ ID NO: 464)
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPEAAGAPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPCREEMTKNQVSLWCLVKGFYPSDIAVEWESNGQPENNYKTTPPVLDSDGSFFLYSKLTVDKSRWQQGNVFSC
SVMHEALHNHYTQKSLSLSPG

The sequence of CK/CL can be, for example.

(SEQ ID NO: 45)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The sequence of IgG2 hinge can be, for example, EPKSCCVECPPCPAPPAAAGA (SEQ ID NO: 465).

In some embodiments, if the therapeutic compound comprises a Fc portion, the Fc domain, (portion) bears mutations to render the Fc region “effectorless” that is unable to bind FcRs. The mutations that render Fc regions effectorless are known. In some embodiments, the mutations in the Fc region, which is according to the known numbering system, are selected from the group consisting of: K322A, L234A, L235A, G237A, L234F, L235E, N297, P331S, or any combination thereof. In some embodiments, the Fc mutations comprises a mutation at L234 and/or L235 and/or G237. In some embodiments, the Fc mutations comprise L234A and/or L235A mutations, which can be referred to as LALA mutations. In some embodiments, the Fc mutations comprise L234A, L235A, and G237A mutations.

In some embodiments, the Fc portion has a sequence of:

(SEQ ID NO: 466)
DKTHTCPPCPAPEAAGAPSVFLFPPKPKDTLMISRTPEVTCVVVDVSHED
PEVKENWYVDGVEVHNAKTKPREEQYNSTYRVVSVLTVLHQDWLNGKEYK
CKVSNKALPAPIEKTISKAKGQPREPQVYTLPPCREEMTKNQVSLSCAVK
GFYPSDIAVEWESNGQPENNYKTTPPVLDSDGSFFLVSKLTVDKSRWQQG
NVFSCSVMHEALHNHYTQKSLSLSPG;
or
(SEQ ID NO: 467)
DKTHTCPPCPAPEAAGAPSVELFPPKPKDTLMISRTPEVTCVVVDVSHED
PEVKENWYVDGVEVHNAKTKPREEQYNSTYRVVSVLTVLHQDWLNGKEYK
CKVSNKALPAPIEKTISKAKGQPREPQVCTLPPSREEMTKNQVSLWCLVK
GFYPSDIAVEWESNGQPENNYKTTPPVLDSDGSFFLYSKLTVDKSRWQQG
NVFSCSVMHEALHNHYTOKSLSLSPG;
or
(SEQ ID NO: 468)
DKTHTCPPCPAPEAAGAPSVELFPPKPKDTLMISRTPEVTCVVVDVSHED
PEVKENWYVDGVEVHNAKTKPREEQYNSTYRVVSVLTVLHQDWLNGKEYK
CKVSNKALPAPIEKTISKAKGQPREPQVYTLPPCREEMTKNQVSLWCLVK
GFYPSDIAVEWESNGQPENNYKTTPPVLDSDGSFFLYSKLTVDKSRWQQG
NVFSCSVMHEALHNHYTQKSLSLSPG;
or
(SEQ ID NO: 469)
DKTHTCPPCPAPEAAGAPSVFLFPPKPKDTLMISRTPEVTCVVVDVSHED
PEVKENWYVDGVEVHNAKTKPREEQYNSTYRVVSVLTVLHQDWLNGKEYK
CKVSNKALPAPIEKTISKAKGQPREPQVCTLPPSREEMTKNQVSLSCAVK
GFYPSDIAVEWESNGQPENNYKTTPPVLDSDGSFFLVSKLTVDKSRWQQG
NVFSCSVMHEALHNHYTQKSLSLSPG.

Disclosed herein are Linker Region polypeptides, therapeutic peptides, and nucleic acids encoding the polypeptides (e.g., therapeutic compounds), vectors comprising the nucleic acid sequences, and cells comprising the nucleic acids or vectors.

Therapeutic compounds can comprise a plurality of specific targeting moieties. In some embodiments, the therapeutic compound comprises a plurality one specific targeting moiety, a plurality of copies of a donor specific targeting moiety or a plurality of tissue specific targeting moieties. In some embodiments, a therapeutic compound comprises a first and a second donor specific targeting moiety, e.g., a first donor specific targeting moiety specific for a first donor target and a second donor specific targeting moiety specific for a second donor target, e.g., wherein the first and second target are found on the same donor tissue. In some embodiments, the therapeutic compound comprises e.g., a first specific targeting moiety for a tissue specific target and a second specific targeting moiety for a second target, e.g., wherein the first and second target are found on the same or different target tissue.

Polypeptides Derived from Reference, e.g., Human Polypeptides

In some embodiments, a component of a therapeutic molecule is derived from or based on a reference molecule, e.g., in the case of a therapeutic molecule for use in humans, from a naturally occurring human polypeptide. E.g., in some embodiments, all or a part of a CD39 molecule, a specific targeting moiety, a target ligand binding molecule, or a tissue specific targeting moiety, can be based on or derived from a naturally occurring human polypeptide. E.g., a PD-L1 molecule can be based on or derived from a human PD-L1 sequence.

In some embodiments, a therapeutic compound component, e.g., a PD-L1 molecule:
a) comprises all or a portion of, e.g., an active portion of, a naturally occurring form of the human polypeptide;
b) comprises all or a portion of, e.g., an active portion of, a human polypeptide having a sequence appearing in a database, e.g., GenBank database, on Jan. 11, 2017, a naturally occurring form of the human polypeptide that is not associated with a disease state;
c) comprises a human polypeptide having a sequence that differs by no more than 1, 2, 3, 4, 5, 10, 20, or 30 amino acid residues from a sequence of a) or b);
d) comprises a human polypeptide having a sequence that differs by no more than 1, 2, 3, 4, 5 10, 20, or 30% its amino acids residues from a sequence of a) or b);
e) comprises a human polypeptide having a sequence that does not differ substantially from a sequence of a) or b); or
f) comprises a human polypeptide having a sequence of c), d), or e) that does not differ substantially in biological activity, e.g., ability to enhance or inhibit an immune response, from a human polypeptide having the sequence of a) or b).

### Pharmaceutical Compositions and Kits

In another aspect, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible.

The carrier can be suitable for intravenous, intramuscular, subcutaneous, parenteral, rectal, local, ophthalmic, topical, spinal or epidermal administration (e.g., by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating, and coloring agents can be used. The carriers can be used in pharmaceutical compositions comprising the therapeutic compounds provided for herein.

The compositions and compounds of the embodiments provided herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

In some embodiments, embodiments provided herein also include, but are not limited to:
1. A polypeptide comprising a kidney targeting moiety that binds to a target kidney cell and an effector binding/modulating moiety, wherein the effector binding/modulating moiety is a PD-1 agonist, a CD39 Effector Domain, or an IL-2 mutein polypeptide (IL-2 mutein).
2. The polypeptide of embodiment 1, wherein the kidney targeting moiety comprises an antibody that binds to a target protein on the surface of the kidney cell.
3. The polypeptide of embodiment 2, wherein the antibody is an antibody that binds to a CDH16 or an OCT2 protein.
4. The polypeptide of any one of embodiments 1-3, wherein the effector binding/modulating moiety binds to a receptor expressed by an immune cell.
5. The polypeptide of embodiment 4, wherein the effector binding/modulating moiety is a PD-1 agonist, a CD39 Effector Domain, or an IL-2 mutein.
6. The polypeptide of embodiment 4, wherein the immune cell contributes to an unwanted immune response.
7. The polypeptide of any one of embodiments 4 or 6, wherein the immune cell causes a disease pathology.
8. The polypeptide of any one of embodiments 1-7, wherein the targeting moiety comprises an anti-CDH16 antibody or an anti-OCT 2 antibody.
9. The polypeptide of embodiment 1, wherein the polypeptide has the formula from N-terminus to C-terminus:
   R1-Linker Region A-R2; or
   R3-Linker Region B-R4,
wherein,
   R1, R2, R3, and R4, each independently comprises the effector binding/modulating moiety, the targeting moiety, or is absent, provided that at least one of R1 and R2 is not absent, and at least one of R3 and R4 is not absent.
10. The polypeptide of embodiment 9, wherein the polypeptide having the formula of R1-Linker Region A-R2 and the polypeptide having the formula of R3-Linker Region B-R4 interact with one another to form a polypeptide complex.
11. The polypeptide of embodiment 9, wherein the polypeptide having the formula of R1-Linker Region A-R2 and the polypeptide having the formula of R3-Linker Region B-R4 do not interact with one another to form a polypeptide complex.
12. The polypeptide of any one of embodiments 9-11, wherein each of Linker Region A and Linker Region B comprises an Fc region.
13. The polypeptide of any one of embodiments 9-12, wherein the Linker Region A and Linker Region B each comprise a linker.
14. The polypeptide of embodiment 13, wherein the linker is absent.
15. The polypeptide of embodiment 13, wherein the linker comprises a Fc region.
16. The polypeptide of embodiment 13, wherein the linker comprises a glycine/serine linker.
17. The polypeptide of embodiment 16, wherein the linker is GGGGSGGGGSGGGGSGGGGS (SEQ ID NO: 22), GGGGSGGGGSGGGGS (SEQ ID NO: 30), GGGGSGGGGS (SEQ ID NO: 484), or GGGGS (SEQ ID NO: 23).
18. The polypeptide of any one of embodiments 9-11, wherein one of R1 and R2 is an IL-2 mutein and one of R1 and R2 is an anti-CDH16 antibody or an anti-OCT2 antibody.
19. The polypeptide of any one of embodiments 9-11 or 18, wherein R1 is an IL-2 mutein and R2 is anti-CDH16 antibody or an anti-OCT2 antibody.
20. The polypeptide of any one of embodiments 9-11 or 18, wherein R1 is an anti-CDH16 antibody or an anti-OCT2 antibody and R2 is an IL-2 mutein.
21 The polypeptide of any one of embodiments 9-11, wherein one of R3 and R4 is an IL-2 mutein and one of R3 and R4 is an anti-CDH16 antibody or an anti-OCT2 antibody.
22. The polypeptide of any one of embodiments 9-11 or 21, wherein R3 is an IL-2 mutein and R4 is an anti-CDH16 antibody or an anti-OCT2 antibody.
23. The polypeptide of any one of embodiments 9-11 or 21, wherein R3 is anti-CDH16 antibody or an anti-OCT2 antibody and one R4 is an IL-2 mutein.
24. The polypeptide of any one of embodiments 9-11, wherein one of R1 and R2 is an anti-PD-1 antibody and one of R1 and R2 is an anti-CDH16 antibody or an anti-OCT2 antibody.
25. The polypeptide of any one of embodiments 9-11 or 24, wherein R1 is an anti-PD-1 antibody and R2 is anti-CDH16 antibody or an anti-OCT2 antibody.
26. The polypeptide of any one of embodiments 9-11 or 24, wherein R1 is an anti-CDH16 antibody or an anti-OCT2 antibody and R2 is an anti-PD-1 antibody.
27. The polypeptide of any one of embodiments 9-11, wherein one of R3 and R4 is an anti-PD-1 antibody and one of R3 and R4 is an anti-CDH16 antibody or an anti-OCT2 antibody.
28. The polypeptide of any one of embodiments 9-11 or 27, wherein R3 is an anti-PD-1 antibody and R4 is an anti-CDH16 antibody or an anti-OCT2 antibody.
29. The polypeptide of any one of embodiments 9-11 or 27, wherein R3 is anti-CDH16 antibody or an anti-OCT2 antibody and one R4 is an anti-PD-1 antibody.
30 The polypeptide of any one of embodiments 9-11, wherein one of R1 and R2 is a CD39 Effector Domain and one of R1 and R2 is an anti-CDH16 antibody or an anti-OCT2 antibody.
31. The polypeptide of any one of embodiments 9-11 or 30, wherein R1 is a CD39 Effector Domain and R2 is anti-CDH16 antibody or an anti-OCT2 antibody.
32. The polypeptide of any one of embodiments 9-11 or 30, wherein R1 is an anti-CDH16 antibody or an anti-OCT2 antibody and R2 is a CD39 Effector Domain.
33. The polypeptide of any one of embodiments 9-11, wherein one of R3 and R4 is a CD39 Effector Domain and one of R3 and R4 is an anti-CDH16 antibody or an anti-OCT2 antibody.
34. The polypeptide of any one of embodiments 9-11 or 33, wherein R3 is a CD39 Effector Domain and R4 is an anti-CDH16 antibody or an anti-OCT2 antibody.
35 The polypeptide of any one of embodiments 9-11 or 33, wherein R3 is anti-CDH16 antibody or an anti-OCT2 antibody and one R4 is a CD39 Effector Domain.
36. The polypeptide of any one of embodiments 1, 5, or 18-23, wherein the IL-2 mutein comprises a IL-2 sequence of SEQ ID NO: 6, and wherein the sequence comprises a mutation at a position that corresponds to position 53, 56, 80, or 118 of SEQ ID NO: 6.
37. The polypeptide of any one of embodiments 1, 5, or 18-23, wherein the IL-2 mutein comprises the sequence of SEQ ID NO: 60, wherein at least one of X₁, X₂, X₃, and X₄ is I and the remainder are L or I.
38. The polypeptide of embodiment 37, wherein the mutation is a L to I substitution at position 53, 56, 80, or 118 of SEQ ID NO: 6.
39. The polypeptide of embodiment 1, 5, 18-23, or 36 or 38, wherein the IL-2 mutein comprises a IL-2 sequence of SEQ ID NO: 6, further comprising a mutation at one or more positions of 29, 31, 35, 37, 48, 69, 71, 74, 88, and 125 of SEQ ID NO: 6.
40. The polypeptide of embodiment 37, wherein the mutation is a L to I substitution at position 53, 56, 80, or 118 of SEQ ID NO: 60.
41. The polypeptide of embodiment 1, 5, 18-23, or 37, wherein the IL-2 mutein comprises a IL-2 sequence of SEQ ID NO: 60, further comprising a mutation at one or more positions of 29, 31, 35, 37, 48, 69, 71, 74, 88, and 125 of SEQ ID NO: 60.
42. The polypeptide of embodiment 1, 5, 18-23, or 36-41, wherein the IL-2 mutein further comprises a mutation at one or more of positions of E15, H16, Q22, D84, E95, or Q126.
43. The polypeptide of embodiment 1, 5, 18-23, or 36-42, wherein the mutation in the IL-2 mutein is a mutation at one or more of E15Q, H16N, Q22E, D84N, E95Q, or Q126E.
44. The polypeptide of embodiment 1, 5, 18-23, or 36-43, wherein the IL-2 mutein comprises a N29S mutation.
45. The polypeptide of embodiment 1, 5, 18-23, or 36-44, wherein the IL-2 mutein comprises a Y31S or a Y51H mutation.
46. The polypeptide of embodiment 1, 5, 18-23, or 36-45, wherein the IL-2 mutein comprises a K35R mutation.
47. The polypeptide of embodiment 1, 5, 18-23, or 36-46, wherein the IL-2 mutein comprises a T37A mutation.
48. The polypeptide of embodiment 1, 5, 18-23, or 36-47, wherein the IL-2 mutein comprises a K48E mutation.
49. The polypeptide of embodiment 1, 5, 18-23, or 36-48, wherein the IL-2 mutein comprises a V69A mutation.
50. The polypeptide of embodiment 1, 5, 18-23, or 36-49, wherein the IL-2 mutein comprises a N71R mutation.
51. The polypeptide of embodiment 1, 5, 18-23, or 36-50, wherein the IL-2 mutein comprises a Q74P mutation.
52. The polypeptide of embodiment 1, 5, 18-23, or 36-51, wherein the IL-2 mutein comprises a N88D or a N88R mutation.
53. The polypeptide of embodiment 1, 5, 18-23, or 36-52, wherein the IL-2 mutein comprises a C125A or C125S mutation.
54 The polypeptide of embodiment 1, 5, 18-23, or 36-53, wherein the IL-2 mutein is fused or linked to a Fc peptide, such as a protein comprising a sequence of SEQ ID NO: 59.
55. The polypeptide of embodiment 54, wherein the Fc peptide comprises a mutation at one or more of positions of L234, L247, L235, L248, G237, and G250.
56. The polypeptide of embodiment 54, wherein the mutation in the Fc polypeptide is L to A or G to A mutation.
57. The polypeptide of embodiment 54, wherein the Fc peptide comprises L247A, L248A, and G250A mutations.
58. The polypeptide of embodiment 54, wherein the Fc peptide comprises a L234A mutation, a L235A mutation, and/or a G237A mutation.
59. The polypeptide of embodiment 1, wherein the polypeptide comprises a first chain and a second chain that form the polypeptide, wherein
   the first chain comprises:
   V_{H}-H_{c}-Linker-C₁, wherein V_{H} is a variable heavy domain that binds to the target kidney cell with a V_{L} domain of the second chain; H_{c} is a heavy chain of antibody comprising CH1-CH2-CH3 domain, the Linker is a glycine/serine linker, and C₁ is an IL-2 mutein fused or linked to a Fc protein in either the N-terminal or C-terminal orientation; and
   the second chain comprises:
   V_{L}-L_{c}, wherein V_{L} is a variable light chain domain that binds to the target cell with the V_{H} domain of the first chain, and the Lc domain is a light chain CK domain.
60. The polypeptide of embodiment 59, wherein the VH and VL domain are an anti-CDH16 antibody or an anti-OCT2 antibody variable domains that bind to CDH16 or OCT2, respectively, expressed on a cell.
61. The polypeptide of embodiment 59, wherein the IL-2 mutein comprises a mutation at a position that corresponds to position 53, 56, 80, or 118 of SEQ ID NO: 6.
62. The polypeptide of embodiment 61, wherein the mutation is a L to I mutation at position 53, 56, 80, or 118 of SEQ ID NO: 6.
63. The polypeptide of embodiment 59, wherein the IL-2 mutein comprises a mutation at a position that corresponds to position 53, 56, 80, or 118 of SEQ ID NO: 60.
64. The polypeptide of embodiment 63, wherein the mutation is a L to I mutation at position 53, 56, 80, or 118 of SEQ ID NO: 60.
65. The polypeptide of any one of embodiments 59 or 61, wherein the mutein further comprises a mutation at a position that corresponds to position 69, 75, 88, or 125, or any combination thereof.
66. The polypeptide of embodiment 59, comprises a mutation selected from the group consisting of: at one of L53I, L56I, L80I, and L118I and the mutations of V69A, Q74P, N88D or N88R, and optionally C125A or C125S.
67. The polypeptide of any one of embodiments 59-66, wherein the IL-2 mutein comprises a L53I mutation.
68. The polypeptide of any one of embodiments 59-66, wherein the IL-2 mutein comprises a L56I mutation.
69 The polypeptide of any one of embodiments 59-66, wherein the IL-2 mutein comprises a L80I mutation.
70. The polypeptide of any one of embodiments 59-66, wherein the IL-2 mutein comprises a L118I mutation.
71. The polypeptide of any one of embodiments 59-66, wherein the IL-2 mutein does not comprises any other mutations.
72. The polypeptide of any one of embodiments 59-66, wherein the Fc protein comprises L247A, L248A, and G250A mutations, or a L234A mutation, a L235A mutation, and/or a G237A mutation according to Kabat numbering.
73. The polypeptide of any one of embodiments 59-66, wherein the Linker comprises a sequence of GGGGSGGGGGGGGS (SEQ ID NO: 30) or GGGGSGGGGSGGGGSGGGGS (SEQ ID NO: 22).
74. The polypeptide of any one of embodiments 1-73, wherein the kidney targeting moiety is an antibody that binds to CDH16 or OCT2.
75. The polypeptide of embodiment 74, wherein the antibody comprises a sequence as provided for herein.
76. The polypeptide of any one of embodiments 1-75, wherein the PD-1 agonist is an antibody selected from the PD-1 Antibody Table.
77. The polypeptide of any of the preceding embodiments, wherein the effector binding/modulating moiety selectively binds to a receptor expressed on the surface of a cell.
78 The polypeptide of embodiment 77, wherein the receptor is an IL-2R.
79 The polypeptide of embodiment 77, wherein the cell is an immune cell.
80 The polypeptide of embodiment 79, wherein the immune cell is a CD25 expressing cell.
81. The polypeptide of embodiment 80, wherein the CD25 expressing cell is a Treg.
82. The polypeptide of any of the preceding embodiments, wherein the polypeptide localizes to the kidney.
83. The polypeptide of embodiment 82, wherein the effector binding/modulating moiety selectively binds to a receptor expressed on the surface of a cell.
84 The polypeptide of embodiment 83, wherein the receptor is an IL-2R.
85. The polypeptide of embodiment 83, wherein the cell is an immune cell.
86 The polypeptide of embodiment 85, wherein the immune cell is a CD25 expressing cell.
87 The polypeptide of embodiment 86, wherein the CD25 expressing cell is a Treg.
88. The polypeptide of embodiment 83, wherein the effector binding/modulating moiety binding to the Treg causes Treg expansion.
89 The polypeptide of embodiment 88, wherein the Treg expansion is localized to the kidney, such as the kidney tubules.
90. A method of treating a subject with a kidney disorder comprising administering a therapeutically effective amount of a polypeptide of any of embodiments 1-89 to the subject to treat the disorder.
91. The method of embodiment 90, wherein the kidney disorder is Goodpasture's Syndrome (anti-GBM disease), inflammatory renal disease, glomerulonephritis, nephritis, lupus, lupus nephritis, IgA nephritis, membranous nephropathy, membranoproliferative glomerulonephritis, acute kidney injury, and chronic kidney disease, focal segmented glomerular sclerosis (FSGS), lupus nephritis, systemic scleroderma, membranous glomerular nephropathy (MGN), membranous nephropathy (MN), minimal change disease (MCD), IgA nephropathy, ANCA-associated vasculitis (AAV), Sjogren's syndrome, and Scleroderma, systemic sclerosis (SSc), or graft versus host disease (GVHD) of a kidney transplant.
92. A method of treating GVHD comprising administering a therapeutically effective amount of the polypeptide of any of embodiments 1-89 to the subject to treat the GVHD.
93. A method of treating GVHD of the kidney comprising administering a therapeutically effective amount of the polypeptide of any of embodiments 1-89 to the subject to treat the GVHD of the kidney.
94. A method of treating a subject who has had a transplant, such as a kidney transplant, comprising administering a therapeutically effective amount of the polypeptide of any of embodiments 1-89 to the subject, thereby treating the subject.
95. A method of treating GVHD in a subject having a transplanted donor tissue comprising administering a therapeutically effective amount of the polypeptide of any of embodiments 1-89 to the subject.
96. A method of treating a subject having, or at risk, or elevated risk, for having, an autoimmune disorder, comprising administering a therapeutically effective amount of the polypeptide of any embodiments 1-89, thereby treating the subject.
97. A nucleic acid encoding a polypeptide of any one of embodiments 1-89.
98. A vector comprising the nucleic acid of embodiment 97.
99. A cell comprising the nucleic acid of embodiment 97 or the vector of embodiment 98.
100. A method of making a polypeptide comprising culturing a cell of embodiment 99 to make the polypeptide.
101. A method of making a nucleic acid sequence encoding a polypeptide of embodiment 1-89, comprising
   a) providing a vector comprising sequence encoding a targeting moiety and inserting into the vector sequence encoding an effector binding/modulating moiety to form a sequence encoding the polypeptide; or
   b) providing a vector comprising sequence encoding an effector binding/modulating moiety and inserting into the vector sequence encoding a targeting moiety to form a sequence encoding the polypeptide,
   thereby making a sequence encoding the polypeptide.
102. A pharmaceutical composition comprising a polypeptide of any one of embodiments 1-89 and a pharmaceutically acceptable carrier.
103. A protein comprising a polypeptide that binds to CDH16 or OCT2 and a polypeptide that binds to PD-1.
104. The protein of embodiment 103, wherein the polypeptide that binds to CDH16 or OCT2 is an antibody.
105. The protein of embodiment 103, wherein the polypeptide that binds to PD-1 is an antibody.
106. The protein of embodiment 105, wherein the PD-1 antibody is a PD-1 agonist.
107. A pharmaceutical composition comprising the polypeptide of any one of embodiments 1-90, or the protein of any one of embodiments 103-106 and a pharmaceutically acceptable carrier.

The following examples are illustrative, but not limiting, of the compounds, compositions and methods described herein. Other suitable modifications and adaptations known to those skilled in the art are within the scope of the following embodiments.

### EXAMPLES

### Example 1: CD39 and/or CD73 as Effector Domains Creating a Purinergic Halo Surrounding a Cell Type or Tissue of Interest

A catalytically active fragment of CD39 and/or CD73 is fused to a targeting domain. Upon binding and accumulation at the target site, CD39 phosphohydrolyzes ATP to AMP. Upon binding and accumulation at the target site, CD73 dephosphorylates extracellular AMP to adenosine. A soluble catalytically active form of CD39 suitable for use herein has been found to circulate in human and murine blood, see, e.g., Yegutkin et al FASEB J. 2012 September; 26 (9): 3875-83. A soluble recombinant CD39 fragment is also described in Inhibition of platelet function by recombinant soluble ecto-ADPase/CD39, Gayle et al J Clin Invest. 1998 May 1; 101 (9): 1851-1859. A suitable CD73 molecule comprises a soluble form of CD73 which can be shed from the membrane of endothelial cells by proteolytic cleavage or hydrolysis of the GPI anchor by shear stress see, e.g., reference: Yegutkin G, Bodin P, Burnstock G. Effect of shear stress on the release of soluble ecto-enzymes ATPase and 5′-nucleotidase along with endogenous ATP from vascular endothelial cells. Br J Pharmacol 2000; 129:921-6.

The local catalysis of ATP to AMP or AMP to adenosine will deplete local energy stores required for fulminant T effector cell function. Treg function should not be impacted by ATP depletion due to their reliance on oxidative phosphorylation for energy needs (which requires less ATP), wherein T memory and other effector cells should be impacted due their reliance on glycolysis (requiring high ATP usage) for fulminant function.

### Example 2: A Bifunctional Polypeptide was Made in Different Orientations

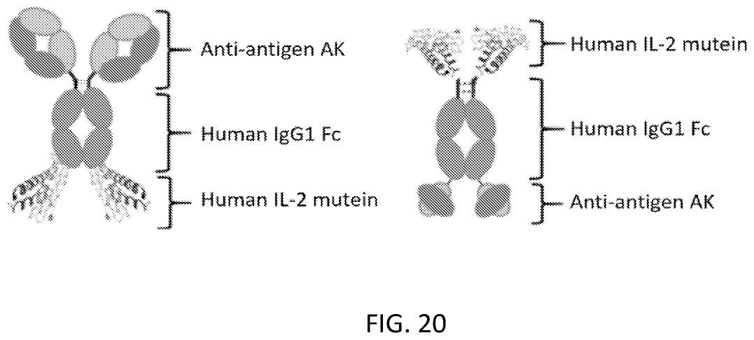

A bifunctional antibody with a IL-2 mutein at the C-terminus and anti-CDH16 antibody in IgG1 format and also a bifunctional antibody with IL-2M at the N-terminus and anti-CDH16 antibody in scFv format. These are represented in FIG. 20. Anti-antigen AK refers to the antibody that binds to CDH16. The anti-CDH16 antibody could also be replaced with anti-OCT2 antibody.

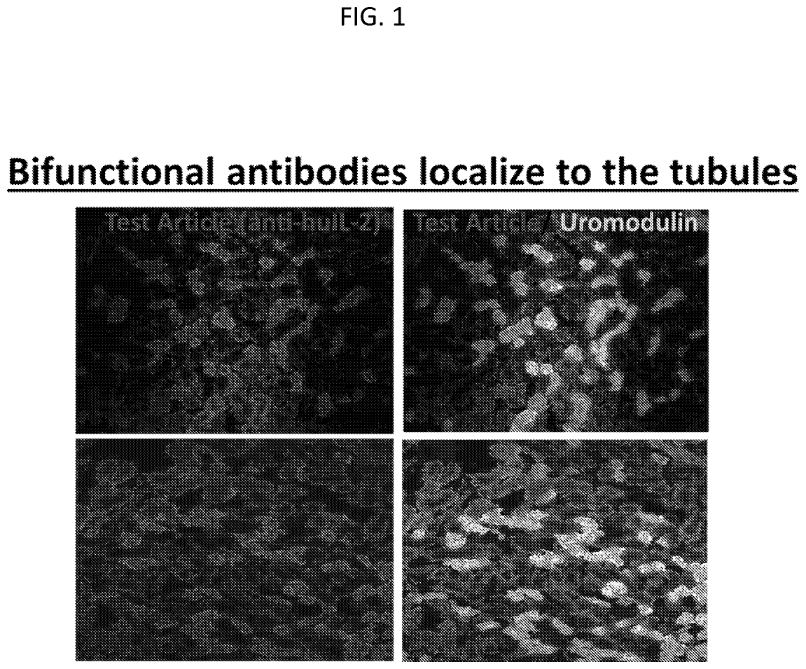

### Example 3: The Bi-Functional Polypeptides of Example 2 were Found to be Localized to the Tubules. (FIG. 1)

Bifunctional antibodies comprising IL-2 mutein effector moiety and an anti-CDH16 antibody targeting moiety bind to mouse kidney tissue ex vivo at 10 nM and co-localize with the tubule marker uromodulin. Fresh frozen mouse kidney tissue sections (5 um) were fixed with acetone, blocked, then stained with test articles (TA) and a uromodulin polyclonal antibody.

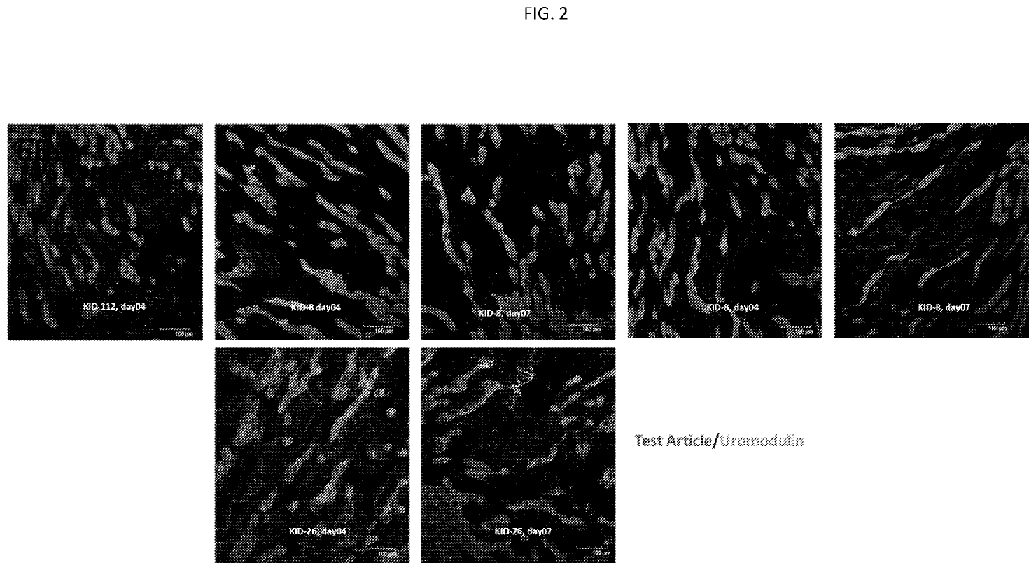

The molecules were then tested to determine if they can bind to human and cyno kidney ex vivo, which they were at 10 nM (FIG. 2). Briefly, fresh frozen human and cyno kidney tissue blocks were fixed with acetone, blocked, then stained with TAs, anti-uromodulin, as well as a commercial AK antibody as a control. Images were analyzed using confocal microscopy. The molecules were also found to localize to mouse kidney tubular epithelium in vivo. Mice were subcutaneously injected with either 3 mpk or 0.3 mpk of TAs. Kidney tissue was harvested at day 4 and day 7. Tissues were then sectioned at 5 um, fixed with acetone, blocked, and stained with uromodulin and anti-human IgG for TA detection. No localization was observed to other tissues tested.

These results demonstrate that bi-functional molecules comprising an anti-CDH16 antibody and immune effector domain, such as an IL-2 mutein, or PD-1 agonist, or CD39 effector domain could be used to modulate the immune system specifically at the kidney. These molecules can then be used to treat various conditions where an active immune system is detrimental to the kidney, such as a kidney transplant. Thus, patients with end renal disease that require a kidney transplant can be treated with such bi-functional molecules.

### Example 4: Kidney-Targeting Bispecifics Localize to the Kidney and Selectively Bind Tregs

Regulatory T cells (Treg) play a critical role in maintaining graft tolerance following organ transplantation, and increased numbers of Tregs in solid-tissue grafts such as the kidney are associated with improved graft survival and function. Tregs are also able to suppress the activity of many other proinflammatory cell types, including the immune cells that infiltrate the kidney tubules in autoimmune disease like lupus nephritis. Therapeutic approaches that increase Tregs offer a promising alternative to current standard-of-care treatment with broad-acting immunosuppressants and their attendant side effects.

Without wishing to be bound by a particular theory, low dose (LD) interleukin 2 (IL-2) drives Treg proliferation and function via the heterotrimeric IL-2 receptor expressed on Tregs and has been shown to expand Tregs in vivo, including in the context of transplantation and acute inflammation. However, IL-2 can also activate other immune cells including conventional T cells and Natural Killer (NK) cells, which express IL-2Rβ/CD122 and IL-2β/CD132, and which could accelerate rejection. IL-2 selectivity for Tregs was enhanced by introducing mutations, as provided herein, that increase affinity for CD25 and decrease affinity for CD122/CD132, resulting in Treg specific expansion.

Wild type mice were separately given a single subcutaneous dose of two kidney-tethered (comprising an anti-CDH16 antibody targeting moiety) IL-2M bispecific molecules. Kidneys were collected after 4 days, frozen, sectioned, and stained with anti-huIgG to detect kidney-tethered IL-2M bispecific molecules. Tissue sections were also stained with uromodulin as a tubule marker. Additionally, cyno kidney tissue was stained separately ex vivo with two kidney-tethered IL-2M bispecific molecules, as provided herein, followed by detection of the test article with anti-huIL2. A commercial antibody for the tubule target protein was included as a counterstain. Both kidney-tethered IL-2M bispecific molecules were detected in mouse kidney tubules after in vivo dosing, and both kidney-tethered IL-2M bispecific molecules stained cyno kidney tissue.

These data show that kidney targeting bispecific molecules comprising IL-2M and a tubule tether localize to the kidney and specifically to the kidney tubules.

### Example 5: Kidney-Targeting Bispecifics Retain Treg pSTAT5 Selectivity

Plates were either coated overnight with anti-huIgG or were seeded with engineered cell lines expressing the kidney tether protein. Kidney-tethered (comprising an anti-CDH16 antibody targeting moiety) IL-2M bispecific molecules were added and incubated for 1 hr. Unbound kidney-tethered IL-2M bispecific molecules were removed by washing and freshly isolated PBMCs from healthy human donors were added and incubated for 1 hr. Cells were harvested, and the amount of STAT5 phosphorylation was determined by flow cytometry and shown here as percent of total population positive for pSTAT5. The data showed increase in percent of total Treg population positive for pSTAT5 after treatment with the kidney-tethered IL-2M bispecific molecules, suggesting activation of Tregs by the kidney-tethered IL-2M bispecific molecules. Other cells did not exhibit STAT5 phosphorylation.

Accordingly, IgG-captured and cell bound kidney-tethered IL-2M bispecific molecules retain Treg pSTAT5 selectivity.

### Example 6: Kidney-Targeting Bispecifics Demonstrate Prolonged and Tissue-Specific PK

NSG mice were dosed subcutaneously with kidney-tethered (comprising an anti-CDH16 antibody targeting moiety) IL-2M bispecific molecules at either 1 or 10 mpk and tissues were collected at days 4, 7, 10 and 14. An MSD-based generic ELISA was developed and used to determine tissue concentration of the kidney-tethered IL-2M bispecific molecule. Plates were coated with anti-human IgG and tissue lysates were added, then unbound samples were washed away and captured kidney-tethered IL-2M bispecific molecules were detected by anti-huKappa. Data was subsequently normalized to pg/mg tissue. The data demonstrated prolonged and tissue-specific PK up to 14 days. The bispecific molecule comprising an IL-2M and an anti-tubule-specific protein tether, as provided in any of the above embodiments, demonstrates prolonged and site-specific PK.

### Example 7: Kidney-Targeting Bispecifics Localize to the Kidney and Lead to Treg Expansion

The bispecific molecule comprising and IL-2M and an anti-CDH16 antibody kidney tether, as provided herein, was used to subcutaneously dose CD34-engrafted NSG mice. At days 7 and 12 kidneys and peripheral blood were analyzed by flow cytometry for Treg expansion. On day 7 both systemic IL-2M and kidney-tubule-tethered IL-2M induced peripheral Treg expansion in the blood, but by day 12 Treg numbers largely returned to baseline. In contrast, the kidney-tethered IL2M bispecific molecule induced greater kidney Treg expansion on day 7 and said expansion was maintained for at least 12 days after dosing, compared to the systemic IL-2M. The animals dosed with the kidney-tethered IL-2M, as provided in any of the embodiments above, exhibited prolonged kidney Treg expansion compared to a systemic, non-tethered IL-2M.

The Examples provided herein demonstrate that molecules provided herein can be used to specifically localize therapeutics at the kidney, such as an IL-2 mutein, PD-1 agonist, or CD39 Effector Domains, and also other therapeutic molecules, such as those described herein. The Example provided herein also demonstrate the potential of the kidney-targeting IL-2M bispecific molecules to substantially improve kidney graft acceptance following transplant, or decrease inflammation in the kidney that is associated with autoimmune disease, while reducing adverse effects and improving patient quality of life.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1. A comprising a kidney targeting moiety, wherein the kidney targeting moiety comprises an anti-CDH16 antibody or an anti-OCT 2 antibody and an effector binding/modulating moiety, wherein the effector binding/modulating moiety is a PD-1 agonist, a CD39 Effector Domain, or an IL-2 mutein polypeptide (IL-2 mutein).

2-35. (canceled)

36. The protein of claim 1, wherein the effector binding/modulating moiety is an IL-2 mutein comprising an IL-2 sequence of SEQ ID NO: 6 which comprises a mutation at a position that corresponds to position 53, 56, 80, or 118 of SEQ ID NO: 6 and optionally one or more additional mutations of SEQ ID NO:6.

37. The protein of claim 1, wherein the effector binding/modulating moiety is an IL-2 mutein comprising the sequence of SEQ ID NO: 60, wherein at least one of X₁, X₂, X₃, and X₄ is I and the remainder are L or I, wherein the IL-2 mutein optionally comprises one or more additional mutations of SEQ ID NO:60.

38. The protein of claim 36, wherein the mutation is a L to I substitution at position 53, 56, 80, or 118 of SEQ ID NO: 6.

39. The protein of claim 36, wherein the IL-2 mutein, further comprises a mutation at one or more positions of 29, 31, 35, 37, 48, 69, 71, 74, 88, and 125 of SEQ ID NO: 6.

40. The protein of claim 37, wherein the IL-2 mutein comprises an L to I substitution at position 53, 56, 80, or 118 of SEQ ID NO: 60.

41. The protein of claim 37, wherein the IL-2 mutein further comprises a mutation at one or more positions of 29, 31, 35, 37, 48, 69, 71, 74, 88, and 125 of SEQ ID NO: 60.

42. The protein of claim 41, wherein the IL-2 mutein further comprises a mutation at one or more of positions of E15, H16, Q22, D84, E95, or Q126.

43. The protein of claim 42, wherein the IL-2 mutein comprises an E15Q, H16N, Q22E, D84N, E95Q, or Q126E substitution.

44. The protein of claim 43, wherein the IL-2 mutein comprises a N29S, Y31S, Y31H, K35R, T37A, K48E, V69A, N71R, Q74P, N88D, N88R, C125A or C125S mutation.

45-53. (canceled)

54. The protein of claim 1, wherein the effector binding/modulating moiety is an IL-2 mutein that is fused or linked to a Fc peptide, wherein the Fc peptide comprises a sequence of SEQ ID NO: 59, optionally comprising one or more mutations of SEQ ID NO:59.

55. The protein of claim 54, wherein the Fc peptide comprises a mutation at one or more of positions of L234, L247, L235, L248, G237, and G250 of SEQ ID NO: 59.

56. The protein of claim 54, wherein the mutation in the Fc polypeptide is an L to A or G to A mutation.

57. The protein of claim 54, wherein the Fc peptide comprises L247A, L248A, and G250A mutations.

58. The protein of claim 54, wherein the Fc peptide comprises a L234A mutation, a L235A mutation, and/or a G237A mutation.

59. The protein of claim 1, wherein the protein comprises a first chain and a second chain, wherein
the first chain comprises:
V_{H}-H_{c}-Linker-C₁, wherein the V_{H} is a variable heavy domain that binds to the target kidney cell with a V_{L} domain of the second chain; the H_{c} is a heavy chain comprising a CH1-CH2-CH3 domain, the Linker is a glycine/serine linker, and C₁ is an IL-2 mutein fused or linked to a Fc protein in either the N-terminal or C-terminal orientation; and
the second chain comprises:
V_{L}-L_{c}, wherein the V_{L} is a variable light chain domain that binds to the target cell with the V_{H} domain of the first chain, and the Lc domain is a light chain CK domain.

60. The protein of claim 59, wherein the V_{H} and V_{L} domain are an anti-CDH16 antibody or an anti-OCT2 antibody variable domains that bind to CDH16 or OCT2, respectively, expressed on a cell.

61. The protein of claim 59, wherein the IL-2 mutein comprises a mutation at a position that corresponds to position 53, 56, 80, or 118 of SEQ ID NO: 6.

62. The protein of claim 61, wherein the mutation is a L to I mutation at position 53, 56, 80, or 118 of SEQ ID NO: 6.

63. The protein of claim 59, wherein the IL-2 mutein comprises a mutation at a position that corresponds to position 53, 56, 80, or 118 of SEQ ID NO: 60.

64. The protein of claim 63, wherein the mutation is a L to I mutation at position 53, 56, 80, or 118 of SEQ ID NO: 60.

65. The protein of claim 61, wherein the mutein further comprises a mutation at a position that corresponds to position 69, 75, 88, or 125, or any combination thereof.

66. The protein of claim 59, wherein the IL-2 mutein comprises a mutation selected from the group consisting of: L53I, L56I, L80I, and L118I and the mutations of V69A, Q74P, N88D or N88R, and optionally C125A or C125S.

67. The protein of claim 66, wherein the IL-2 mutein comprises a L53I mutation.

68. The protein of claim 66, wherein the IL-2 mutein comprises a L56I mutation.

69. The protein of claim 66, wherein the IL-2 mutein comprises a L80I mutation.

70. The protein of claim 66, wherein the IL-2 mutein comprises a L118I mutation.

71. The protein of claim 66, wherein the IL-2 mutein does not comprises any other mutations.

72. The protein of claim 66, wherein the Fc protein comprises L247A, L248A, and G250A mutations according to Kabat numbering, or a L234A mutation, a L235A mutation, and/or a G237A mutation according to EU numbering.

73. The protein of claim 59, wherein the Linker comprises a sequence of GGGGSGGGGSGGGGS (SEQ ID NO: 30) or GGGGSGGGGSGGGGSGGGGS (SEQ ID NO: 22).

74-107. (canceled)
